(19) United States
(12) Patent Application Publication
(10) Pub. No.: US 2025/0125046 A1
(43) Pub. Date: Apr. 17, 2025

(54) SYSTEM AND METHOD FOR SECURE HEALTHCARE PROFESSIONAL COMMUNICATION

(71) Applicant: Optiks Solutions, Inc., Edison, NJ (US)

(72) Inventors: Anupam Nandwana, Edison, NJ (US); Edward VAZ, Edison, NJ (US)

(21) Appl. No.: 18/957,691

(22) Filed: Nov. 23, 2024

Related U.S. Application Data
(63) Continuation-in-part of application No. 17/751,931, filed on May 24, 2022.
(60) Provisional application No. 63/192,572, filed on May 25, 2021.

(51) Int. Cl. G16H 40/67

(52) U.S. Cl. CPC G16H 40/67

(57) ABSTRACT

A system and method for secure healthcare professional communication with patients that employs a variety of different messaging modalities. Examples of suitable messaging modalities that may be used with the present invention include but are not limited to SMS, Facebook Messenger, WhatsApp, WeChat and other such modalities. Optionally and preferably, such modalities may be employed with the present invention but without requiring additional plug-ins, software and/or other apps to the communication device of the healthcare professional and/or the patient.

### FIELD OF THE INVENTION

The present invention, in at least some embodiments, is of a system and method for secure healthcare professional communication, and in particular, to such a system and method securely employing a variety of different messaging modalities with a variety of different roles, including without limitation healthcare professionals, office staff, external support staff or patients or caregivers thereof.

### BACKGROUND OF THE INVENTION

Communication with healthcare professionals is very important. Such individuals communicate with others in a variety of different roles, such as other healthcare professionals, office staff, external support staff or patients or caregivers thereof. Security and confidentiality are both important to maintain patient privacy, even when communication is not occurring directly with patients. However convenience is also important; individuals today have become accustomed to the convenience of direct messaging and/or voice calls through smart phones and other personal communication devices. Such convenience may permit more rapid communication which is also important in today's high pressure world of healthcare. Combining both convenience and security can be quite difficult.

### BRIEF SUMMARY OF THE INVENTION

The present invention in at least some embodiments, is of a system and method for secure healthcare professional communication with individuals in a variety of different roles that employs a variety of different messaging modalities, while maintaining security and convenience across all of the different roles and modalities. Examples of individuals with different roles with whom such communication may be performed include but are not limited to healthcare professionals, office staff, external support staff, or patients or caregivers thereof. External support staff may include staff at diagnostic laboratories, other medical centers, and staff at providers of goods and services to healthcare professionals, including without limitation sales staff, pharmacists, medical device specialists, scientists and the like.

Optionally and preferably, the messaging modality comprises a modality selected from the group consisting of a modality addressed through a telephone number, a social media modality, an email modality, a voice modality, a chat modality and a video modality. More preferably, the messaging modality comprises a general messaging modality. By “general messaging modality” it is meant a messaging modality that is installed for general use, although it may be directed toward a particular messaging modality. However, such a messaging modality is more preferably not purpose-built only for healthcare professionals for example. Nonetheless, the present invention does encompass such purpose-built messaging modalities.

Examples of suitable messaging modalities that may be used with the present invention include but are not limited to SMS (Short Message Service), MMS (Multimedia Messaging Service), RCS (Rich Communication Services), Facebook Messenger, WhatsApp, WeChat, iMessage, Apple Business Chat, Telegram, Signal, Skype message and other such modalities. Optionally and preferably, such modalities are suitable for being employed with the present invention but without requiring additional plug-ins, software and/or other apps to the communication device of the healthcare professional and/or the patient. The term “patient” as used herein may refer to a patient receiving care and/or a caregiver.

According to at least some embodiments, there is provided a system for supporting secure healthcare professional communication, comprising a computer network, a sending user computational device, a server and a recipient user computational device, wherein said sending user computational device, said server and said recipient user computational device communicate through said computer network; wherein each of said sending user computational device, said recipient user computational device and said server comprises a memory for storing instructions for supporting functions of each, and a processor for executing said instructions; wherein each of said sending user computational device and said recipient user computational device comprises a messaging modality, wherein said messaging modality comprises a selected from the group consisting of a modality addressed through a telephone number, a social media modality, an email modality, a voice modality, a chat modality, a text modality and a video modality; wherein said sending user computational device creates a message according to a template determined according to at least one policy; wherein said message is sent from said sending user computational device to said server and is addressed according to an address associated with said messaging modality; and wherein said server relays said message according to said address to said recipient user computational device; wherein said address of said sending user computational device and said recipient user computational device are each masked.

Optionally said address is selected from the group consisting of a telephone number, a social media address, an email address, a voice modality address, a chat address and a video transmission address. Optionally said video transmission address comprises one or more of said social media address, said email address, said chat address or a live streaming video interface. Optionally said messaging modality is transmitted through a separate network to said recipient user computational device, wherein said server sends said message to said separate network. Optionally said separate network is selected from the group consisting of a telephone network, a cellular network, an internet, and a messaging modality company server connected through any of said telephone network, said cellular network and said internet. Optionally said sending user computational device comprises an external support staff user computational device and said recipient user computational device comprises a healthcare professional computational device, wherein the device is controllable by a healthcare professional. Optionally said external support staff comprises staff at diagnostic laboratories, other medical centers, and staff at providers of goods and services to healthcare professionals, including without limitation sales staff, pharmacists, medical device specialists and scientists; wherein said sending user computational device is associated with said external support staff and wherein said policy is enforced according to an external support staff organization. Optionally said telephone number is associated with a mobile device connected to or identified with an external support staff member or an external support staff organization. Optionally said telephone number is associated with a mobile device connected to or identified with a healthcare professional or a healthcare professional office. Optionally said message comprises an SMS (Short Message Service) message, an MMS (Multimedia Messaging Service) message or a RCS (Rich Communication Services). Optionally said social media address comprises one or more of Facebook Messenger, WhatsApp, WeChat, iMessage, Apple Business Chat, Telegram, Signal, or Skype; and wherein said message is sent and/or received through said social media channel. Optionally said messaging modality comprises a general purpose messaging modality. Optionally said general purpose messaging modality is selected from the group consisting of SMS (Short Message Service), MMS (Multimedia Messaging Service), RCS (Rich Communication Services), email, email chat functions, internet based chat, internet based voice communication, internet based video communication and other such modalities. Optionally said general purpose messaging modality comprises one or more of Facebook Messenger, WhatsApp, WeChat, iMessage, Apple Business Chat, Telegram, Signal, and Skype. Optionally said messaging modality does not require additional plug-ins and/or software on said sending user computational device and said recipient user computational device, beyond what is required for said general purpose messaging modality.

Optionally, wherein said server further comprises a content analysis function, wherein a content of said message is analyzed for compliance with a content policy. Optionally said content policy is determined according to a healthcare regulation or law. Optionally said content policy is determined according to an organization controlling said sending user computational device, wherein said organization is selected from the group consisting of a healthcare organization and a sales organization. Optionally said content analysis function comprises an AI engine for analyzing said content for content moderation. Optionally said AI engine analyses said content according to a natural language processing (NLP) machine learning algorithm. Optionally said AI engine analyses said content according to an image processing machine learning algorithm. Optionally said AI engine comprises a machine learning algorithm selected from the group consisting of a CNN (convolutional neural network), a DBN (deep belief network), a BiLS™ (bilateral long short term memory network), Sentence-Transformers, BERT (Bidirectional Encoder Representations from Transformers), modifications of BERT, another type of RNN, logistic regression models for NLP, Naïve Bayesian models, Generative Pre-Trained Transformer (GPT), and Mixture of Experts (MoE). Optionally said AI engine further receives additional information about a sending user and a recipient user, and further analyzes said content according to said additional information. Optionally if said AI engine determines that said message from said sending user computational device is determined to be acceptable under said content policy, said AI engine permits said message to be transmitted to said recipient user computational device.

According to at least some embodiments, the system further comprises a supervisory user computational device, wherein if said message from said sending user computational device is determined to violate said content policy by said AI engine, said AI engine sends said message to said supervisory user computational device for review. Optionally said AI engine further logs said content and a result of a review of said content according to said content policy, with said additional information if available, at a data storage. Optionally said content policy is determined according to an organization controlling said sending user computational device, wherein said organization is a healthcare organization and said sending user is a healthcare professional. Optionally said recipient user is a patient, and wherein said content policy comprises at least one rule related to communication between said healthcare professional and said patient. Optionally said content policy is determined according to an organization controlling said sending user computational device, wherein said organization is a sales organization. Optionally said recipient user is a healthcare professional, and wherein said content policy comprises at least one rule related to communication between said healthcare professional and said sales organization. Optionally said AI engine further comprises a coaching function; wherein said content of said message is analyzed for one or more of policy violations, clarity, word choice or tone; wherein said AI engine further determines that coaching is required according to said analysis of said content; wherein said coaching function of said AI engine provides one or more suggestions to adjust said content to said sending user computational device. Optionally said AI engine comprises a machine learning algorithm, and wherein said machine learning algorithm is trained on labelled data, wherein said labelled data comprises at least message content; wherein training said machine learning algorithm comprises analyzing said labelled data by said machine learning algorithm; calculating an error by said machine learning algorithm; adjusting said machine learning algorithm until said error is below a threshold. Optionally said error is calculated directly and said adjusting said machine learning algorithm comprises back propagation. Optionally said error is calculated indirectly, and said adjusting said machine learning algorithm comprises analyzing labelled data related to a desired outcome of said machine learning algorithm and also labelled data related to a non-desired outcome of said machine learning algorithm.

Optionally said server comprises a logging function for logging a content of messages sent by said sending user computational device to form logged content. Optionally said logged content is analyzed according to said policy. Optionally said message comprises live audio, live video or live chat, and wherein said server causes a delay in transmission of said message to apply a content moderation function. Optionally said text modality comprises a survey, wherein said survey is transmitted according to another messaging modality of any of the above claims. Optionally said recipient user computational device comprises a plurality of recipient user computational devices for a plurality of recipient users. Optionally said plurality of recipient computational devices communicate according to a messaging modality for supporting a group chat. Optionally said server applies a content moderation function according to whether said plurality of recipient users belong to a single organization. Optionally an organization of said sending user computational device is a sales organization and wherein an organization of said recipient user computational device is a healthcare organization, wherein said message relates to a request for a physical sample of a healthcare product, and wherein upon receipt of said physical sample, said recipient user computational device sends a return message acknowledging receipt.

According to at least some embodiments, there is provided a method for increasing reliability of messaging communication through a messaging modality, implemented according to the system as described herein. Optionally the method is adapted for implementation with a HCP (healthcare provider), wherein the messaging modality is executed through a computational device by the HCP, the method comprising: receiving a request for medical information through the messaging modality; generating, under control of one or more computer systems, a message that provides the medical information and that includes message content, and recipient identification data for the HCP; reviewing the message content for compliance by a computer system; sending the message to the HCP if compliance has been established; confirming a delivery of the message to the HCP; and generating an audit trail that is configured to create a historical record corresponding to said message. Optionally said reviewing the message content for compliance comprises analyzing the message content by a NLP algorithm and preventing the message from being sent if non-compliant content is detected. Optionally said non-compliant content comprises one or more stop words. Optionally said non-compliant content comprises content that is not appropriate for the HCP, according to an analysis of a license or other certification held by the HCP.

According to at least some embodiments, the method further comprises confirming a delivery of the message to the HCP; or alternatively, upon receiving indication of delivery failure of the message, sending the message via a different redundant communication modality. According to at least some embodiments, the method further comprises validating the message and/or a response from the HCP according to a contact detail and/or a parameter of the HCP. Optionally said parameter comprises one or both of a license of the HCP or an office address corresponding to a license of the HCP. Optionally the audit trail includes message timestamp data for the message, delivery attempt data including corresponding timestamp data, response data, identification data for the HCP and/or user identification data. Optionally transmitting the message through the messaging modality and the review for non-compliant content are compliant with healthcare and/or pharmaceutical service specific requirements when operated in combination.

According to at least some embodiments, there is provided a method for increasing reliability of messaging communication through a messaging modality with a patient, wherein the messaging modality is executed through a computational device by the patient, the method comprising: initiating transmission of patient-specific medical information through the messaging modality to the computational device by one or more of an HCP, HCP staff member and/or external support staff member; generating, under control of one or more computer systems, a message that provides the patient specific medical information and that includes message content, and recipient identification data for the patient; reviewing the message content for compliance by a computer system; sending the message to the patient if compliance has been established; confirming a delivery of the message to the patient; and generating an audit trail that is configured to create a historical record corresponding to said message. Optionally, said reviewing the message content for compliance comprises analyzing the message content by a NLP algorithm and preventing the message from being sent if non-compliant content is detected. Optionally, said non-compliant content comprises one or more stop words. Optionally, said non-compliant content comprises content that is not medically relevant to the patient. Optionally, said non-compliant content comprises content that includes patient specific medical details that are protected under medical regulations. Optionally, affirmative consent for such content to be transmitted through the messaging modality has not been provided by the patient.

Optionally, the method further comprises confirming delivery of the message to the patient; or alternatively, upon receiving indication of delivery failure of the message, sending the message via a different redundant communication modality. Optionally, the method further comprises validating the message and/or a response from the patient according to a contact detail of the patient. Optionally, the audit trail includes message timestamp data for the message, delivery attempt data including corresponding timestamp data, response data, identification data for the patient and/or user identification data for the transmitting user. Optionally, transmitting the message through the messaging modality and the review for non-compliant content are compliant with healthcare and/or pharmaceutical service specific requirements when operated in combination.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Unless otherwise defined, all technical and scientific terms used herein have the same meaning as commonly understood by one of ordinary skill in the art to which this invention belongs. The materials, methods, and examples provided herein are illustrative only and not intended to be limiting.

An algorithm as described herein may refer to any series of functions, steps, one or more methods or one or more processes, for example for performing data analysis.

Implementation of the apparatuses, devices, methods and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions. The processor is configured to execute a predefined set of operations in response to receiving a corresponding instruction selected from a predefined native instruction set of codes.

Software (e.g., an application, computer instructions) which is configured to perform (or cause to be performed) certain functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions-which can be a set of instructions, an application, software-which are operable on a computational device (e.g., a processor) to cause the computational device to conduct and/or achieve one or more specific functionality. Some embodiments are described with regard to a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 shows a non-limiting, exemplary system for healthcare communication;

FIG. 2 relates to a non-limiting, exemplary method for contact management;

FIG. 3 shows a non-limiting, exemplary method for initiating communication with a healthcare professional;

FIG. 4 shows a non-limiting, exemplary method for initiating communication with a staff member of an office of a healthcare professional;

FIG. 5 shows a non-limiting, exemplary method for initiating communication with a contact that was not previously known;

FIG. 6 shows a non-limiting, exemplary method for communication with a healthcare professional;

FIG. 7 shows a non-limiting, exemplary method for communication with a staff member of an office of a healthcare professional;

FIG. 8 shows a non-limiting, exemplary method for communication with a previously unknown contact;

FIG. 9 shows a non-limiting, exemplary method for sending a message with an attachment to a HCP contact and/or a HCP staff contact;

FIG. 10 shows a non-limiting, exemplary method for an opt-in to initiate communication through the system;

FIG. 11 shows a non-limiting, exemplary method for an opt-out to further communication through the system;

FIG. 12 shows a non-limiting, exemplary method for outbound voice communication from a healthcare professional to a patient;

FIG. 13 shows a non-limiting, exemplary method for inbound voice communication from a patient to a healthcare professional;

FIG. 14 relates to a non-limiting, exemplary method for calling a recipient who is not already present in the CRM;

FIG. 15 shows a non-limiting, exemplary system for signature capture;

FIG. 16 relates to a non-limiting, exemplary method for sending a MIR template to a contact;

FIG. 17 relates to a non-limiting, exemplary method for scanning a code, such as a QR code for initiating communication by a recipient;

FIG. 18 shows an exemplary, non-limiting process for transmitting the previously described code, such as a QR code for example, to the recipient;

FIG. 19 shows an exemplary, non-limiting process for handling an out of office message;

FIG. 20 shows a non-limiting, exemplary method for communication between an external support staff member and a recipient, who could be a healthcare professional (HCP) or HCP staff for example, using a specific example of a messaging service;

FIGS. 21A and 21B show a non-limiting, exemplary architecture for content moderation;

FIG. 22 relates to a non-limiting, exemplary method for content moderation of transmitted messages through the system as described herein;

FIG. 23 relates to a non-limiting, exemplary architecture for bot communication;

FIG. 24 relates to a non-limiting, exemplary method for bot communication;

FIG. 25 shows a non-limiting, exemplary method for communication logging into a database;

FIG. 26 relates to a non-limiting, exemplary architecture for a video call;

FIG. 27 relates to a non-limiting, exemplary method for a video call;

FIG. 28 relates to a non-limiting, exemplary architecture for scheduling a call;

FIG. 29 relates to a non-limiting, exemplary flow for scheduling a call;

FIG. 30 relates to a non-limiting, exemplary architecture for short URL creation and redirection;

FIG. 31 relates to a non-limiting, exemplary flow for short URL creation and redirection;

FIG. 32 relates to a non-limiting, exemplary architecture for a group chat;

FIG. 33 relates to a non-limiting, exemplary flow for a group chat;

FIG. 34 relates to a non-limiting, exemplary architecture for short code messaging;

FIG. 35 relates to a non-limiting, exemplary flow for short code messaging;

FIG. 36 relates to a non-limiting, exemplary method for handling a MIR request;

FIG. 37 relates to a non-limiting, exemplary method for handling a sample request;

FIG. 38 shows a system for supporting communication with regard to multiple modalities, while FIG. 39 shows a non-limiting, exemplary method for communication through such a system;

FIGS. 40 and 41 show the system of FIG. 38 in more detail;

FIGS. 42A and 42B show exemplary systems for content moderation;

FIGS. 43A and 43B relate to exemplary systems for AI engines for content moderation;

FIG. 44 relates to a non-limiting exemplary method for moderation of visual content;

FIG. 45 relates to a non-limiting exemplary method for training an AI model for moderation of visual content;

FIG. 46 relates to a non-limiting exemplary method for sending a survey;

FIG. 47 shows an exemplary system for coaching;

FIGS. 48A and 48B relate to exemplary systems for AI engines for coaching;

FIG. 49 relates to a non-limiting exemplary method for coaching;

FIG. 50 relates to a non-limiting exemplary method for training an AI model for coaching;

FIG. 51 relates to a non-limiting exemplary method for a group chat;

FIG. 52 relates to a non-limiting exemplary method for a video chat;

FIG. 53 relates to a non-limiting exemplary method for receiving payment;

FIG. 54 relates to a non-limiting exemplary method for sample requests;

FIG. 55 illustrates a flowchart for an event and expense flow method, including steps for user authentication, event creation, attendee management, and expense tracking;

FIG. 56A depicts a block diagram of an EdenHelp Generative AI system for question and answer processing, showing components for user interface, natural language understanding, and response generation;

FIG. 56B presents a block diagram of an EdenHelp Generative AI backend system, highlighting the flow of information from user input through various processing layers to response delivery;

FIG. 57 shows a flowchart for a shared number flow method, detailing the process of handling messages sent to a shared number and assigning them based on contact status and various rules;

FIG. 58 outlines an admin user authentication and access process, demonstrating the steps for an admin to log in as a specific user and perform actions on their behalf;

FIG. 59 illustrates a method for managing user coverage requests, depicting the processes for both the requesting user and the covering user in a system;

FIGS. 60A, 60B, and 60C present flowcharts for enhanced contact management methods, specifically addressing the creation of new staff contacts and updating mobile numbers for healthcare professionals (HCPs) and staff; and

FIG. 61 shows a non-limiting, exemplary system that features further routing and message content control.

### DESCRIPTION OF AT LEAST SOME EMBODIMENTS

Digital communications in regulated industries require additional management and control than such communication in non-regulated industries. For example, there are strict rules regarding communication between providers of medical services, termed herein “healthcare providers” (HCP), and/or their staff, and companies that support the HCP with products and/or services, such as pharmaceutical companies for example. HCPs wish to receive information about these products and/or services on demand, or at least with a minimum of delay. Companies wish to market their products and/or services to HCP. However, these communications are regulated by the relevant healthcare regulation body in that country, such as the FDA (Food and Drug Administration) in the US. These rules specifically govern the distinct roles and interactions between commercial personnel and medical personnel within such companies.

In a regulated area such as healthcare, a company may need to provide medically appropriate information, but also wishes to sell a product or service. Regulations in the US require that the provision of medically appropriate information be separated from the provision of commercial information. Medical personnel engage in non-promotional medical communications and respond to unsolicited medical requests, while commercial personnel and sales staff focus on commercial aspects. Regulations require that the provision of medically appropriate information be separated from the provision of commercial information. For example, medical personnel at a pharmaceutical company may provide scientific information about a drug, including therapeutic effects, side effects and other medical information; while sales staff may provide commercial information about the drug. This separation helps ensure medical information is provided independently from commercial influence.

As a non-limiting example, in order to maintain compliance, the following restrictions may be implemented: commercial personnel may not be permitted to request off-label product information from medical personnel, and/or to seek assistance from medical personnel to overcome prescribing barriers. Commercial personnel may not be permitted to request information about ongoing clinical trials, and/or to influence medical content development. On the other hand, medical personnel may not be permitted to participate in commercial strategy meetings or pricing discussions. While medical personnel may be permitted to share aggregated customer insights, they may not be allowed to share specific HCP prescribing information (that, what a particular doctor has prescribed in the past, for example).

In a digitally connected world, technology is required to separate these types of communications and to support provision of information through different communication channels, while maintaining these role distinctions. Joint external meetings between commercial and medical personnel require pre-approval and must have a legitimate business purpose, with content and roles clearly defined in advance.

HCPs may also wish to communicate with a company through channels that previously were not used for medical business communication, such as messaging apps such as WhatsApp, SMS messages and other messaging modalities. Such channels may support a faster response time, and hence more efficient provision of information, but also need careful management to avoid transgressing a regulation and/or providing incorrect information. The system is preferably able to ensure that communications are appropriately routed to either medical or commercial personnel based on the nature of the inquiry.

Consistency of messaging across multiple channels is also important. In addition, communications and interactions across multiple channels need to be logged for compliance purposes. Therefore, communication is both faster when distributed across multiple touchpoints and with multiple parties participating (such as different groups of personnel at a drug company, as appropriate for medical vs commercial communications), yet also needs to be managed in a centralized manner, for compliance logging, and to ensure information is accurate and consistent.

Different communication modalities also support faster provision of customized information. Given a greater understanding of, and research into, multi-drug interactions, effects of aging on dosage levels, treatment of comorbidities and other specialist medical information, HCPs now require information that is customized to a particular patient's circumstances. As patient compliance is also understood to be important, customized communication with or on behalf of a patient is also important. The methods and systems as described herein may be applied to communication with a patient, whether from an external support staff member, and/or an HCP or an HCP staff member.

Furthermore, according to at least some embodiments, the system as described herein comprises a content moderation function, which may be located at a server through which a message or other content passes before being sent to a recipient. The content analysis function preferably analyzes such content for compliance with a content policy that includes maintaining appropriate separation between medical and commercial communications. The content policy may be determined according to a healthcare regulation or law and organizational guidelines regarding the separation of medical and commercial roles.

Content moderation may be applied by an AI system as described herein, to ensure that messages are compliant with regulations, such as healthcare regulations. Such content moderation may be combined with the messaging systems as described herein, to ensure that appropriate content is sent to the appropriate recipient. For example, these systems may ensure that medical information is only sent to a HCP or HCP staff. These systems may also ensure that physical samples of a medical product are only sent to a HCP who has the appropriate license to receive that product. These systems may therefore reduce the risk of information and/or a product being sent to a contact who does not have the requisite credentials, such as a license, to receive that information and/or product.

Preferably, the AI system is capable of one or more of the following, without wishing to be limited by a closed list:
Identifying whether a communication is commercial or medical in nature
Routing messages to appropriate personnel based on content type
Ensuring medical personnel at a pharmaceutical company, or other supplier of healthcare goods and supportive services, remain independent from commercial influence
Maintaining clear separation between promotional and non-promotional content
Logging and tracking all communications for compliance purposes

Additionally, such systems may also protect the privacy of HCPs and/or personnel at companies who provide products and/or services, by masking the contact details (including but not limited to an address, wherein said address may comprise one or more of a telephone number, a social media address, an email address, a voice modality address, a chat address and a video transmission address, and/or other messaging modality address, and/or other information), while still enabling messages to be correctly and quickly routed to the recipient. Optionally, the video transmission address comprises one or more of a social media address, a email address, q chat address or a live streaming video interface.

Furthermore, according to at least some embodiments, the system as described herein comprises a content moderation function, which may be located at a server through which a message or other content passes before being sent to a recipient. The content analysis function preferably analyzes such content for compliance with a content policy. The content policy may be determined according to a healthcare regulation or law. Optionally, the content policy is determined according to an organization controlling a sending user computational device, such that for example the sending user is a member of the organization. If the organization is a sales organization, then the member may be salesperson or sales support staff member. If the organization is a healthcare organization, then the member may be healthcare personnel (HCP).

Optionally, the content analysis function comprises an AI engine for analyzing the content for content moderation. Various non-limiting, exemplary embodiments of such an AI engine may be used alone or in combination. For example, the AI engine may analyze the content according to a natural language processing (NLP) machine learning algorithm and/or according to an image processing machine learning algorithm. The AI engine may comprise a machine learning algorithm selected from the group consisting of a CNN (convolutional neural network), a DBN (deep belief network), a BiLS™ (bilateral long short term memory network), Sentence-Transformers, BERT (Bidirectional Encoder Representations from Transformers), modifications of BERT, another type of RNN, logistic regression models for NLP, Naïve Bayesian models, Generative Pre-Trained Transformer (GPT), and Mixture of Experts (MoE).

Optionally, the AI engine further receives additional information about a sending user and a recipient user, and further analyzes the content according to the additional information. If the AI engine determines that the message being sent is determined to be acceptable under said content policy, then the AI engine permits the message to be transmitted. But if the message is determined to violate the content policy by the AI engine, the AI engine sends the message to the supervisory user computational device for review.

As described herein in various embodiments, preferably the AI engine further logs the content and a result of a review of the content according to the content policy, with the additional information if available, at a data storage as described herein.

The communication systems as described herein assist with the maintenance of records demonstrating compliance, as well supporting compliance with the necessary rules, for example for a corporation as a non-limiting example of an organization. In evaluating a corporation's policies and mechanisms for identifying, reporting, investigating, and remediating potential misconduct and violations of law, those who enforce the laws and regulations in regard to such communications may consider a corporation's policies and procedures governing the use of personal devices, communications platforms, and messaging applications, including ephemeral messaging applications. Policies governing such applications are preferably tailored to the corporation's risk profile and specific business needs and ensure that, as appropriate and to the greatest extent possible, business-related electronic data and communications are accessible and amenable to preservation by the corporation. The systems and methods as described herein support these various goals.

In combination, these systems not only reduce risk but also increase the reliability of messaging transmission while maintaining appropriate separation between medical and commercial communications. Information may be sent faster and with a greater degree of personalization and/or customization, yet still remain compliant with healthcare regulations and maintain the independence of medical communications from commercial influence.

As a non-limiting example, the system may regulate the interactions between various types of personnel at a pharmaceutical company, and HCPs. The use of a pharmaceutical company is intended as an example only, as any type of company that sells goods and/or supportive services to HCPs (and for example their associated organization) is included. The system as described herein implements specific controls for different types of communications between parties, establishing clear boundaries while enabling necessary interactions for healthcare delivery.

For example, certain types of communication may be permitted. Medical personnel at the pharmaceutical company may operate within a defined scope of scientific and educational responsibilities. Their permitted communications may encompass responding to unsolicited medical information requests from HCPs and facilitating discussions about company-sponsored research opportunities with qualified investigators. They may provide scientific, medical, and research-related information within approved parameters, while sharing aggregated healthcare provider insights that exclude specific prescribing details. Their educational role may extend to conducting non-promotional medical education activities and training contracted speakers on approved medical content when requested. Additionally, they may serve as the primary channel for responding to safety-related inquiries and adverse event reports.

Commercial personnel and sales staff at the pharmaceutical company preferably focus on approved promotional and business activities within strict regulatory boundaries. Their communications center on presenting approved promotional materials about products and discussing approved product indications and usage. They handle business aspects including sharing pricing and contracting information within approved parameters and coordinating logistics for approved educational programs. When necessary, they may make brief introductions of medical personnel to HCPs and provide approved product literature and marketing materials. They also maintain responsibility for coordinating sample distribution according to established protocols.

Joint communications between medical and commercial personnel at the company, with an HCP, preferably require careful oversight and pre-approval to maintain appropriate boundaries while enabling necessary collaboration. These permitted interactions include initial introductory meetings with new healthcare facilities and logistical coordination for approved medical education programs. Brief transitional interactions are allowed when transferring HCP inquiries between personnel types, as are pre-approved customer needs assessments. Additional collaborative activities may include authorized quality assurance reviews and compliance training sessions.

Commercial personnel and sales staff may face specific restrictions designed to maintain the independence of medical communications. They may be prohibited from requesting medical personnel to provide off-label product information or assist in overcoming prescribing barriers. Information barriers may prevent them from seeking details about ongoing clinical trials or research programs, and they preferably cannot attempt to influence medical content development or modification. To maintain clear role separation, they preferably cannot direct or appear to direct medical personnel activities, engage in detailed discussions about ongoing research, or participate in the development of medical/scientific content. They may be restricted from requesting specific HCP prescribing information from medical personnel and preferably must avoid discussing unapproved products or indications.

Medical personnel preferably operate under complementary restrictions to maintain their scientific independence. For example, they may be prohibited from participating in sales strategy meetings or engaging in pricing or contracting discussions. To maintain appropriate boundaries, they cannot share specific HCP prescribing information or discuss details of unapproved medical content with commercial personnel. Their role separation preferably extends to excluding them from promotional planning activities, commercial messaging, or marketing advice. They must preferably avoid direct promotional activities and cannot assist in overcoming sales obstacles.

The system's flexibility allows for customization based on varying requirements and contexts. These controls are designed to be configurable to accommodate variations in regional regulatory requirements, organizational structure and policies, product-specific requirements, healthcare provider type and specialty, communication channel characteristics, and risk level of specific communication types.

Comprehensive logging preferably creates a detailed audit trail of all communications. The system maintains records of time and date of communication, sender and recipient identification and roles, communication channel used, content classification results, any override or exception authorizations, resolution of any flagged communications, and an audit trail of any modifications or redirections.

According to at least some embodiments, the system preferably implements one or more technical mechanisms to enforce communication boundaries between medical and commercial personnel, ensuring regulatory compliance while maintaining efficient information flow. These mechanisms operate at various levels of the communication system, from message creation to delivery and archival. These preferably include content moderation, routing control and so forth, as described herein.

The system preferably implements continuous monitoring of communication patterns and content flows, in a real-time monitoring and intervention system. This monitoring system may comprise one or more AI models as described herein, and may incorporate one or more of the following technologies and implementations:

Pattern recognition algorithms to identify potential attempts to circumvent communications boundaries. For example, if a series of messages between commercial and medical personnel shows increasing frequency or unusual timing patterns, the system flags this for compliance review.

Content similarity analysis to detect potential replication of restricted communications across different channels. When similar content appears in multiple messages through different routes, the system analyzes whether this represents an attempt to bypass restrictions.

Behavioral analytics to identify unusual communication patterns that might indicate attempts to circumvent role-based restrictions. For instance, if commercial personnel frequently initiate conversations that are then continued by medical personnel, the system flags this pattern for review.

Preferably the system features automated documentation and audit trail generation, to maintain comprehensive documentation of all communications through automated logging and audit trail generation. This documentation system preferably creates detailed records which may include one or more of the following:

Communication metadata capturing timestamp, sender, recipient, channel, and routing information. For example, when a medical inquiry is redirected from commercial to medical personnel, the system logs the original sender, all intermediary handlers, and final recipient.

Content versioning that tracks any modifications to messages during transmission or review. If a message is modified during compliance review, the system maintains both original and modified versions with associated justification for changes.

Access logs recording all attempts to view, modify, or transmit restricted content, including failed attempts and system interventions. When a user attempts to access content outside their role permissions, the system logs the attempt and the basis for rejection.

The system also preferably provides secure APIs and integration points for external systems while maintaining communication boundaries. These integration capabilities may include one or more of the following:

Secure message transformation services that ensure proper content handling across different communication platforms. When messages move between internal and external systems, content is automatically analyzed and reformatted to maintain compliance.

Role-based API access controls that extend internal permission systems to external integrations. External systems must provide appropriate role credentials to access specific API endpoints or data types.

Compliance validation interfaces that enable real-time checking of external communications against internal policies. Before allowing external transmission, the system validates message content and routing against all applicable rules and restrictions.

As noted above, these systems may be implemented in a variety of ways, including with regard to rules-based systems and AI supported systems. As a non-limiting example, the AI system may implement a multi-layered architecture specifically designed to maintain and enforce the separation between medical and commercial communications through sophisticated machine learning models and rule-based systems.

According to at least some embodiments, the system provides comprehensive communication capabilities, including advanced audio and video messaging features. Users can record and share audio or video messages directly within chat conversations. For voice communications, the system implements a sophisticated bridge call system where representatives can call using their virtual number, which then connects to their actual number. When the representative answers, the system calls the healthcare professional's (HCP's) virtual number, effectively bridging the representative's actual phone and the HCP's virtual number through the representative's virtual number. Users can also set up custom voicemail messages.

Video call functionality preferably includes several enhanced features. When participants join a video call, they first enter a lobby where they can view promotional material while waiting. The host must accept participants before they can enter the actual meeting. During video calls, participants can blur their backgrounds and record the meeting. The system supports calendar integration, allowing synchronization with various calendar software solutions to and from the system.

The system preferably provides extensive reporting capabilities, including event territory analysis, time analysis, call tracking, opt-in/opt-out monitoring, and days analysis reports. Users can access active roster reports and compliance keyword reports. All report data can be exported, and users can create personalized dashboards based on their specific needs.

Event management is preferably fully supported, allowing users to create events with detailed information including event name, type, venue, time, and date. Users can add attendees and track expenses, with integration to expense management systems and CRMs. The system includes an AI assistant crafted to support HCPs, patients, and sales representatives with their inquiries, ensuring accuracy and reliability for healthcare needs.

Custom out-of-office (OOO) messaging is preferably supported, allowing users to write personalized OOO messages. The system also supports “bring your own number” functionality, where users can register their preferred number through profile verification. Administrative capabilities include an “act as” function, allowing administrators to access user data and modules based on client-set permissions.

Dynamic templates enable users to incorporate variable elements like time, date, and text when sending messages to contacts. The system supports shared number workflows, where incoming messages to a shared number can be redirected through pooling or queue mechanisms. Administrators can transfer message conversations, and known contacts can be handled based on recent activity or territory alignment.

Contact management features include comprehensive address details (primary and secondary), contact timeline tracking, opt-in/opt-out capabilities, and document sharing. Contacts can be marked as favorites, and users have quick access to communication options like calling, chat, and video calls. When a user is out of office, territory coverage can be managed through a covering representative system, with permissions based on client requirements.

Content moderation features include highlighting pre-configured words based on client configuration, logging restricted words for dashboard display, and message reporting capabilities. Users can report messages with reasons, leading to soft deletion or highlighting based on client configurations. The scheduling system includes meeting rejection notifications and rescheduling features with automated new video meeting link distribution.

The system supports enhanced profile management, allowing users to modify time zones and language preferences, and share QR codes. A sophisticated flow builder enables client administrators to create and edit workflows through the admin UI, with scheduled or triggered execution options. Actions can include record creation/updates, record activation/deactivation, communication initiation, and various other functions based on defined conditions.

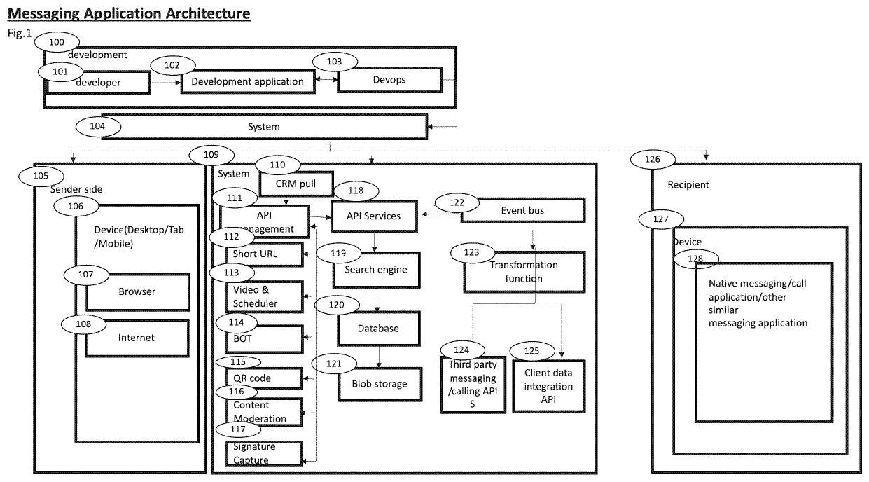

Turning now to the drawings, FIG. 1 shows a non-limiting, exemplary system for secure healthcare communication. As shown, the system features a developer environment 100 for developing one or more applications for an application system 104. Developer environment 100 preferably features a developer 101, interacting with a development application 102 and supported by one or more developer tools 103. The output from developer environment 100 supports the addition of functionalities to application system 104.

Application system 104 supports communication between a sender side 105 and a recipient side 126, through a server side 109. Preferably no additional software is installed at sender side 105 and/or recipient side 126, although one or more software modules are installed at server side 109 to support communication.

Sender side 105 preferably comprises any suitable computational device 106 that is capable of operating a message application 107. Optionally message application 107 is any suitable, standard, off the shelf message application as described herein, or alternatively may comprise any suitable browser based and/or mobile phone operating system based application. Message application 107 is preferably able to communication with server side 109 through for example a regular messaging channel connection, including without limitation a messaging app identifier and/or a mobile device telephone number. Message application 107 may also comprise a web browser as shown. Communication may be supported by any suitable communication channel, including without limitation any type of computational network such as the internet, separate cellular network communication and so forth, shown as internet 108.

Server side 109 preferably exchanges messages with message application 107, for example to send to another such message application, of the same or a different type, such as application 128 operated by a recipient computational device 126 as shown. Again, any suitable messaging channel connection (as a separate network) may be used to connect application 128 and server side 109, including without limitation a telephone network, a cellular network, an internet, and/or a messaging modality company server connected through any of a telephone network, a cellular network and the internet. Preferably both message application 107 and application 128 are multi-purpose such applications, rather than being installed for the specific purpose of communication through server side 109. Such applications may comprise messaging applications, calling (voice communication) applications and so forth.

Non-limiting examples of such messaging applications include WhatsApp, Telegram, Signal, Facebook Messenger, WeChat, Viber, Line, Google messaging, Instagram for business, Apple chat, native SMS application functionality, native or live chat functionality and the like. These applications represent third-party communication services that: are independently installed and maintained on user devices; operate using their own distinct communication protocols, authentication methods, and security measures; require separate user accounts and access credentials independent from the main system; function as standalone applications with their own feature sets and capabilities; and process messages through their own proprietary networks and servers.

In each case, rather than connecting directly through the servers or communication channels normally associated with each such application, both message applications 107 and 128 connect with an address or other identifier which appears to be a normal identifier for each such message application 107 or 128, but instead are directed to connect through server side 109, which mediates the connection. This mediated connection allows the system to maintain separate authentication and security protocols; apply content moderation and policy compliance checks; mask actual user addresses and identifiers; route messages through appropriate channels while maintaining regulatory compliance; log and track communications across different messaging platforms; ensure appropriate separation between medical and commercial communications regardless of the messaging platform used.

The messaging applications are considered ‘not native’ to the system because they:
1. Are added to the system post-deployment through separate installation processes
2. Maintain their own independent operational frameworks
3. Use proprietary protocols and communication methods distinct from the main system
4. Require separate user authentication and access management
5. Can function independently of the main system's core operations
6. Process communications through their own distinct networks and servers before being mediated by the system's server”

As noted previously, such an address may comprise one or more of a telephone number, a social media address, an email address, a voice modality address, a chat address and a video transmission address, and/or other messaging modality address, and/or other information. Optionally, the video transmission address comprises one or more of a social media address, a email address, q chat address or a live streaming video interface.

Server side 109 comprises an API management 111, which receives various types of communication, including but not limited to such exchanged messages. API management 111 then determines the correct API to which the communication is to be passed, and then passes the communication to one or more API services 118. API services 118 may pass the communication directly to an external API (not shown), and/or may pass the communication to a search engine 119 and/or an event bus 122.

Search engine 119 may comprise one or more AI models and/or be configured as an AI engine as described herein. Search engine 119 may determine which external API is to receive the communication according to one or more models and/or machine learning algorithms. For example, search engine 119 may analyze the content of the communication and determine that it relates to an emergency, as well as whether the communication is being received from a patient or other subject in the emergency, or from a healthcare provider. Search engine 119 may also determine that the communication relates to a request for information, to a connection to a particular healthcare provider or patient, to a request for an appointment and so forth. Search engine 119 may retrieve required information, such as the information needed to make a further connection and/or to answer the request for information, from a database 120. Such requests may then be logged in a physical storage, such as a blob storage 121.

Turning now to event bus 122, the communication may be further passed to a 120 transformation function 123, which determines whether the communication is to be passed to one or more 121 third party messaging APIs 120 and/or to a client data integration API 125. One or more third party messaging APIs 124 may for example support communication with message application 107 and application 128, for example as one or more of WhatsApp, Telegram, Signal, Facebook Messenger, WeChat, native SMS application functionality and the like. Client data integration API 125 preferably supports communication with an external client data source and/or software. The role of “client” may for example be performed by any suitable stakeholder, including but not limited to healthcare professionals, office staff, and/or external support staff. External support staff may include staff at diagnostic laboratories, other medical centers, and staff at providers of goods and services to healthcare professionals, including without limitation sales staff, pharmacists, medical device specialists, scientists and the like.

Server side 109 may also comprise a URL shortening module 112, for easier transmission of links to chats, video calls, information and so forth. Server side 109 may also comprise a video call management module 113, which enables video calls to be scheduled and then supports communication through such video calls. Server side 109 may also comprise a bot 114 for supporting automated communication, for example through a messaging function as described herein.

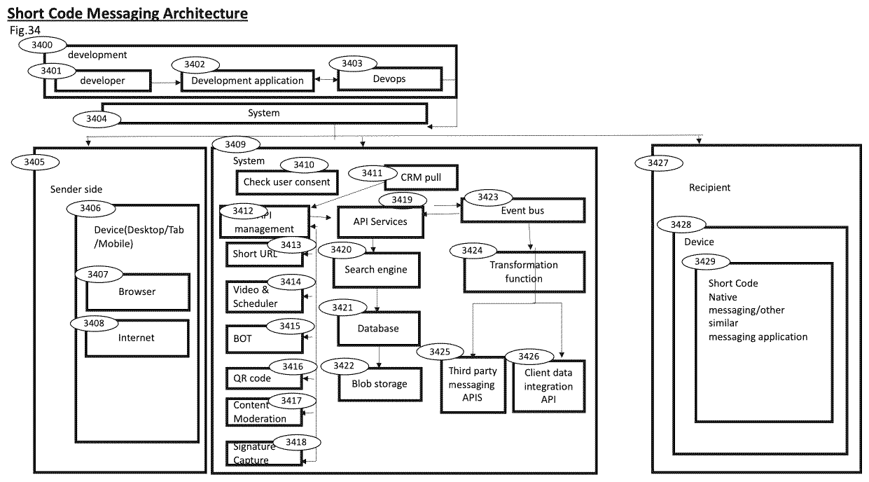

FIG. 34 relates to a non-limiting, exemplary architecture for short code messaging, which is similar to that of FIG. 1, but is specifically directed to mobile telephony. In particular, a mobile telephony device 3428 is able to operate a messaging app 3429, which is able to provide Short Code Native messaging and/or another similar messaging application.

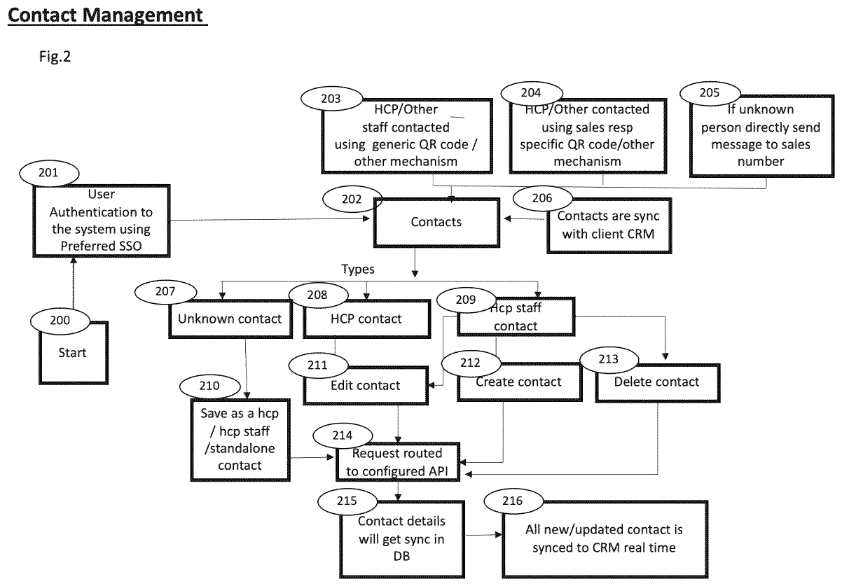

FIG. 2 relates to a non-limiting, exemplary method for contact management. The method may be operated by the system of FIG. 1 as a non-limiting example. As shown, the method 200 begins with user authentication to the system, for example using SSO (single sign on) technology. Next, the user is able to review contacts 202, optionally filtering them by type. Some non-limiting examples of types of contacts to be filtered, and then optionally updated and/or reviewed, include an unknown contact 207, which may then be saved as a HCP (healthcare provider) or HCP staff at 210. A HCP contact 208 may be edited (updated, changed, created and/or deleted) at 207. A HCP staff contact 209 may be edited at 211, created at 212 and/or deleted at 213. Optionally, creation of a new HCP contact 208 may be performed by saving/associating unknown telephone numbers and/or message modality addresses (if not telephone numbers) that are flowing into the system. The contact may be a HCP or a non-HCP contact.

After any of these actions 210-213 are performed, then at 214, the request for the results of any of these contact management actions to be saved in the database is preferably sent to the configuration API. The contacts database is then updated at 215. The CRM is also preferably synchronized at 216.

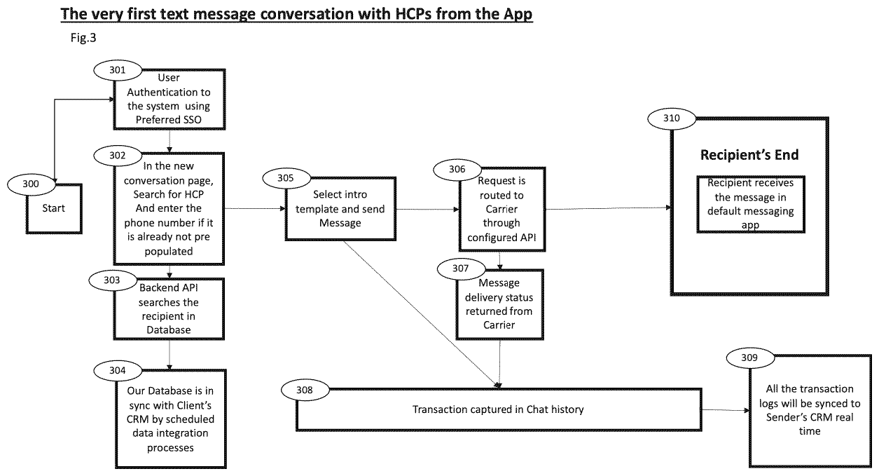

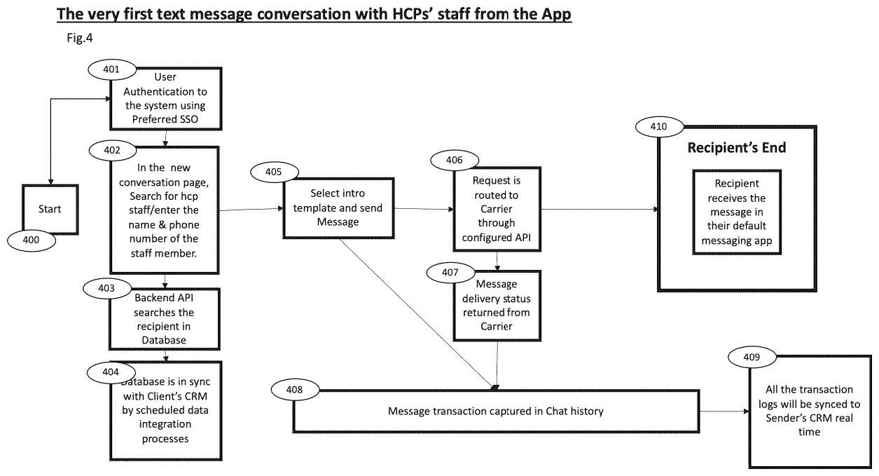

FIG. 3 shows a non-limiting, exemplary method for initiating communication with a healthcare professional. As shown, a healthcare provider, office staff member, external support staff member or other user initiates communication at 300, for example by logging into a software interface. For the purpose of description only, the user is assumed to be an external support staff member attempting to contact an HCP (healthcare professional). The user in this non-limiting example is the sender of a message and the HCP is the recipient. The user is then invited to authenticate at 301, for example by using an SSO (single sign-on) method, which may be performed through an existing email, social media or other user account. At 302, the user may be shown a landing page and/or may employ a voice command to indicate which healthcare professional is to be contacted. If the phone number or other identifier of that healthcare professional is not available, then preferably the user enters it. Although the phone number is a preferred identifier for the healthcare professional, optionally a social media account identifier or other identifier may be used. Where suitable, such an identifier may be substituted for “phone number” when the latter is discussed herein.

At 303, the backend API receives the request and searches for the correct healthcare provider information, for example in the previously described server database. The healthcare provider information may include for example contact details of the healthcare provider to be contacted, including but not limited to phone number and/or social media contact information. Optionally such contact information is for a “cut-out” communication modality, in which the private contact details of the HCP are kept private, while contact information that the server supports is provided. At 304, the database is preferably synchronized with the CRM (customer relationship management) database of the external support staff company or organization by scheduled data integration processes. For example, such an synchronization may be performed through the client data integration API of FIG. 1 (not shown).

At 305, an introduction or other suitable template is selected, for initiating a new message, for example to the selected HCP as recipient. If this is the first time that the user has contacted the recipient using this message modality, or optionally any modality, then preferably an introduction template is selected. The message is then sent. At 306, the request to send the message is routed as necessary to transmit it. For example, for an SMS, the request is preferably routed to a cellular network communication provider, such as a carrier, through the previously described configured API.

At 307, the delivery status is returned from the carrier or other message communication provider. Optionally, if the delivery status is a failed delivery, then the message may be sent through a different messaging modality and/or repeated through the same messaging modality. This option applies to all messaging methods and systems as shown herein, and for all embodiments and Figures as shown herein. The message transaction is preferably captured in a chat history of communication between that authenticated user and the specific recipient at 308.

At 309, the recipient receives the message in their default messaging app for that type of communication, such as an SMS message app on a mobile communication device for example. Preferably the default is set in advance and is stored in the previously described CRM.

At 309, all the transaction logs are synchronized to the customer's CRM in real time or in a batch process.

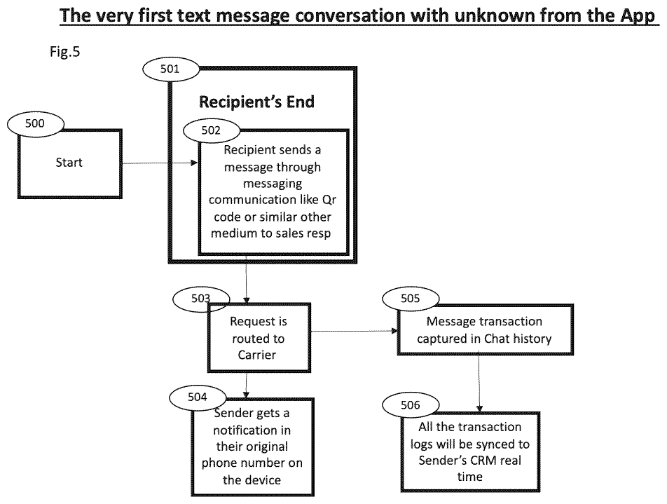

FIG. 4 shows a non-limiting, exemplary method for initiating communication with a staff member of an office of a healthcare professional by an external support staff member. For example, optionally the method shown in FIG. 3 is performed with a healthcare professional as recipient, while the staff interactions may be performed through the method shown with regard to this drawing. The external support staff member initiates communication at 400, for example by logging into a software interface, and then by authenticating at 401, as previously described.

At 402, the user may be shown a landing page and/or may employ a voice command to indicate which member of the office staff of a healthcare professional is to be contacted. If the phone number or other identifier of that office staff member is not available, then preferably the user enters it. Although the phone number is a preferred identifier for the office staff member, optionally a social media account identifier or other identifier may be used. Where suitable, such an identifier may be substituted for “phone number” when the latter is discussed herein.

At 403, the backend API receives the request and searches for the correct office staff member information, for example in the previously described server database. The office staff member information may include for example contact details of the staff member to be contacted, including but not limited to phone number and/or social media contact information. Optionally such contact information is for a “cut-out” communication modality, in which the private contact details of the staff member are kept private, while contact information that the server supports is provided. At 404, the database is preferably synchronized with the CRM (customer relationship management) database of the external support staff company or organization by scheduled data integration processes. For example, such an synchronization may be performed through the client data integration API of FIG. 1 (not shown).

At 405, an introduction or other suitable template is selected, for initiating a new message, for example to the selected office staff member as recipient. If this is the first time that the user has contacted the recipient using this message modality, or optionally any modality, then preferably an introduction template is selected. The message is then sent. At 406, the request to send the message is routed as necessary to transmit it. For example, for an SMS, the request is preferably routed to a cellular network communication provider, such as a carrier, through the previously described configured API.

At 407, the delivery status is returned from the carrier or other message communication provider. The message transaction is preferably captured in a chat history of communication between that authenticated user and the specific recipient at 408.

At 409, all the transaction logs are synchronized to the customer's CRM in real time or in a batch process.

At 410, the recipient receives the message in their default messaging app for that type of communication, such as an SMS message app on a mobile communication device for example. Preferably the default is set in advance and is stored in the previously described CRM.

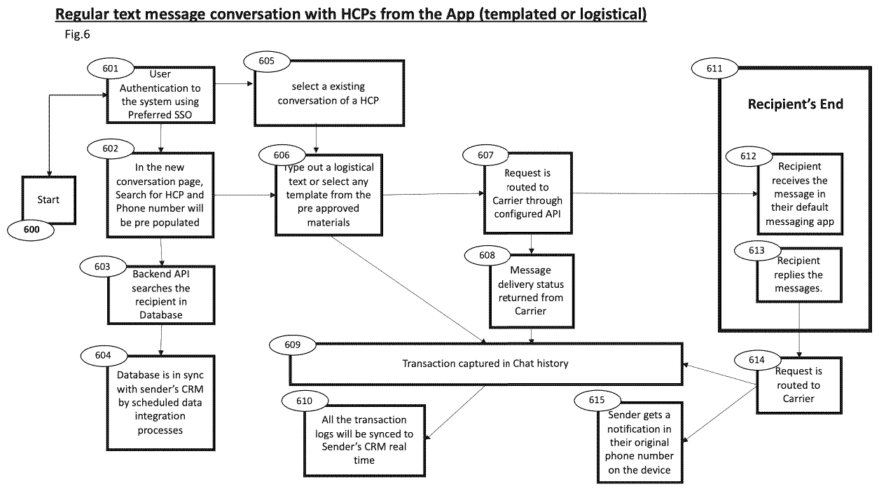

FIG. 5 shows a non-limiting, exemplary method for initiating communication with a contact that was not previously known. In this non-limiting example, the recipient designated in other flows, such as HCP or HCP staff member, initiates the process by sending a contact to a sales representative, or other representative or personnel of a company or organization. As shown, the process begins at 500. At 501, a recipient decides to initiate communication as described above. At 502, the recipient sends a message through a messaging communication modality. For example, the communication may be initiated by scanning a QR code as described herein. The message is sent a sales representative or other personnel.

At 503, the request to send the message is routed as necessary to transmit it. For example, for an SMS, the request is preferably routed to a cellular network communication provider, such as a carrier, through the previously described configured API.

At 504, the recipient of the message (the sender in other flows as described herein) receives the message from the unknown contact. The message transaction is preferably captured in a chat history of communication between the recipient and sender at 505.

At 506, all the transaction logs are synchronized to the customer's CRM in real time or in a batch process.

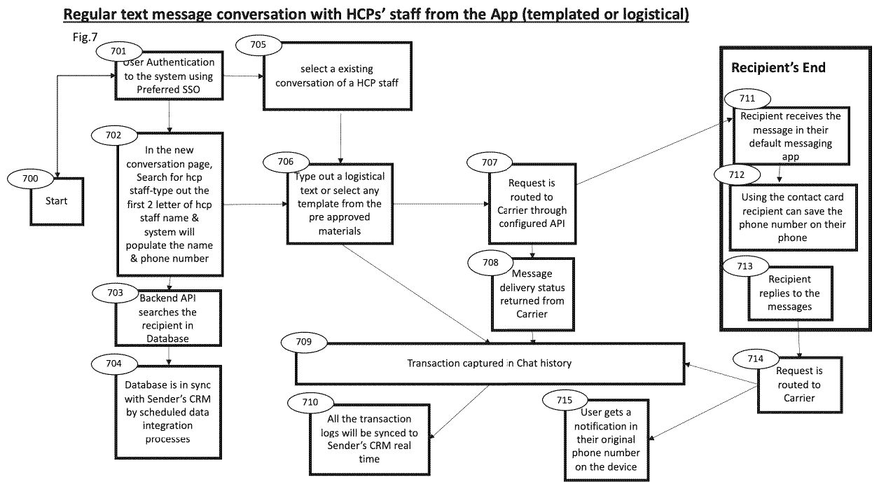

FIG. 6 shows a non-limiting, exemplary method for communication between an external support staff member and a recipient, who could be a healthcare professional for example. In this non-limiting example, the conversation is assumed to be between an HCP and an external support staff member. Preferably, this method is employed after communication is established between the healthcare professional and the external support staff member, whether generally or for this specific communication modality. Optionally, stages 600-604 are similar or identical to stages 300-304 of FIG. 3. An existing conversation with an HCP may optionally be selected to begin, at 604.

At 606, the external support staff member determines the message structure and content, for example from one or more templates, and/or as free-form text, or a combination thereof. For example and without limitation if the healthcare professional has previously requested assistance with a particular good or service, and/or is requesting information, then a suitable template may be selected, for example to request that the healthcare professional call the external support staff member, login to a secure portal, or otherwise perform some action. Alternatively or additionally, the external support staff member may transmit information directly in the message. Optionally the external support staff member may request the HCP to perform some other action such as a booking an appointment.

At 607, the request to send the message is routed as necessary to transmit it as previously described. In this non-limiting example, the message is an SMS message and the request is routed to a suitable carrier.

At 608, the delivery status is returned from the carrier or other message communication provider. The message transaction is preferably captured in a chat history of communication between that authenticated user and the specific recipient at 609.

At 610, all the transaction logs are synchronized to the customer's CRM in real time or in a batch process as previously described.

At 611, the recipient receives the message, for example in their default messaging app for that type of communication, such as an SMS message app on a mobile communication device for example, shown at 612. In this non-limiting example, the recipient is assumed to be a healthcare professional. Again, preferably the default is set in advance and is stored in the previously described CRM. At 613 the recipient replies, whether because the message content requested a reply or independently. At 614, the request is routed through the carrier for an SMS message. At 615, the user receives a notification, which is routed to that user according to the previously entered phone number or other identifier. The notification may be sent by the backend as an SMS message, or alternatively may be sent as some other type of message, according to the user contact configuration.

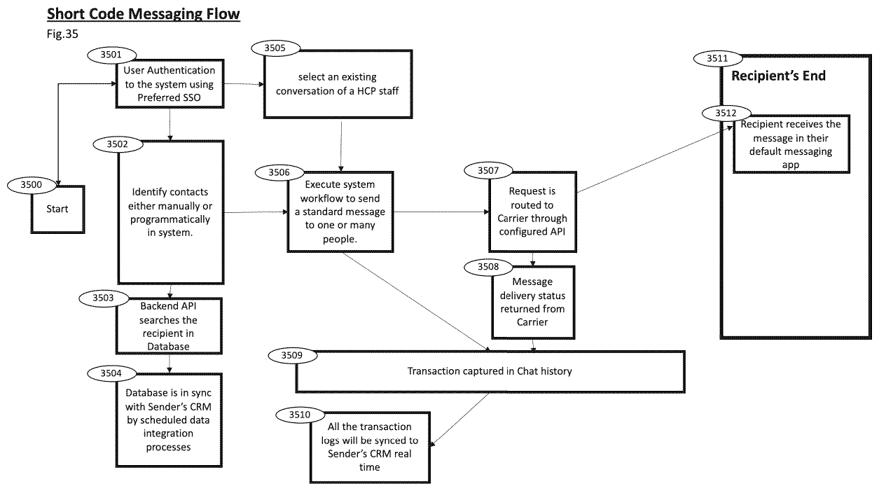

FIG. 35 relates to a non-limiting, exemplary flow for short code messaging through mobile telephony.

FIG. 7 shows a non-limiting, exemplary method for communication between a staff member of an office of a healthcare professional and a patient or an external support staff member. In this non-limiting example, the communication is between the staff member of the healthcare professional and the external support staff member. Optionally, stages 700-704 are similar or identical to stages 400-404 of FIG. 4.

Optionally, at 705, the message may be sent as part of an existing conversation with a HCP staff contact, for example as part of a message thread or in reply to an already sent message.

At 706, the external support staff member determines the message structure and content, for example from one or more templates, and/or as free-form text, or a combination thereof. For example and without limitation if the external support staff member wishes to transmit information or alternatively wants the HCP staff member to contact them, then a suitable template may be selected, for example to request that the HCP staff member call the external support staff member, the HCP and/or the office; login to a secure portal, or otherwise perform some action. Alternatively or additionally, the external support staff member may send information directly in the message.

Stages 707-714 may be similar or identical to stages 607-614 of FIG. 6. At 715, optionally the external support staff member receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

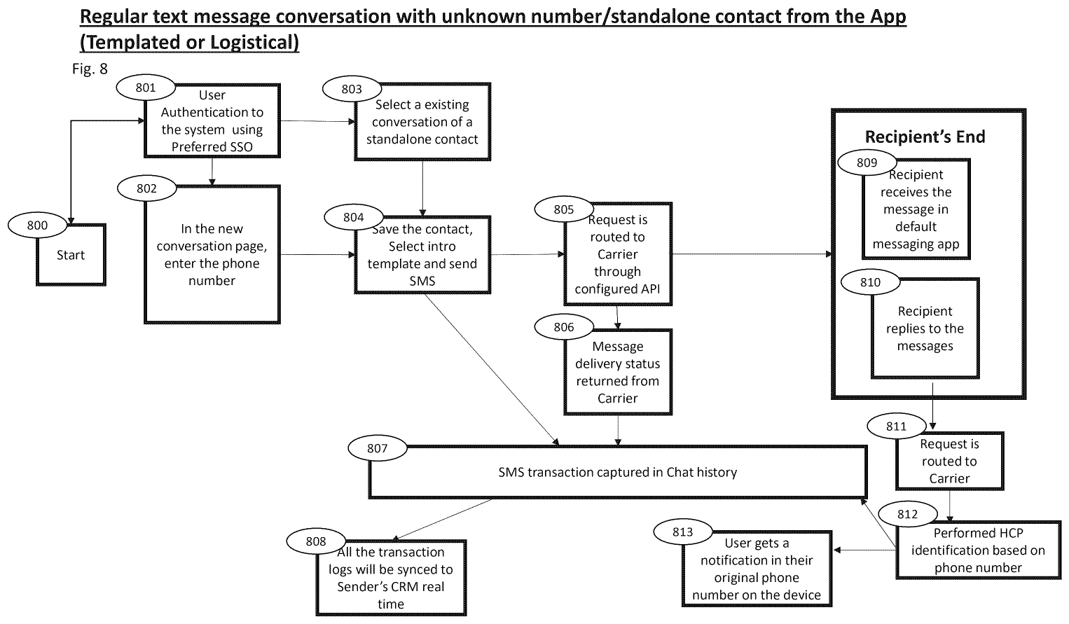

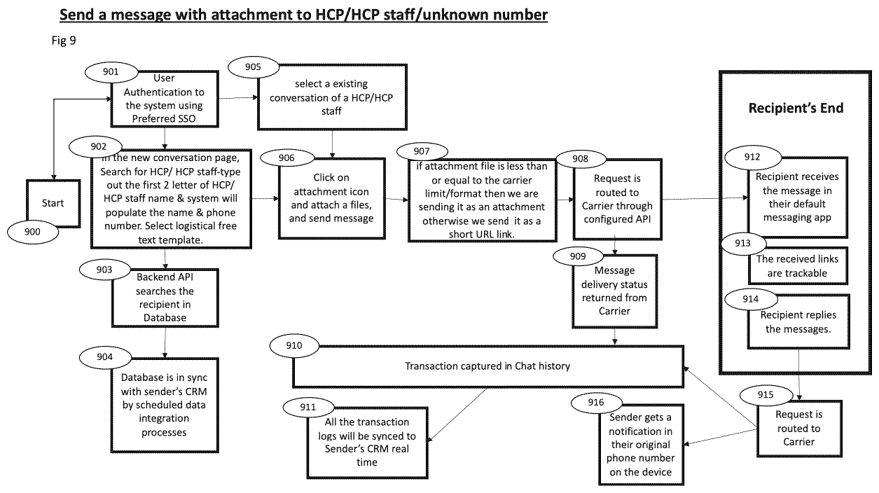

FIG. 8 shows a non-limiting, exemplary method for communication with a previously unknown contact.

The external support staff member initiates communication at 800, for example by logging into a software interface, and then by authenticating at 801, as previously described.

At 802, the user may be shown a new conversation page and/or may employ a voice command to indicate which member of the office staff of a healthcare professional is to be contacted. If the phone number or other identifier of that office staff member is not available, then preferably the user enters it. Although the phone number is a preferred identifier for the office staff member, optionally a social media account identifier or other identifier may be used. Where suitable, such an identifier may be substituted for “phone number” when the latter is discussed herein.

At 803, the user may decide to select an existing conversation with a standalone contact, for example as part of a message thread or in reply to an already sent message. The backend API receives the request and searches for the correct office staff member information, for example in the previously described server database. The office staff member information may include for example contact details of the staff member to be contacted, including but not limited to phone number and/or social media contact information. Optionally such contact information is for a “cut-out” communication modality, in which the private contact details of the staff member are kept private, while contact information that the server supports is provided.

At 804, the contact is saved. The user then selects an “intro” template for making an introduction, and then sends a message, such as an SMS message for example, to the desired contact. For example, the user, such as an external support staff member, determines the message structure and content, for example from one or more templates, and/or as free-form text, or a combination thereof. For example and without limitation if the external support staff member wishes to transmit information or alternatively wants the unknown contact to contact them, then a suitable template may be selected, for example to request that the unknown contact call the external support staff member; login to a secure portal, or otherwise perform some action. Alternatively or additionally, the external support staff member may send information directly in the message. Optionally such information is limited in content because the contact is unknown, and hence it is not possible to determine whether certain content is permitted to be sent.

At 805, the request is routed through the appropriate message carrier, through the configured API. At 806, the message delivery status is returned from the carrier. At 807, the SMS (or other message) transaction is preferably captured in the chat history. At 808, the transaction logs are preferably synchronized with the CRM (customer relationship management) database of the external support staff company or organization by scheduled data integration processes, which may optionally include real time synchronization. For example, such a synchronization may be performed through the client data integration API of FIG. 1 (not shown).

At 809, the recipient receives the message in the default message app. At 810, the recipient replies to the message, for example using the messaging modality as previously described. At 811, the request to send the message is routed as necessary to transmit it. For example, for an SMS, the request is preferably routed to a cellular network communication provider, such as a carrier, through the previously described configured API.

At 812, optionally an identification process is performed to identify the contact, for example according to phone number. For example, the telephone number may be associated with a HCP or a HCP staff member, or with a medical organization such as a hospital for example.

At 813, optionally the user, such as a member of external support staff for example, receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

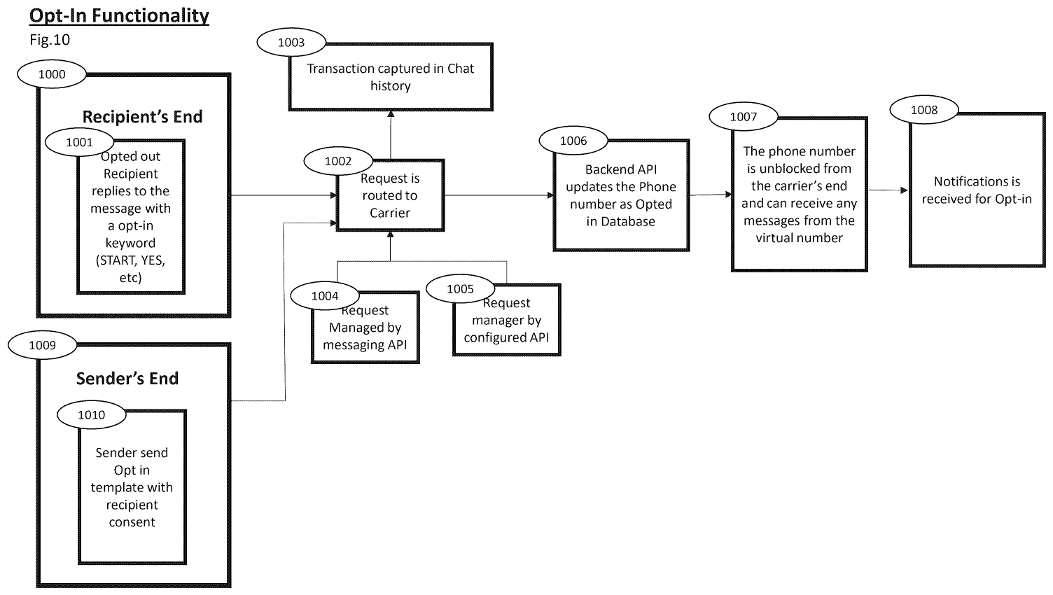

FIG. 9 shows a non-limiting, exemplary method for sending a message with an attachment to a HCP contact and/or a HCP staff contact. As shown, a healthcare provider, office staff member, external support staff member or other user initiates communication at 900, for example by logging into a software interface. For the purpose of description only, the user is assumed to be an external support staff member attempting to contact an HCP. The user in this non-limiting example is the sender of a message and the HCP is the recipient. The user is then invited to authenticate at 901, for example by using a SSO method, which may be performed through an existing email, social media or other user account.

At 902, if a new conversation is to be started (that is, a conversation or message that is not based on a previously sent message), then the user enters the new conversation page. Optionally, a previous conversation may be defined as one that is performed according to a single messaging modality, although optionally and alternatively, a previous conversation may also combine a plurality of communication modalities, including a plurality of messaging modalities and/or other communication modalities. In that page, the user searches for a contact from a HCP/HCP staff. As a non-limiting example, the user may enter the first two letters of the name of the contact, and/or of the name of the office or organization of the contact. The system may then populate the name, phone number and optionally other contact information such as an email address and/or cellular phone number. The user then preferably selects the logistical free text template. A logistical free text template is a template which enables a freely written or created text message/attachment to be sent from the new conversion screen, or from a screen related to an existing conversation.

Next, at 903, the backend API receives the request and searches for the contact information, for example in the previously described server database. The contact information may include for example contact details, including but not limited to phone number and/or social media contact information. Optionally such contact information is for a “cut-out” communication modality, in which private contact details are kept private, while contact information that the server supports is provided. At 904, the database is preferably synchronized with the CRM (customer relationship management) database of the external support staff company or organization by scheduled data integration processes. For example, such a synchronization may be performed through the client data integration API of FIG. 1 (not shown).

Optionally, at 905, the message may be sent as part of an existing conversation with a HCP staff contact, for example as part of a message thread or in reply to an already sent message.

At 906, the user preferably selects one or more message files and attaches the file(s) to the message. For example and without limitation, the file(s) may be attached to an SMS message. At 907, the size of the attachment(s) is considered. If the size is below the limitation for a particular message channel and/or carrier, then the attachments are sent attached to the message. Otherwise, the attachment(s) may be saved to an online cloud service, in which case a short URL link is sent with the message instead.

At 908, the request to send the message is routed as necessary to transmit it. For example, for an SMS, the request is preferably routed to a cellular network communication provider, such as a carrier, through the previously described configured API.

At 909, the delivery status is returned from the carrier or other message communication provider. The message transaction is preferably captured in a chat history of communication between that authenticated user and the specific recipient at 910. At 911, all the transaction logs are synchronized to the customer's CRM in real time or in a batch process.

At 912, the recipient receives the message in their default messaging app for that type of communication, such as an SMS message app on a mobile communication device for example. Preferably the default is set in advance and is stored in the previously described CRM. At 913, if the attachment(s) are instead provided through one or more links, the recipient clicks on the one or more links to receive the attachment(s). This action is preferably trackable. Otherwise, the recipient is preferably able to view the attachment(s) directly from, or by downloading the attachment(s) from, the message. At 914, optionally the recipient replies to the message. If so, then the request is routed through the carrier at 915.

At 916, optionally the user receives a notification, which is routed to that user according to the previously entered phone number or other identifier. The notification may be sent by the backend as an SMS message, or alternatively may be sent as some other type of message, according to the user contact configuration. If there are multiple recipients of the notification, then optionally different messaging modalities may be used.

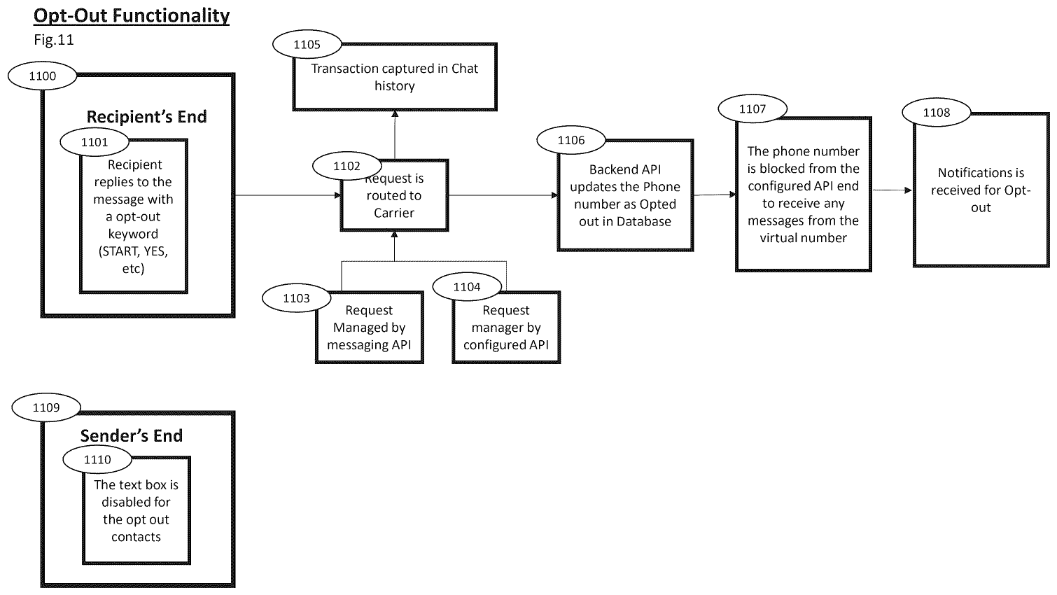

FIG. 10 shows a non-limiting, exemplary method for an opt-in to initiate communication through the system. Such a method may be performed if for example the recipient opted-out accidentally and/or if the recipient has now decided to opt back in to such communication, and/or if the sender organization requests explicit opt-in to communicate further. In this non-limiting example, the opt-in is requested by a recipient, which is assumed to be a device connected to a patient and/or an HCP or HCP staff member. The sender is assumed to be a healthcare professional and/or staff member, and/or an external support staff member. At 1000, the recipient receives a message as previously described. At 1001, the recipient replies to the message sent as previously described with an opt-in request, which may for example comprise a suitable keyword (START, YES, etc).

At 1002, the opt-in request message is routed as necessary to transmit it as previously described. In this non-limiting example, the message is an SMS message and the request is routed to a suitable carrier. The carrier then routes it to the backend server, as the virtual number is associated with the backend server. The message transaction is preferably captured in a chat history of communication between that authenticated user and the specific recipient at 1003.

At 1004, the request is managed by the messaging API. At 1005, the request is managed by the configured API.

At 1006, the Backend API updates the phone number as an “opt in” in the relevant database, so that further requests from the sender to send a message to that phone number and/or other communication modality may be supported. For example, at 1007, the opted out phone number is unblocked from the carrier's end and is now able to receive any SMS messages from the virtual number. At 1008, a notification is received by the recipient in regard to the opt-in.

At 1009, the sender may also receive the opt-in notification. At 1010, if permitted by client configuration, the sender may send an opt-in invitation template to the opted-out recipient. Even after receiving this template, the recipient preferably must still actively respond with an opt-in keyword to resume communications.

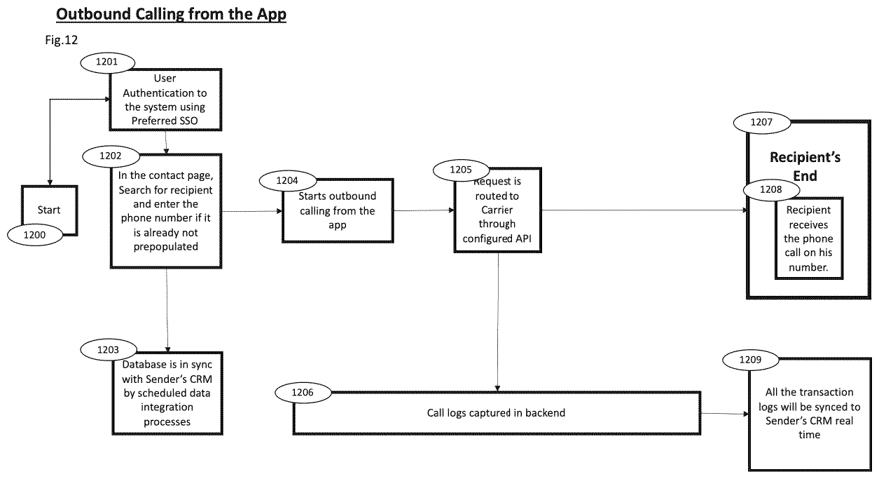

FIG. 11 shows a non-limiting, exemplary method for an opt-out to further communication through the system. In this non-limiting example, the opt-out is requested by a recipient, which is assumed to be device connected to an HCP and/or a patient. The sender is assumed to be an external support staff member and/or a healthcare professional and/or staff member. At 1100, the recipient receives the message, and replies to the message sent as previously described with an opt-out request at 1101, which may for example comprise a suitable keyword (STOP, END, etc).

At 1102, the opt-out request message is routed as necessary to transmit it as previously described. In this non-limiting example, the message is an SMS message and the request is routed to a suitable carrier. The carrier then routes it to the backend server, as the virtual number is associated with the backend server. At 1103, the request is managed by the messaging API. At 1104, the request is managed by the configured API. The message transaction is preferably captured in a chat history of communication between that authenticated user and the specific recipient at 1105.

At 1106, the Backend API updates the phone number as an “opt out” in the relevant database, so that further requests from the sender to send a message to that phone number and/or other communication modality are preferably blocked. For example, at 1107, the opted out phone number is blocked from the carrier's end to receive any SMS messages from the virtual number. The opt-out may be confirmed at 1108. At 1109, the sender and/or others in the organization are notified of the opt out.

At 1110, the sender attempts to send a message to the opted-out identifier, such as an opted-out phone number in this non-limiting example. However, preferably the text box is disabled for opt-out contacts and/or an error message appears, if the sender attempts to send the message to the opted-out identifier.

FIG. 12 shows a non-limiting, exemplary method for outbound voice communication from an external support staff member to an HCP or an HCP staff member. The external support staff member may be a sales representative for a pharmaceutical or other medical supply company. Optionally, stages 1200-1203 are similar or identical to stages 300-304 of FIG. 3.

At 1204, the user, which in this example is a healthcare professional, preferably starts outbound calling from the app to a particular patient (recipient) phone number and/or synchronous voice communication modality. The latter may include but is not limited to WhatsApp, Skype, WeChat and the like. The voice communication modality may also comprise video where supported by the communication modality selected.

At 1205, the request is routed to a suitable carrier and/or other communication channel through a configured API. For example, the carrier may comprise a telephone and/or cellular network provider. Other suitable communication channels may be selected according to the communication modality, including but not limited to WhatsApp, Skype, WeChat and the like. At 1206, preferably the call logs are captured at the backend, including with regard to initiated calls that are not answered, as well as those in which a conversation occurs.

At the recipient 1207, the recipient receives the synchronous voice communication session initiation request, such that a synchronous voice communication session may begin at 1208. For example, for a call to a phone number of the recipient, the synchronous voice communication session may be a telephone call. For other voice communication modalities as described herein, the session may be a voice and/or video call.

At 1209, all the transaction logs are synchronized to the customer's CRM in real time or in a batch process as previously described.

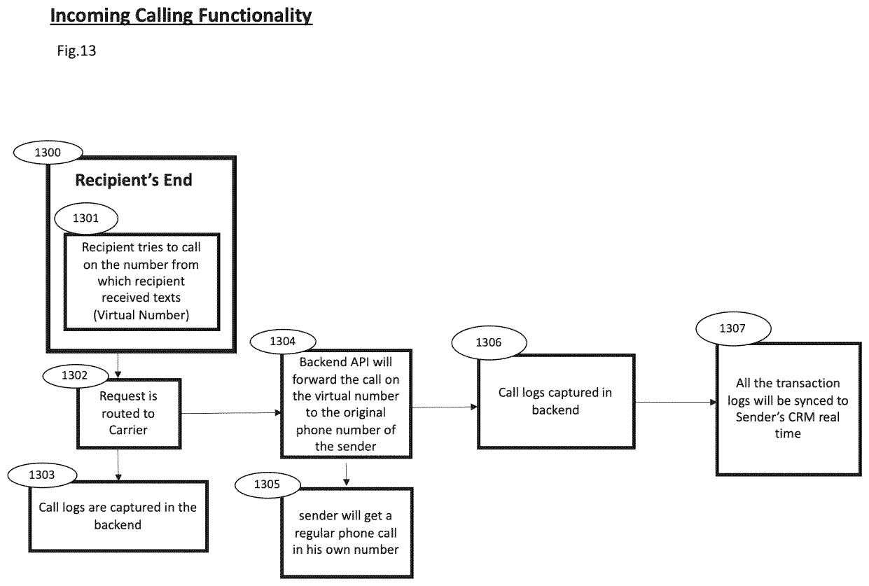

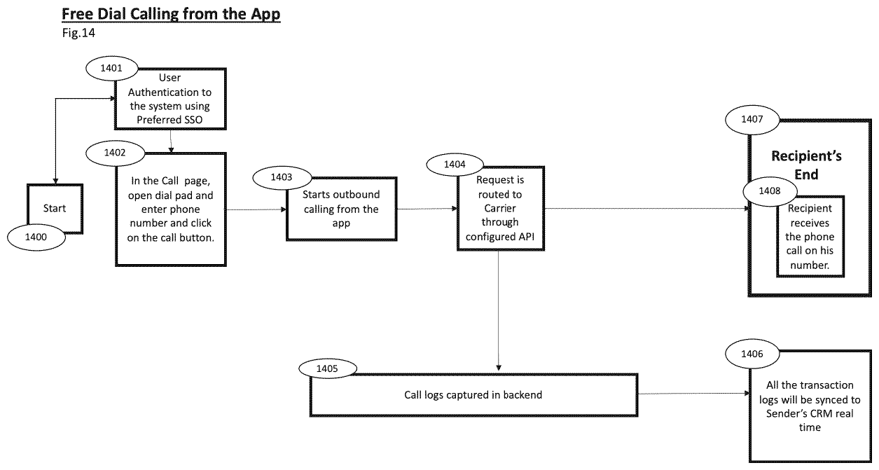

FIG. 13 shows a non-limiting, exemplary method for inbound communication from a patient to a healthcare professional. A similar method could be used for communication between an external support staff member and an HCP or an HCP staff member. In this example, the recipient, who is assumed to be a patient, attempts to initiate a synchronous voice communication session according to the previously received session initiation request. For example, at the recipient's side, the recipient may wish to initiate a telephone or other voice conversation, based on a previously received message, at 1300. At 1301, the recipient may attempt to call back the telephone number from the request, which may be an SMS message or other message service message. The telephone number from the request from the healthcare professional is a virtual number, such that preferably a direct telephone call is not performed.

Instead, at 1302, the request is routed to a carrier for a telephone call, as in this example the recipient dials the number as for any other type of telephone call. At 1303, the call is preferably logged, even for unsuccessful connection attempts as previously described.

At 1304, the Backend API preferably forwards the call from the virtual number to the original phone number of the user, which in this example is a healthcare professional. At 1305, the user receives a regular telephone call on their number. At 1306, the call may be logged again at the backend. At 1307, all the transaction logs are synchronized to the customer's CRM in real time or in a batch process as previously described.

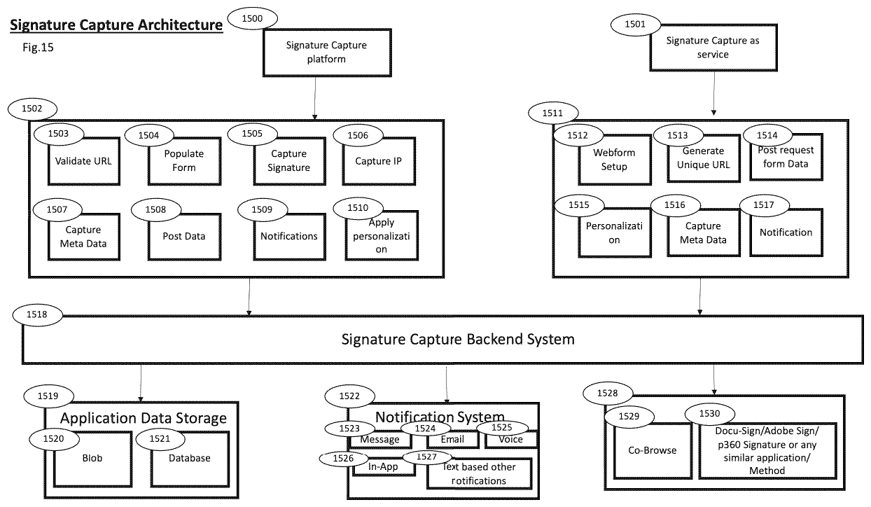

FIG. 14 relates to a non-limiting, exemplary method for calling a recipient who is not already present in the CRM. This method may be used for example for a new contact number. While the description relates to an outbound telephone call, other types of voice calls may also be performed. A similar method may be performed for sending a message to a contact that is not in the CRM for the first time as well.

As shown, the method begins at 1400 when the user decides to initiate such a call and activates the app or other interface to the system as described herein. At 1401, the user authenticates to the system using SSO or another login process as described herein. At 1402, the user navigates to the call page or screen. In the call page or screen, the user opens the dial pad and enters the phone number. The number may be entered by manipulating the on-screen GUI (graphical user interface) buttons for the app, by entering numbers through the keyboard of the device, and/or by copying and pasting the number onto the screen. The user then clicks on the call button.

At 1403, the app causes the outbound call to be initiated. At 1404, the call is routed through the correct carrier, including a cellular provider, a landline provider, any VOIP (voice over IP) or other voice call service.

At 1405, preferably the call logs are captured at the backend, including with regard to initiated calls that are not answered, as well as those in which a conversation occurs. At 1406, all the transaction logs are synchronized to the customer's CRM in real time or in a batch process as previously described.

At 1407, the recipient receives the synchronous voice communication session initiation request, such that a synchronous voice communication session may begin. For example, for a call to a phone number of the recipient, the synchronous voice communication session may be a telephone call. For other voice communication modalities as described herein, the session may be a voice and/or video call. The recipient may choose to connect at 1408.

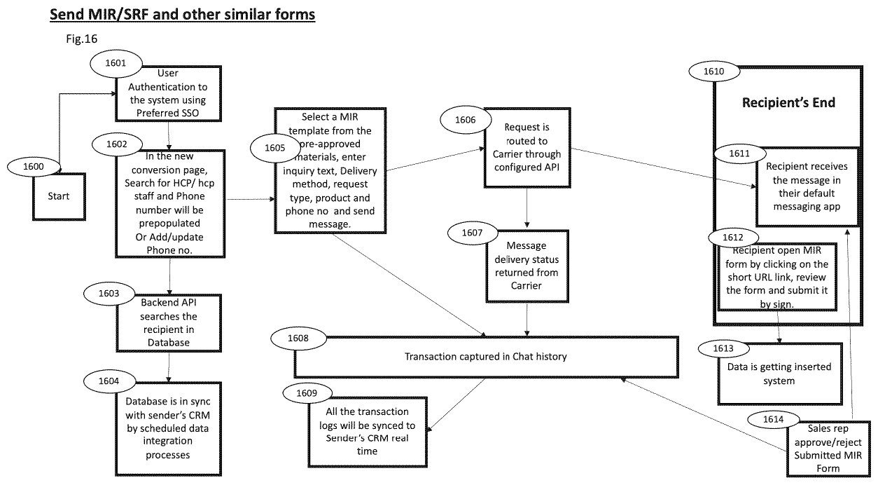

FIG. 15 shows a non-limiting, exemplary system for signature capture. A signature of a HCP or a HCP staff member may be required for authorization of a request, for example for medical information and/or a sample as described herein. As shown, a signature capture platform 1500 features a plurality of components 1502, including but not limited to a validate URL module 1503, which preferably validates the URL for which signature capture is requested. A populate form module 1504 may then populate the form which is to be signed and/or otherwise to provide the content for which assent or acknowledgement is agreed by the act of signing. A capture IP module 1505 preferably determines the IP address of the computational device which is being used to sign the form or other content, for example for security reasons and/or to be included with the signed form. A capture meta data module 1506 may also capture meta data relating to the computational device being used to sign the form or other content, and/or in relation to the time of day, date, day of the week and so forth. Such meta data may be captured for security reasons and/or to be included with the signed form. A post data module 1508 may provide this data and/or other information to a signature capture backend system 1518. Optionally, a notifications module 1509 notifies a requester of the signature, the signing entity or both, or other entities, upon signing of the form or other content. A personalization module 1510 optionally applies personalization to the form or other content, the act of signing and/or the notification.

Optionally, the signature may be captured through a signature capture service 1501. Such a service may be operated through a plurality of components 1511 as shown, including without limitation a web form setup module 1512, for entering the necessary information for the form. A unique URL generator 1513 may create a unique URL for this instance of the form at this time of signing. A post request form data module 1514, a personalization module 1515, a meta data capture module 1516 and a notification module 1517 may operate as previously described.

Signature capture backend system 1518 then preferably sends the transmitted information, which may include one or more of verification of the signature, the form information or other content, meta data, any security verification such as IP address verification, and so forth, to an application data storage 1519. Such information may then be stored in a blob form 1520 and/or a database 1521.

A notification system 1522 optionally receives notifications from the previously described notification modules, and then sends a notification through a suitable messaging modality, including but not limited to a message service 1523, an email service 1524, a voice service 1525, an in-app service 1526 and/or another type of text based notification 1527.

An API service 1528 may feature access to other services, including but not limited to co-browsing service 1529 and a further signature verification method 1530.

FIG. 16 relates to a non-limiting, exemplary method for sending a MIR template to a contact. The contact may for example be a HCP and/or a HCP staff member contact. Steps 1600-1604 may be performed as described with regard to steps 900-904 of FIG. 7. A MIR is a Medical Enquiry Request. Such a form may be used when particular medical, biological or other regulated information is requested, for example and without limitation with regard to side effects of a drug. Only certain personnel of a distribution and/or pharmaceutical manufacturing company are allowed by law to answer such a request for information. A similar process may be followed for a request to receive a physical sample, such as a SRF.

At 1605, the user selects a MIR template from the pre-approved materials. The user then enters a question and sends the SMS. The SMS or other message preferably includes a link to the MIR form, more preferably as a short URL as described herein.

Steps 1606-1609 may be performed as described with regard to steps 908-911.

Next, at the recipient side, at 1610, the recipient receives the message at 1611, preferably in their default messaging app. The type of default messaging app and the contact details for that app are preferably stored in the CRM, with regard to contact information for the recipient.

At 1612, the recipient opens the MIR form by clicking on the short URL link, reviews the form and submits it as signed. At 1613, the submitted information is received at the CRM and optionally at any other necessary database, such as a database required for regulatory compliance for example. At 1614, the sales representative, or other personnel of the company or organization who sent the message, approves or rejects the MIR submission. For example, the MIR submission may request information which the HCP or support staff is not entitled to receive, for example due to medical licensing requirements. The signature capture and sender approval process is to ensure the authenticity of HCP and HCP staff. A similar process may be performed for requesting a physical sample of a medical device, pharmaceutical or other medical supplies. These forms are preferably generated at run time.

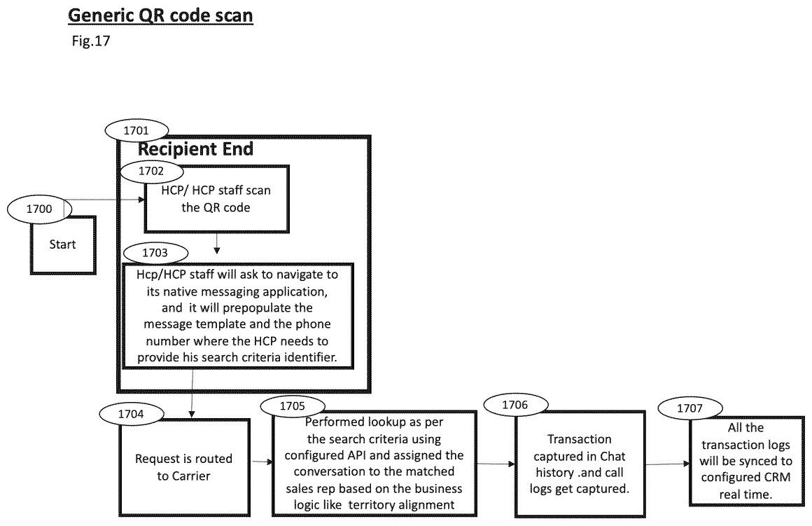

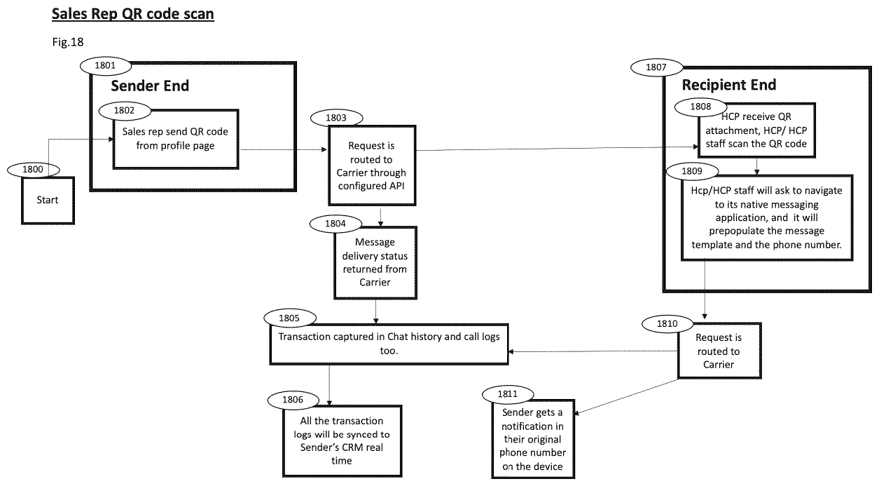

FIG. 17 relates to a non-limiting, exemplary method for scanning a code, such as a QR code, for initiating communication by a recipient. Once a QR code is scanned, the system identifies the HCP or HCP staff, for example according to location detection, zip code, territories, NPID inputs, and/or on the basis of inputs provided by scanner. The NPID is a unique identifier for HCP identification. This information is preferably matched against the conversations existing in the system to associate the contact. If the contact is not identified, it is preferably entered as an unknown number into the system for the sales representative or other personnel to check and follow up.

The process starts at 1700, when a recipient receives or has access to a QR code at 1701. For this non-limiting example, the recipient is assumed to be a HCP or HCP staff, although a similar process may be used for patients or other contacts.

At 1702, the recipient scans the QR code, for example with a smart phone or other internet-connected camera. At 1703, the recipient is asked to navigate to its native messaging application. The native application then prepopulates the SMS template, including with regard to the phone number. Such a template may require the recipient to provide a NPID or other identifier. The recipient may then send an SMS or other message with the required information.

At 1703, the message is routed to the correct carrier as described herein. At 1705, an NPID lookup is performed using the configured API. The conversation is assigned to the matched sales representative based on ZIP territory alignment or other information.

At 1706, preferably the message history is captured by the backend system as previously described. At 1707, all the transaction logs are synchronized to the customer's CRM in real time or in a batch process as previously described.

FIG. 18 shows an exemplary, non-limiting process for transmitting the previously described code, such as a QR code for example, to the recipient. The sender may be a sales representative or other representative of an external organization, who wishes to contact an HCP or HCP staff. The process begins at 1800, when the sender logs into the system as previously described. At 1801, the sender generates the code, such as the QR code for example. At 1802 the sender transmits the QR code from the profile page.

At 1803, the request is routed to the correct carrier for the type of message service that the message requires, as described herein. This routing request is preferably configured at the backend and is determined according to the contact details and/or preferences of the recipient.

At 1804, the message delivery status is optionally received from the carrier, if available. The message transaction is preferably captured in a chat history of communication between that authenticated user and the specific recipient at 1805. At 1806, all the transaction logs are synchronized to the customer's CRM in real time or in a batch process.

At 1807, the recipient receives the message in their default messaging app for that type of communication, such as an SMS message app on a mobile communication device for example. Preferably the default is set in advance and is stored in the previously described CRM. At 1808, the recipient receives the message with the code. The recipient then scans the code. At 1809, the contact is asked ask to navigate to its native messaging application, which then prepopulates the SMS template and the phone number as previously described.

This response request is then routed through the carrier at 1810. At 1811, the sender receives a notification of the response to the request, for example at their telephone number.

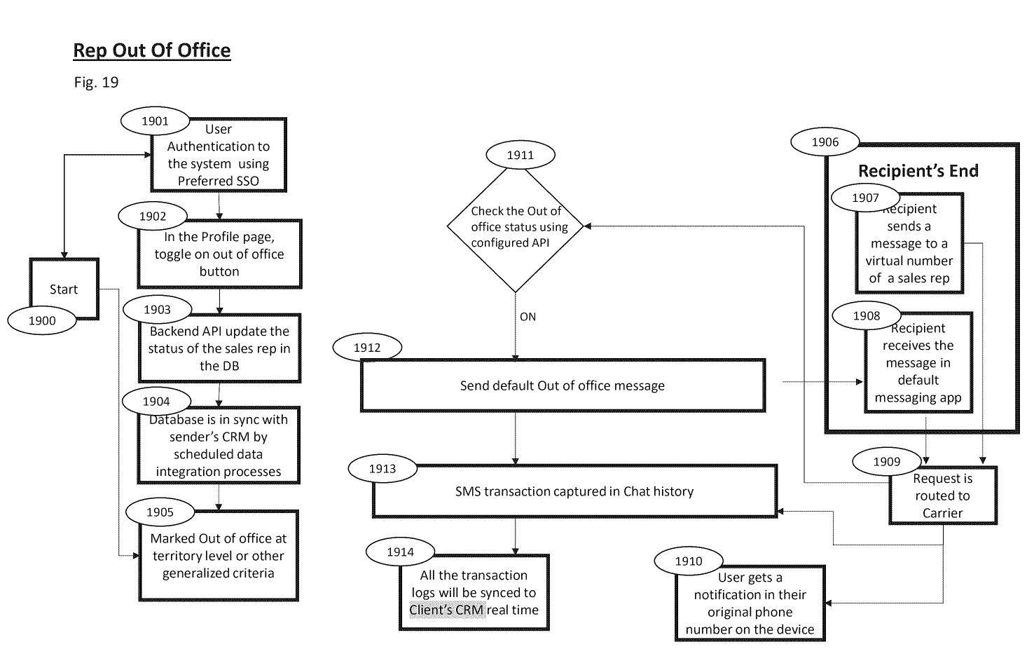

FIG. 19 shows an exemplary, non-limiting process for handling an out of office message. As shown, the user (who is the sender) may start the process by activating the system at 1900. The user is assumed for this example to be a sales representative or other representative of an external organization who wishes to contact a HCP or HCP staff, although other types of contacts may be included or substituted.

At 1901, the user performs user authentication to the system, for example using SSO (single sign on) technology as described herein. At 1902, optionally on the chat or profile page, the user toggles on the out of office function. Alternatively a calendar connection may be used to determine the status of the user automatically. At 1903, the backend API updates the status of the sales representative in the system database. At 1904, the database is synchronized to the backend in real time or in a batch process. At 1905, the user is optionally marked out of office at the territory level or according to other generalized criteria.

Turning now to the recipient's side 1906, at 1907 the recipient sends a message to the external representative contact, such as the sales representative. The message may be sent to a virtual number. At 1908 the recipient receives the message in their default messaging app. At 1909, the request is routed to the suitable carrier as described herein. At 1910 the recipient then receives a notification on their own device, for example as a message. At 1911, the system checks the availability status of the recipient through the backend API to the database. As the out of office status is activated, an out of office message is sent at 1912.

The message transaction is preferably captured in a chat history of communication between that authenticated user and the specific recipient at 1913. At 1914, all the transaction logs are synchronized to the customer's CRM in real time or in a batch process.

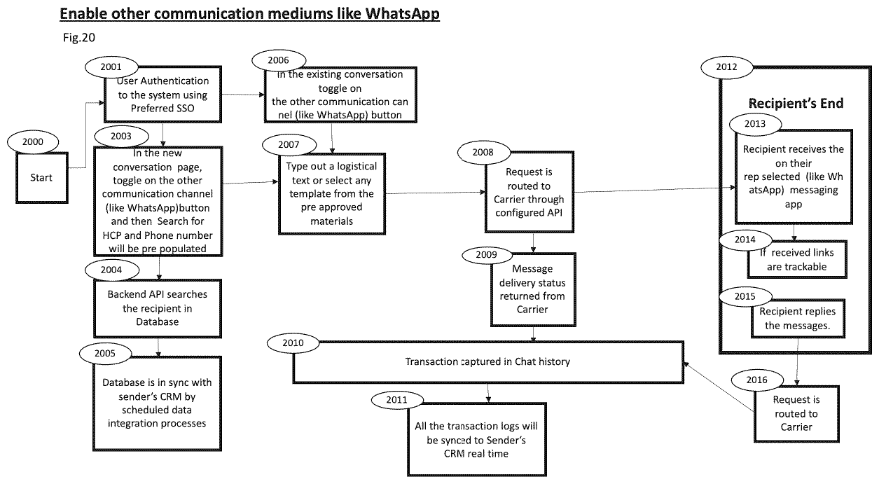

FIG. 20 shows a non-limiting, exemplary method for communication between an external support staff member and a recipient, who could be a healthcare professional (HCP) or HCP staff for example, using a specific example of a messaging service. This example for this method is the WhatsApp messaging service.

In this non-limiting example, the conversation is assumed to be between an HCP and an external support staff member. Preferably, this method is employed after communication is established between the healthcare professional and the external support staff member, whether generally or for this specific communication modality.

As shown, a healthcare provider, office staff member, external support staff member or other user initiates communication at 2000, for example by logging into a software interface. For the purpose of description only, the user is assumed to be an external support staff member attempting to contact an HCP (healthcare professional). The user in this non-limiting example is the sender of a message and the HCP is the recipient. The user is then invited to authenticate at 2001, for example by using an SSO (single sign-on) method, which may be performed through an existing email, social media or other user account. At 2006, the user (sender) may select an existing conversation to continue as described herein, after which the process continues at 2007

Alternatively, at 2003, the user may be shown a landing page and/or may employ a voice command to indicate which healthcare professional is to be contacted. If the phone number or other identifier of that healthcare professional is not available, then preferably the user enters it. Although the phone number is a preferred identifier for the healthcare professional, optionally a social media account identifier or other identifier may be used. Where suitable, such an identifier may be substituted for “phone number” when the latter is discussed herein. The user indicates that the messaging service to be used is WhatsApp. Alternatively, this messaging service selection may be made automatically according to the recipient's contact preferences.

At 2004, the backend API receives the request and searches for the correct healthcare provider information, for example in the previously described server database. The healthcare provider information may include for example contact details of the healthcare provider to be contacted, including but not limited to phone number and/or social media contact information. Optionally such contact information is for a “cut-out” communication modality, in which the private contact details of the HCP are kept private, while contact information that the server supports is provided. At 2005, the database is preferably synchronized with the CRM (customer relationship management) database of the external support staff company or organization by scheduled data integration processes. For example, such an synchronization may be performed through the client data integration API of FIG. 1 (not shown).

At 2007, the external support staff member determines the message structure and content, for example from one or more templates, and/or as free-form text, or a combination thereof. For example and without limitation if the healthcare professional has previously requested assistance with a particular good or service, and/or is requesting information, then a suitable template may be selected, for example to request that the healthcare professional call the external support staff member, login to a secure portal, or otherwise perform some action. Alternatively or additionally, the external support staff member may transmit information directly in the message. Optionally the external support staff member may request the HCP to perform some other action such as a booking an appointment. The text may also comprise logistical text as described herein.

At 2008, the request to send the message is routed as necessary to transmit it as previously described. In this non-limiting example, the message is a WhatsApp message and the request is routed to the WhatsApp service provider.

At 2009, the delivery status is returned from the message communication provider, which in this non-limiting example is the WhatsApp service, if available. The message transaction is preferably captured in a chat history of communication between that authenticated user and the specific recipient at 2010.

At 2011, all the transaction logs are synchronized to the customer's CRM in real time or in a batch process as previously described.

At 2013, the recipient receives the message in their default messaging app for that type of communication, which in this non-limiting example is the WhatsApp message service. In this non-limiting example, the recipient is assumed to be a healthcare professional. Again, preferably the default is set in advance and is stored in the previously described CRM. At 2014, any received links that have been sent through the message may be tracked; alternatively or additionally, any clicked links may be tracked.

At 2015 the recipient replies, whether because the message content requested a reply or independently. At 2016, the request is routed through the carrier for a WhatsApp message.

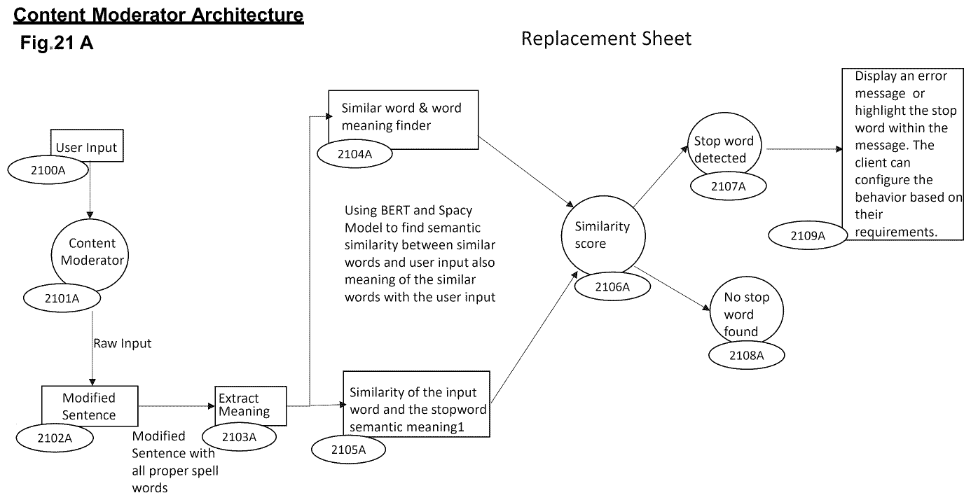

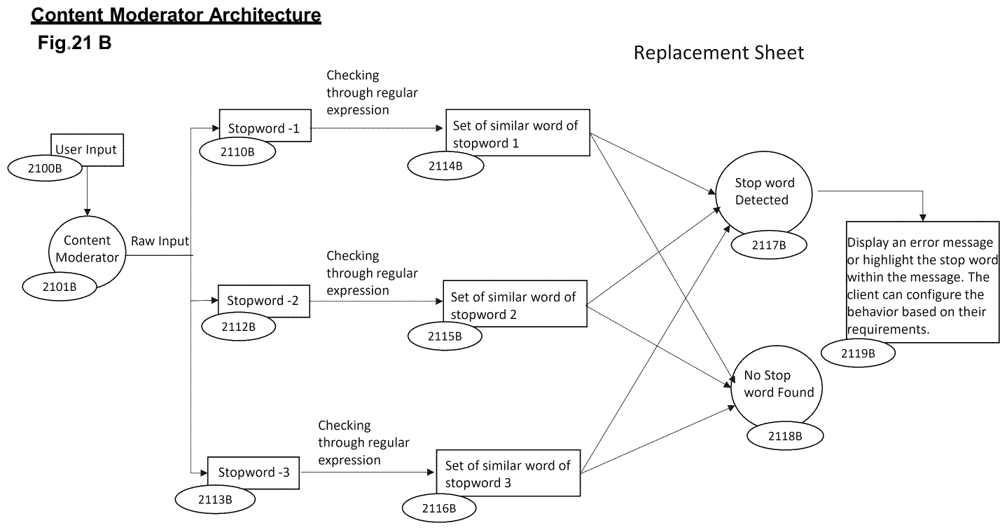

FIGS. 21A and 21B show a non-limiting, exemplary architecture for content moderation. The architecture receives user input at 2100, which is then routed to the content moderator module 2101. At the top branch, the raw input is preferably sent to a sentence modifier 2102, for example to check spelling of the words and/or grammar, and to modify or correct if necessary. A NLP (natural language processing) module preferably extracts meaning of the words and also of the sentences at 2103. A plurality of different processes may be applied to extract such meaning, shown as a wordnet 2104, to determine the meaning of individual words and/or their similarity to one or more stop words; and a sentence NLP analyzer 2105. Sentence NLP analyzer 2105 may apply various analysis techniques, including without limitation the BERT, ROBERTA and/or SENTENCEBERT techniques. Optionally the BERT and SpaCy models may be combined for analyzing the input text. These techniques may analyze word meaning in context and/or whole sentences or phrases. The output of both meaning analysis modules is then preferably fed to a similarity score module 2106, to determine similarity with one or more stop words, and/or other types of prohibited and/or controlled content. The latter may relate to phrases or sentences with permitted words but prohibited and/or controlled overall meaning. If the content requires further regulation, then the content may be passed to a further moderation process 2107. Alternatively, it is preferably passed to a process that does not require further moderation at 2108. Optionally, at 2109, an error message may be displayed if a stop word is detected, and/or the stop word may be highlighted within the user's input message.

The lower branch relates to a process in which a plurality of predetermined stop words are analyzed to find similar words, and both the original words and these similar words may be further compared to the raw content. This process preferably relies on word comparison. As a non-limiting example, three stop words, stop words 1, 2 and 3, are received through processes 2110, 2112 and 2113, respectively.

Next, stop words 1-3 are analyzed to find similar words, for example by checking through a dictionary and/or by using stemming or other techniques. These similar words are then compared to the raw content, for example by using regular expressions at processes 2114, 2115 or 2116 for stop words 1, 2 or 3, respectively. Any detected stop words preferably cause the content to be sent to a further moderation process 2117. For example, at 2119, an error message may be displayed if a stop word is detected, and/or the stop word may be highlighted within the user's input message. Alternatively, if no stop words are detected, then the content is preferably passed to a process that does not require further moderation at 2118.

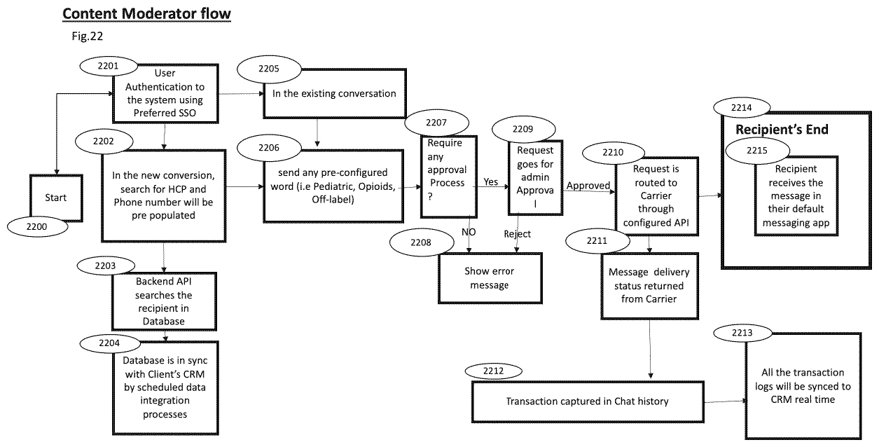

FIG. 22 relates to a non-limiting, exemplary method for content moderation of transmitted messages through the system as described herein. Steps 2200-2205 may be performed in a similar manner to steps 900-905 of FIG. 9.

At 2206, the user (sender) enters one or more words of the message, and/or selects a pre-created message, and/or edits a pre-created or suggested message. The suggested message may be generated by an AI model. The words are then analyzed for content moderation purposes, for example as described with regard to FIG. 21. For example, the content may be analyzed to detect one or more stop words, relating to restricted, controlled and/or prohibited content. The extent to which content requires moderation may be determined by organization policies. For example, for a distributor or manufacturer of pharmaceuticals, such content may relate to controlled substances, such as opioids for example; off-label use of a pharmaceutical (that is, a use that is not strictly allowed by a drug regulation agency); or pediatric uses.

At 2207, if the content does not require moderation, then it may be passed directly to 2210, for routing through the carrier as previously described. If the content does require moderation, but no moderation is available, then an error is raised at 2208 and the process preferably stops. If moderation is available, then at 2209, the content is reviewed. If allowed, then it is passed to the carrier at 2210.

From the carrier, the message delivery status is preferably returned at 2211. The message transaction is preferably captured in a chat history of communication between that authenticated user and the specific recipient at 2212.

At 2213, all the transaction logs are synchronized to the customer's CRM in real time or in a batch process as previously described.

At 2214, the recipient receives the message. The message is preferably received at 2215 in their default messaging app for that type of communication, which in this non-limiting example is the WhatsApp message service. In this non-limiting example, the recipient is assumed to be a healthcare professional. Again, preferably the default is set in advance and is stored in the previously described CRM. At 2014, any received links that have been sent through the message may be tracked; alternatively or additionally, any clicked links may be tracked.

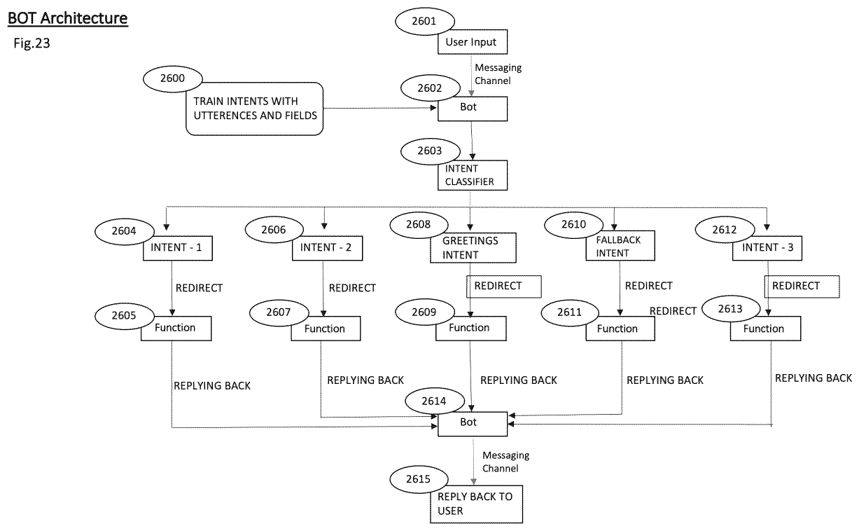

FIG. 23 relates to a non-limiting, exemplary architecture for bot communication. The architecture preferably trains the bot with a training module 2600. Training module 2600 preferably trains the bot on a combination of text words, sentences and phrases, and also previously determined intents. The intents may relate to a predicted intention of a sender of a message, including but not limited to, requesting information, registering a complaint, asking for a status update on a process, and/or information and/or a physical product that is expected to be received, and so forth.

The bot 2602 is trained by training module 2600, and then receives a user (or other sender) input 2601. Bot 2602 then classifies the intent of the input through an intent classifier 2603. Certain types of input may relate to a generic intent, including but not limited to a greetings intent 2608. If this intent is detected, then the input is preferably redirected to a further analysis function 2609, to see if any additional information is contained in the input. The intent plus any further information is then preferably passed to a response bot 2614. Response bot 2614 preferably is capable of NLG (natural language generation). Response bot 2614 then sends a response back to the user (sender) at 2615, according to the intent and the further information, if available.

Other intents may relate to specific types of communication, for example as previously described. These intents may be separately detected and are shown as intents 1, 2 and 3 for the purpose of illustration only and without any intention of being limiting. Intents 1-3 are received at 2604, 2606 or 2612 as shown, respectively. The analysis then performed as described with regard to further analysis function 2609 for the greeting intent, to see if any additional information is contained in the input, at functions 2605, 2607 or 2613, respectively. The process then continues as previously described.

If the input is not understood, or if no suitable intent analysis function is available, then the intent may be determined to be a fallback intent at 2610. Further information related to the fallback may be determined at function 2611. The process then continues as previously described.

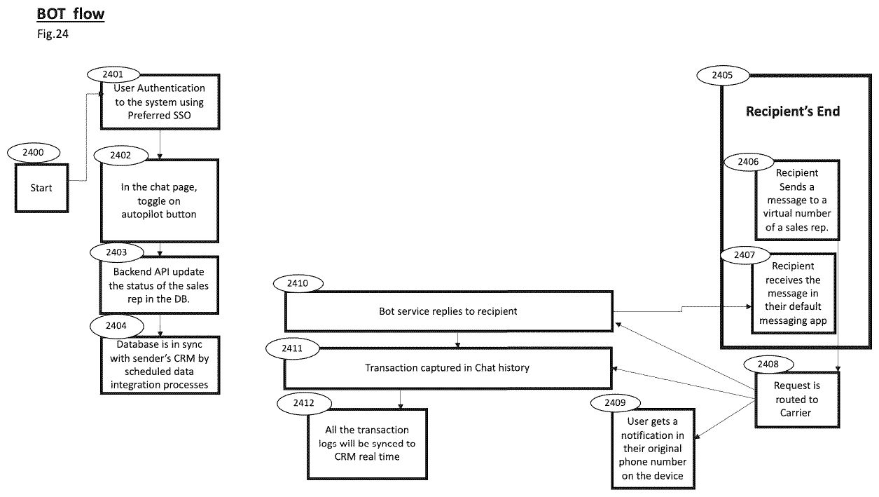

FIG. 24 relates to a non-limiting, exemplary method for bot communication. As shown, the user (who activates the bot) may start the process by activating the system at 2400. The user is assumed for this example to be a sales representative or other representative of an external organization who wishes to contact a HCP or HCP staff, although other types of contacts may be included or substituted.

At 2401, the user performs user authentication to the system, for example using SSO (single sign on) technology as described herein. At 2402, optionally on the chat page, the user toggles on the automatic reply (bot) function, so that the bot is able to respond in place of the user for example. Alternatively the bot function may be triggered automatically, for example when an out of office status is set. At 2403, the backend API updates the status of the sales representative in the system database, in terms of the activation of bot communication. At 2404, the database is synchronized to the backend in real time or in a batch process.

Turning now to the recipient side at 2405, the recipient may initiate a message communication session by sending a message at 2406. Alternatively, the message communication session may be initiated at the recipient side by receiving a message at 2407. In either case, message sending and receiving then continues as described herein. When the recipient sends a message at 2406, for example through their default messaging app, the message is preferably routed to the carrier at 2408 as previously described.

At 2409, the user may optionally receive a notification on their own device, for example as a message. At 2410, the bot service engages with the recipient to send and receive messages. The sent message is then routed back to the recipient, preferably at the default messaging app as previously described.

The message transaction is preferably captured in a chat history of communication between that authenticated user and the specific recipient at 2411. At 2412, all the transaction logs are synchronized to the customer's CRM in real time or in a batch process.

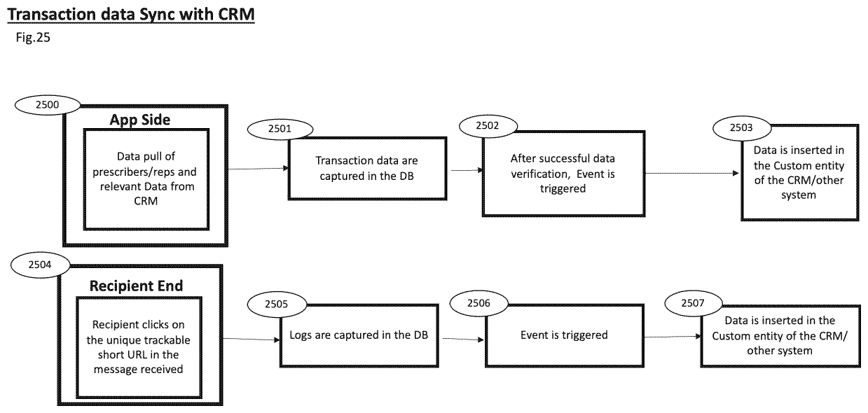

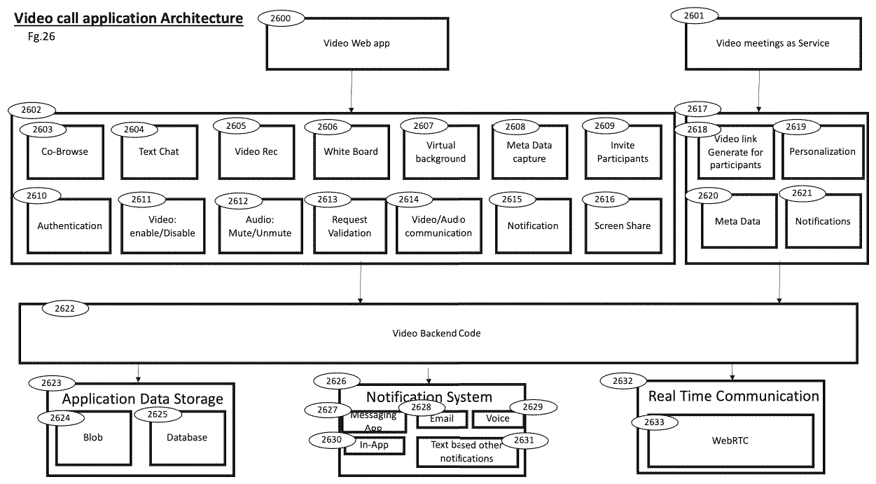

FIG. 25 shows a non-limiting, exemplary method for communication logging into a database. The method synchronizes information taken from communication records with the database. The database may for example be used by an external support staff member to coordinate customer information, in which the HCP and/or the HCP staff member may be a customer of the company employed by the external support staff member. Turning now to FIG. 25, at 2500, transaction information is captured from the message application of the HCP and/or HCP staff member, such that incoming/outgoing messages or calls from the application, and/or associated metadata, may be captured. At 2501, logs of such captured information are stored in the database, such as a CRM for example. At 2502, an event is triggered, for example according to the identity of the HCP and/or HCP staff member, and/or of the external support staff member. At 2503, data is inserted in the custom entity of the CRM, according to the triggered event, for example to update particular information.

On the recipient side, at 2504, with the recipient in this non-limiting example being the HCP and/or HCP staff member, the process preferably begins when the recipient clicks on the unique trackable short URL in the message received. At 2505, a log of such a click and any associated information, such as the message contents and/or the associated web page or micropage contents, are stored in the database, such as a CRM for example. At 2506, an event is triggered, for example according to the click of the URL and/or according to the identity of the HCP and/or HCP staff member, and/or of the external support staff member. At 2507, data is inserted in the custom entity of the CRM, according to the triggered event, for example to update particular information.

FIG. 26 relates to a non-limiting, exemplary architecture for a video call. The video call architecture may be supported through a video web app 2600. As shown, video web app 2600 features a plurality of components 2602, including but not limited to a co-browsing module 2603, which enables participants to view and optionally also interact with a web page together.

For co-browsing module 2603, as well as for other modules, optionally only one or some participants may use or activate such a module.

A text chat module 2604 may be present to enable participants in the video session to communicate by chat during the session. A video recording module 2605 may supporting capture of the data of the video session, including without limitation, recording audio and video, and optionally also capturing chat. A white board module 2606 may be provided to enable participants to draw on a white board during the video session. A virtual background module 2607 may enable video participants to change the background of their video capture.

A capture meta data module 2608 may also capture meta data relating to the computational device being used to view the video session, and/or in relation to the time of day, date, day of the week and so forth. Such meta data may be captured for security reasons and/or to be included with a recording of the video session.

An invitation module 2609 may enable participants to further invite additional participants to the video session, or to remind invited participants who have not yet joined to enter the video session.

An authentication module 2610 is preferably used to authenticate participants in the video session. Such authentication may be for limited participation, with limited access to one or more modules as described herein; or may permit additional access to such one or more modules. Preferably all participants engage with authentication module 2610 before entering the video session.

A video control module 2611 preferably enables each participant in the video session to determine whether to show their video. Alternatively, video from one or more participants may be blocked by video control module 2611, for example according to access privileges as described herein. An audio control module 2612 may perform a similar function for audio.

A request validation module 2613 may validate the format of a video session request, for example to determine whether the required parameters are available.

A communication module 2614 may support video and/or audio communication between the participants.

Optionally, a notifications module 2615 notifies a requester of the signature, the signing entity or both, or other entities, upon signing of the form or other content. Notifications and other data may be passed to a video backend 2622.

A screenshare module 2616 may enable one or more participants to share their computer screens with other participants.

The video call architecture may also be supported through a video meetings service 2601. Such a service may be operated through a plurality of components 2617 as shown, including without limitation an unique URL generator 2618, which may create a unique URL for this video session, whether for all participants or for a single participant (in which case, preferably each participant receives a unique URL). A personalization module 2619, a meta data capture module 2620 and a notification module 2621 may operate as previously described.

Video backend 2622 then preferably sends the transmitted information, which may include one or more of verification of the signature, the form information or other content, meta data, any security verification such as IP address verification, and so forth, to an application data storage 2623. Such information may then be stored in a blob form 2624 and/or a database 2625.

A notification system 2626 optionally receives notifications from the previously described notification modules, and then sends a notification through a suitable messaging modality, including but not limited to a message service 2627, an email service 2628, a voice service 2629, an in-app service 2630 and/or another type of text based notification 2631.

An real time communication service 2632 may feature access to other services, including but not limited to a WebRTC service 2633.

Optionally, the video call architecture may be provided as a video feature, for example as a widget, such that an option to start a video call may be embedded at different locations. For example, such a widget may be embedded on a web page or in another app.

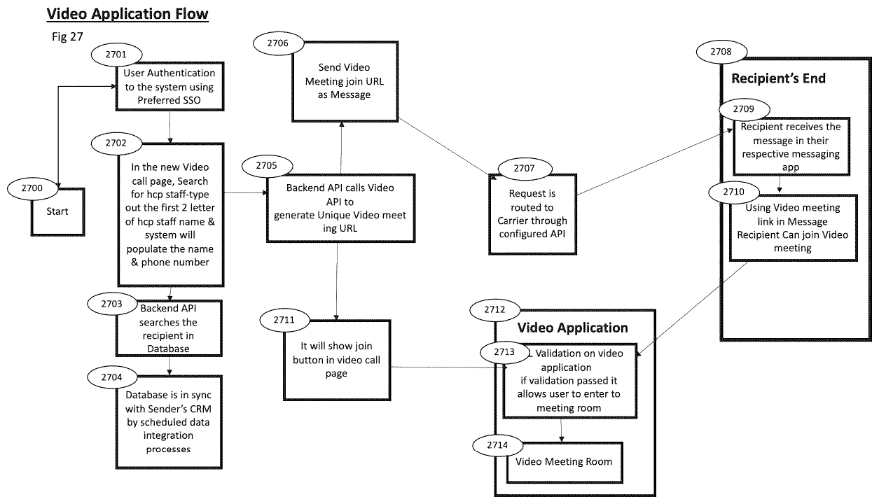

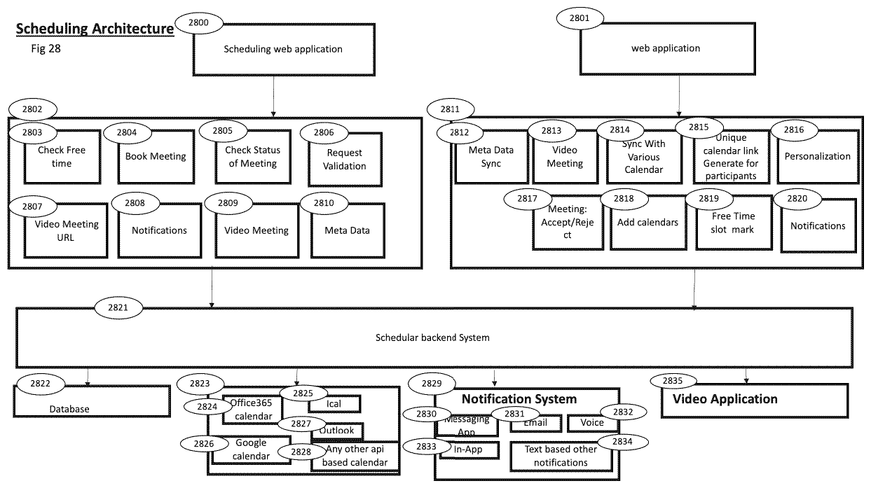

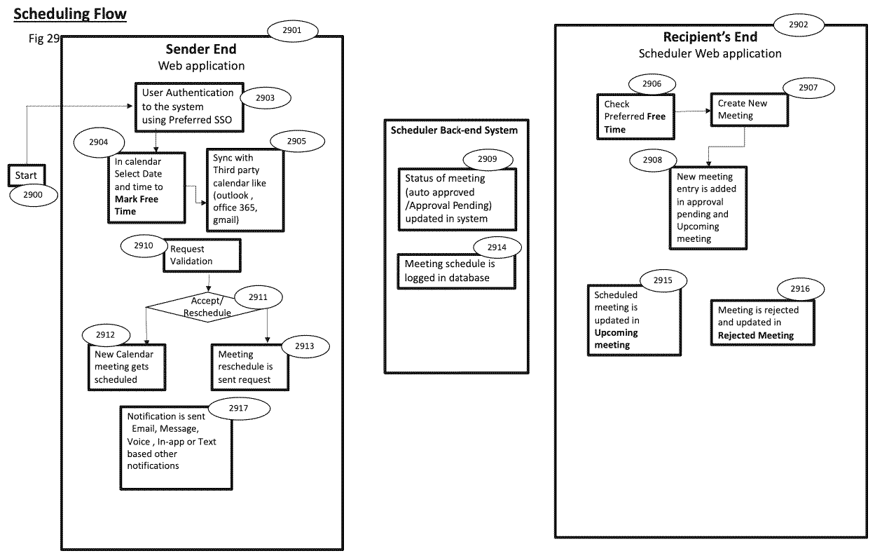

FIG. 27 relates to a non-limiting, exemplary method for a video call. As shown, stages 2700-2704 may be performed in a similar manner as stages 900-904 of FIG. 9. At 2705, the backend API preferably calls the video system API to generate at least one unique video session URL; optionally one such URL is generated per participant. At 2706, the video invitation URL is sent to at least one participant as a message. At 2707, the requested is routed through the carrier as previously described. At the recipient 2708, the recipient receives the message with the video link in their respective messaging modality at 2709. At 2710, the recipient joins the video meeting by clicking on the video link and/or by entering meeting information into a video meeting app or webapp.

Turning back to 2711, the backend preferably sends validation and/or authentication information, and optionally other details about the meeting (including for example permission(s) granted to the participants) to video application server 2712. At 2713, according to such validation information, server 2712 determines whether the recipient is permitted to enter the meeting room. If validated, then the recipient enters at 2714.

FIG. 28 relates to a non-limiting, exemplary architecture for scheduling a call. The scheduling architecture may be supported through a scheduling web app 2800. As shown, scheduling web app 2800 features a plurality of components 2802, including but not limited to an availability searching module 2803, which supports finding an available time for at least one and preferably a plurality of participants.

A meeting booking module 2804 enables the video meeting to be scheduled, either after receiving an available time for one or more participants, or according to a manual scheduled meeting. The status of the meeting may be checked at 2805, for example to determine whether one or more participants have indicated that they cannot attend the meeting. A validation module 2806 is preferably used to validate participants who are to be scheduled in the meeting. Such validation may be for limited participation, with limited access to one or more modules as described herein; or may permit additional access to such one or more modules.

A video meeting URL generator 2807 may create a unique URL for this video session, whether for all participants or for a single participant (in which case, preferably each participant receives a unique URL).

Optionally, a notifications module 2808 notifies such information as acceptance of a meeting, change in status for a participant, change in a meeting schedule and so forth. Notifications and other data may be passed to a scheduling backend 2821.

A video meeting module 2809 may support scheduling and/or setup of a video meeting as described herein. A capture meta data module 2810 may also capture meta data relating to the computational device being used to view the video session, and/or in relation to the time of day, date, day of the week and so forth. Such meta data may be captured for security reasons and/or to be included with a recording of the video session.

The scheduling architecture may also be supported through a scheduling service 2801. Such a service may be operated through a plurality of components 2811 as shown, including without limitation a meta data synchronization module 2812. Meta data synchronization module 2812 preferably collects meta data for the purpose of analytics, for example to determine who is scheduling a meeting and with whom, and also in regard to meeting completion rate, and so forth. A video meeting module 2813 may support creation of and/or the provision of a video meeting as described herein. A calendar synchronization module 2814 supports synchronization of a plurality of participant calendars, for example according to time zone and/or to send a meeting invitation. A calendar link generator 2815 optionally generates a unique calendar link for the scheduled meetings, optionally uniquely for each participant but preferably at least unique to the scheduled meeting. A personalization module 2816 may optionally personalize calendar choices, the preferred days and/or times for a meeting, and/or the invitation text, or other items. A meeting accept or reject module 2817 enables each participant to accept or reject a meeting, and optionally to request rescheduling.

A calendar addition module 2818 optionally enables a participant to share their calendar with the scheduling system, for example for automatic scheduling and/or to indicate preferred days and/or times for a meeting. A free time slot module 2819 may be used to positively indicate preferred and/or open meeting times for each participant. A notification module 2820 may notify such information as acceptance of a meeting, change in status for a participant, change in a meeting schedule and so forth. Notifications and other data may be passed to a scheduling backend 2821.

Scheduling backend 2821 preferably receives information about the scheduled meeting, the participants and optionally other details, and then preferably sends the transmitted information, which may include participant identification information, meta data, any security verification such as IP address verification or validation, and so forth, to a database 2822.

A calendar interface service 2823 optionally supports sharing and/or synchronization of, and adding of meetings to, one or more API based calendars as described herein. As shown, calendar interface service 2823 may include connections to calendars for Office 365 (2824); iCal (2825); Google calendar (2826); or any general API based calendar (2827).

A notification system 2829 optionally receives notifications from the previously described notification modules, and then sends a notification through a suitable messaging modality, including but not limited to a message service 2830, an email service 2831, a voice service 2832, an in-app service 2833 and/or another type of text based notification 2834.

A video meeting service 2835 may support video meetings as described herein, and may interact with the previously described scheduling web app 2800 and/or scheduling service 2801 through scheduling backend 2821.

FIG. 29 relates to a non-limiting, exemplary flow for scheduling a meeting as described herein, such as a video call. The flow is from a sender web application 2901 to a recipient web application 2902, through a process beginning at 2900. The flow is enabled by a scheduling backend, which may be implemented as described for example with regard to FIG. 28.

The flow is initiated at sender web application 2901, by user authentication to the system, for example using SSO (single sign on) technology, at 2903. The sender web application may then login to a calendar function to mark free and/or available time at 2904, and/or meeting preferences; or such information may be indicated separately. A specific requested meeting time may also be included. At 2905, the sender's calendar is preferably synchronized with the scheduling backend as previously described.

At 2906, at recipient web application 2902, preferred free time and/or preferred meeting times are checked. At 2907, a new meeting is created. At 2908, a meeting validation request is sent to the scheduling backend.

At 2909, the scheduling backend receives calendar information from sender web application 2901 and from recipient web application 2902. The status of the meeting is then updated in the system, such as for example pending approval, auto approved and so forth. At 2910, the sender web application 2901 requests validation to the scheduling backend. At 2911, the set meeting time is either accepted or rescheduled. If accepted, the meeting is then scheduled on the calendar at 2912. If the meeting time is to be rescheduled, then a reschedule request is sent at 2913 to the scheduling backend. The reschedule request is then logged at 2914 in the database of the scheduling backend.

At 2915, the scheduled meeting is updated, for example due to the previously described rescheduling request, and is accepted. Alternatively, the meeting time may be rejected at 2916.

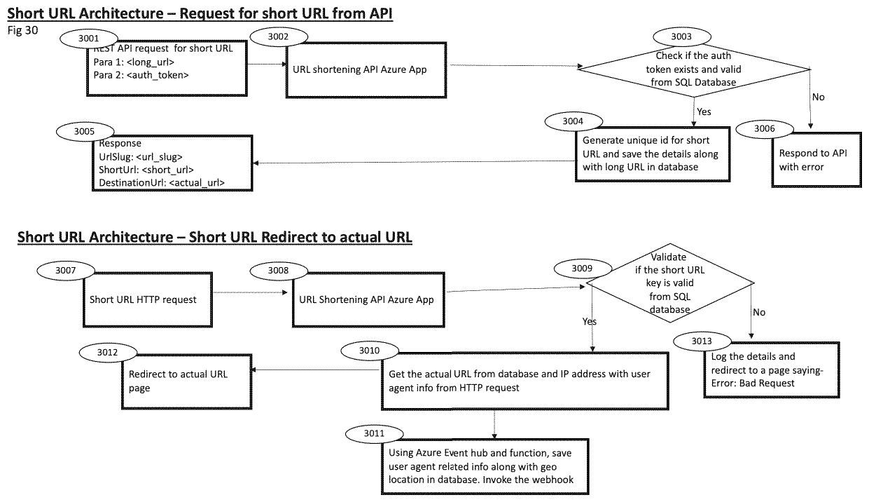

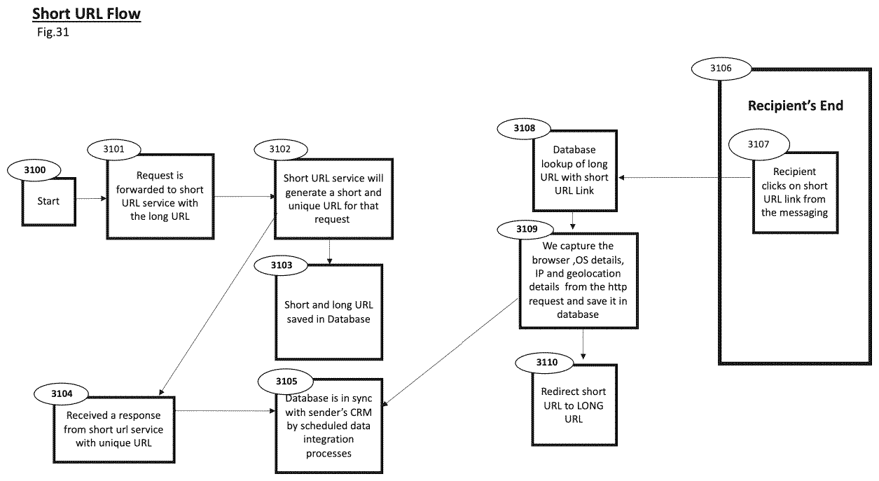

FIG. 30 relates to a non-limiting, exemplary architecture for short URL creation and redirection. The process of requesting the short URL starts at 3001, with a REST API request for a short URL. Preferably two parameters are passed: a long URL (that is to be shortened) and an authentication token. At 3002, these parameters are sent to a URL shortening app, such as the Azure URL shortening service. At 3003, the service determines whether the authentication token is valid. If so, then at 3004, a unique identifier and unique short URL are preferably created, and stored in the database, for correspondence to the long URL. At 3005, the response is sent, preferably with the short URL, and optionally with the long URL and a URL slug.

If the token is not valid, then at 3006, an error is sent in response to the API request.

The process of redirecting the short URL to the long (initial) URL starts at 3007 with a redirect request. At 3008, the application that created the short URL preferably receives it and starts the redirection process. At 3009, the short URL is validated, for example by checking against the database. At 30010 if validation succeeds, then the actual URL is retrieved from the database. Optionally, the IP address with the request information from the HTTP request is also stored, for example for security or logging purposes, at 3011. At 3012, the expanded URL is returned to the requester.

If the short URL is not validated, preferably an error message is returned at 3013.

FIG. 31 relates to a non-limiting, exemplary flow for short URL creation and redirection. As shown, the process starts at 3100, when a request is sent to the short URL service, including a long URL that is to be shortened, at 3101. At 3102, the short URL service generates a short and unique URL for that request, which is then stored in the database at 3103. At 3104, the unique short URL is sent in response to the request. At 3105, the database is synchronized with the CRM of the user, to include both the short URL and the original long URL.

At the recipient side 3103, the recipient receives the message. At 3107, the recipient clicks on the short URL link from the messaging. At 3108, the short URL redirects to the correct longer URL, through a database lookup. At 3109, various meta details are preferably obtained, including but not limited to the browser, operating system details, IP and geolocation details from the http request. These details are then saved in a database. At 3110, the redirect request itself is performed.

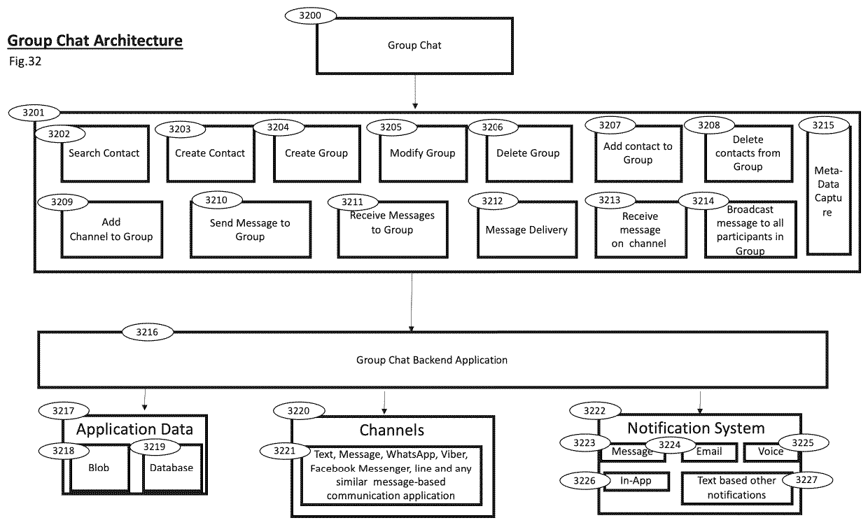

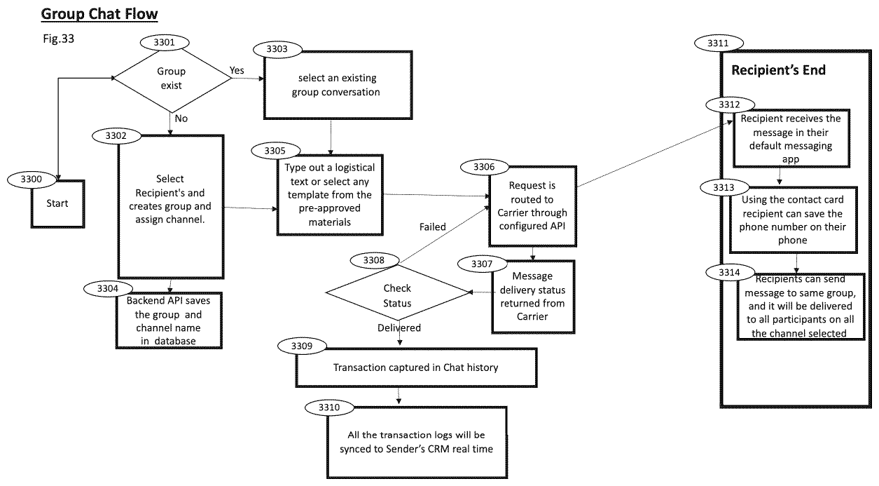

FIG. 32 relates to a non-limiting, exemplary architecture for a group chat. The group chat system 3200 preferably features a plurality of components 3201. These components may include but are not limited to searching a contact (3202), creating a contact (3203), creating a group (3204), modifying a group (3205), deleting a group (3206), adding a contact to a group (3207), deleting contacts from the group (3208), adding channel to the group (3209), sending a message to the group (3210), receiving a message by the group (3211), sorting message delivery (3212), receiving a message on channel (3213), broadcasting a message to all participants in a group (3214), and/or meta data capture (3215).

The group chat system 3200 then preferably communicates with a Group Chat Backend 3216. Group Chat Backend 3216 then preferably sends the transmitted information, which may include one or more of chat information, group addresses, meta data, any security verification such as IP address verification, and so forth, to an application data storage 3217. Such information may then be stored in a blob form 3218 and/or a database 3219.

Channels are preferably handled through a channels module 3220, which is preferably able to handle a plurality of channels, for example those selected from the group consisting of SMS messages, WhatsApp, Viber, Facebook Messenger, Line, any suitable similar message-based communication applications, and so forth.

A notification system 3222 optionally receives notifications from the previously described notification modules, and then sends a notification through a suitable messaging modality, including but not limited to a message service 3223, an email service 3224, a voice service 3225, an in-app service 3226 and/or another type of text based notification 3227. Notification system 3222 may for example send messages to the group chat through one or more different messaging systems. Optionally messages are routed through a plurality of messaging systems.

FIG. 33 relates to a non-limiting, exemplary flow for a group chat, between any suitable combination of a HCP, a staff member of an office of a healthcare professional, a patient or an external support staff member. In this non-limiting example, the communication is between a HCP and/or the staff member of the healthcare professional, and the external support staff member. The external support staff member initiates communication at 3300, for example by logging into a software interface, and then by authenticating, as previously described.

At 3301, the user determines whether the group exists. If not, then at 3302, the user selects the recipients (other group participants) and creates the channel. As noted below, a plurality of different messaging modalities may be used for the recipients, and/or a different messaging modality may be used for the user creating the group than for one or more other recipients. At 3304, the backend API saves the group details and channel name, or other identifier, in the database.

At 3303, if the group does exist then the existing group conversation is selected. At 3305, the external support staff member determines the message structure and content, for example from one or more templates, and/or as free-form text, or a combination thereof. For example and without limitation if the external support staff member wishes to transmit information or alternatively wants the HCP staff member to contact them, then a suitable template may be selected, for example to request that the HCP staff member call the external support staff member, the HCP and/or the office; login to a secure portal, or otherwise perform some action. Alternatively or additionally, the external support staff member may send information directly in the message.

At 3306, the request to send the message is routed as necessary to transmit it as previously described. As a non-limiting example, the message is an SMS message and the request is routed to a suitable carrier. As another non-limiting example, the message is transmitted through a different messaging modality, which may for example comprise a social media channel modality, in which case the request is routed to another suitable carrier.

At 3307, the delivery status is returned from the carrier or other message communication provider. At 3308, the delivery status is checked. If the message delivery status is failed, then preferably stages 3306 and 3307 are repeated. If the message delivery status is successful, then the message transaction is preferably captured in a chat history of communication between that authenticated user and the specific recipient at 3309. At 3310, all the transaction logs are synchronized to the customer's CRM in real time or in a batch process as previously described.

Turning now to the recipient's side at 3311, the message is received in the messaging modality app of the recipient at 3312, according to the modality used to send the message. The modality selected may be a default for the recipient, for example. Optionally, the message features a contact card. At 3313, the recipient may save the phone number, or other social media messaging contact details, or other messaging modality contact details. Such details may be saved only for the user (external staff member) and/or for one or more other participants in the group chat. The recipient may save such information on a mobile device, such that 3311 may represent the mobile communication device, including but not limited to a cellular telephone, a smart phone and the like.

At 3314, each recipient may then send a message to the same group included in the group chat, such that it is delivered to all participants on the selected messaging modality for each participant.

Optionally, multiple messaging modalities may be used for communication by different members in the group for the group chat. In such a situation, stages 3306-3308 are performed for each messaging modality separately, while the system tracks message transmission, content and receipt for compliance and auditing purposes.

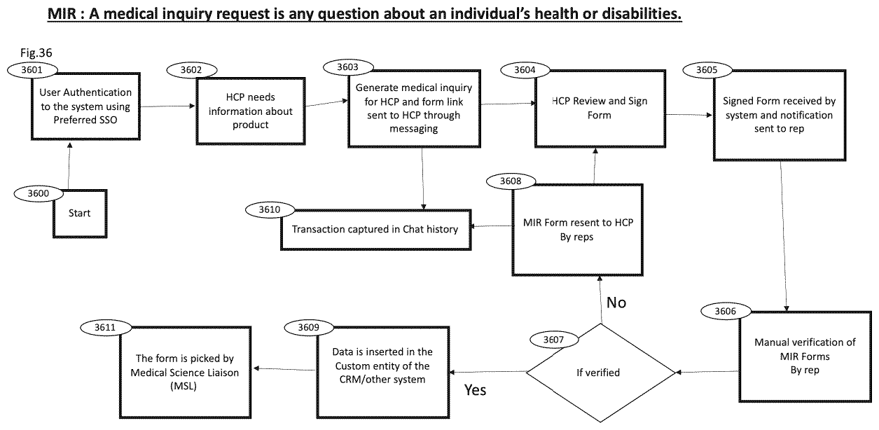

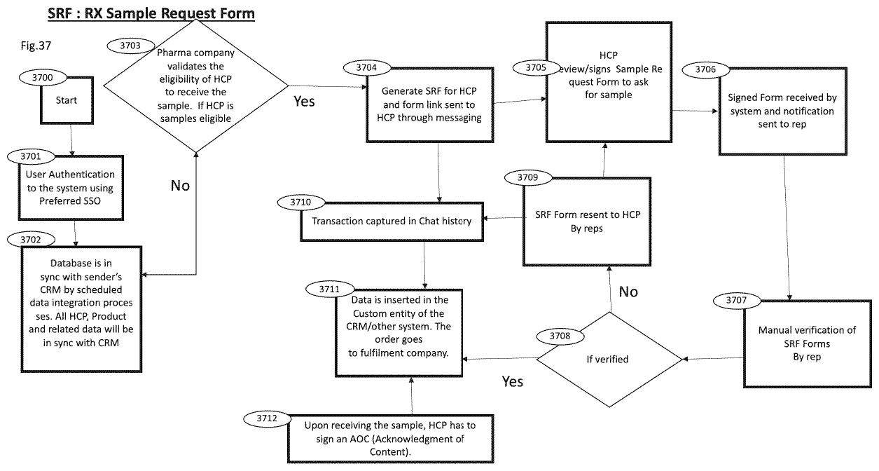

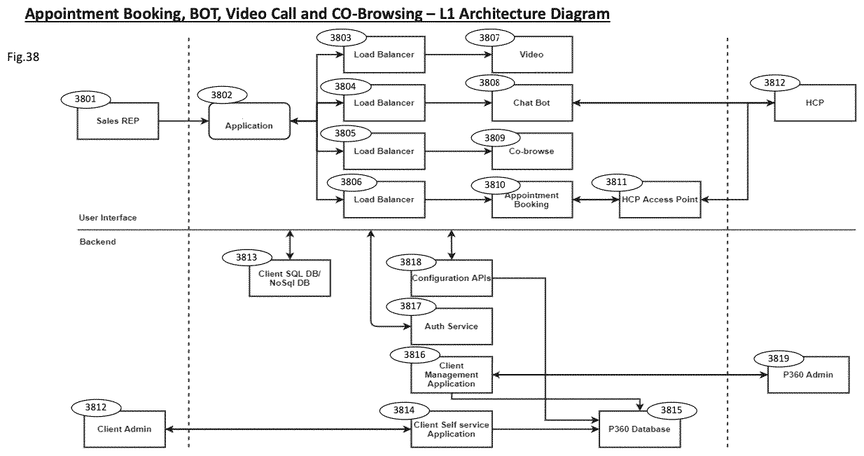

FIG. 36 relates to a non-limiting, exemplary method for handling a MIR request. The process begins at 3600, for example through a SSO login or other authentication process at 3601. At 3602, the user receives a request for information from a HCP contact. The request for information is in the form of a MIR request, which may be the result of the process described with regard to FIG. 16. Stages 3603-3605 may be performed according to the process as described with regard to FIG. 16.

At 3606, the filled in MIR request form is reviewed, whether manually by a sales representative or other agent, or through a NLP (natural language processing) algorithm. For example, the information in the MIR request form may be analyzed with regard to stop words and/or content that requires further regulation. At 3607, the result of the verification process may be reviewed to determine success or failure of verification. At 3608, if verification was not successful, the MIR form may be resent to the HCP. Otherwise, at 3609, if verification was successful, then data from the form may be inserted to a database and/or CRM, preferably associated with the HCP. The data may also be entered to a ticketing system, to be assigned to qualified personnel for reviewing the request. The data may then be pulled at 3611 by qualified personnel, such as for example a Medical Science Liaison (MSL). Also at 3610, each transaction in the system is preferably logged.

FIG. 37 relates to a non-limiting, exemplary method for handling a sample request, for a physical sample of a medical device, pharmaceutical, diagnostic kit or other medical product. The process starts at 3700, when the user authenticates to the system at 3701 as previously described. At 3702, the user reviews the database for the contact details of the HCP. Physical samples are expected to be more strictly regulated than information, such that preferably the process of fulfilling a request for a physical sample is not started unless the contact details of the HCP are already in the database and/or CRM.

At 3703, these contact details are sent to the company providing the sample, which is in this non-limiting example is a pharmaceutical company. The company preferably verifies that the HCP is eligible to receive the sample. For example, for some types of samples, licensing requirements may prohibit the company from sending the sample unless the HCP has that particular license. If the HCP is not eligible, then the process preferably stops.

At 3704, if the HCP is eligible, then a SRF (sample request form) is generated for the HCP and is sent with a signing link through a messaging modality. At 3705, the HCP reviews and signs the SRF as previously described with regard to the signing process herein. At 3706, the signed form is received and is sent to the sales representative or other agent of the company. At 3707, the SRF form may be reviewed as described in FIG. 36 with regard to the MIR form. At 3708, the verification result is reviewed, to determine whether it was successful or not.

If not successful, then at 3709, the SRF is generated and sent again to the HCP. The process then restarts. For example, the process may not be successful because the HCP has requested that the sample be sent to a medical office, clinic or other setting which does not fall under the license of the HCP or other licensing for that type of product. Such an additional level of control may be applied for controlled substances, such as opioid painkillers for example. Whether successful or not, all messaging transactions are preferably logged at 3710.

If the process at 3708 is successful, then at 3711, data regarding the SRF is preferably stored in a CRM or other database. A ticket may also be generated to enable fulfillment of the requested sample. Upon receiving the sample, the HCP signs an AOC (Acknowledgment of Content) at 3712, to acknowledge receipt of the sample. This acknowledgment is also preferably stored in the CRM or other database.

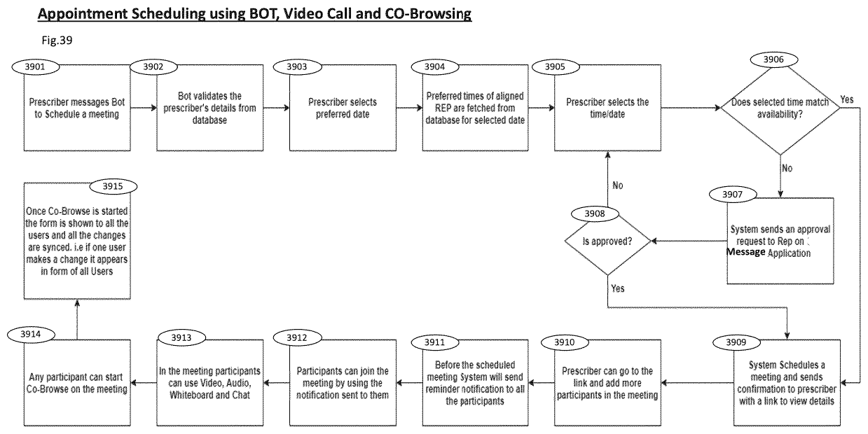

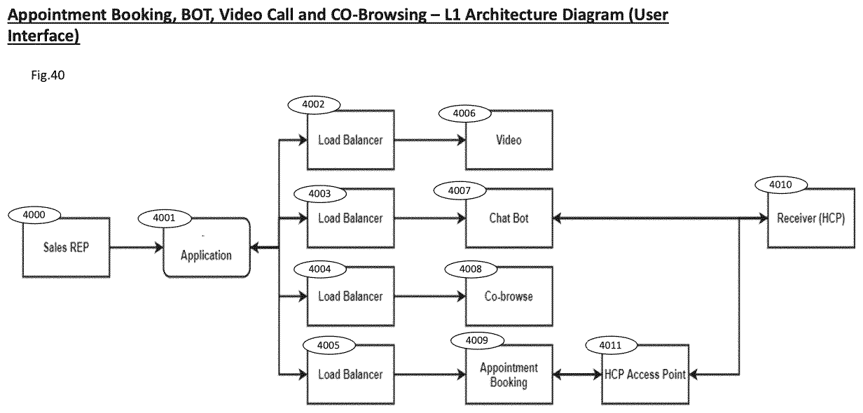

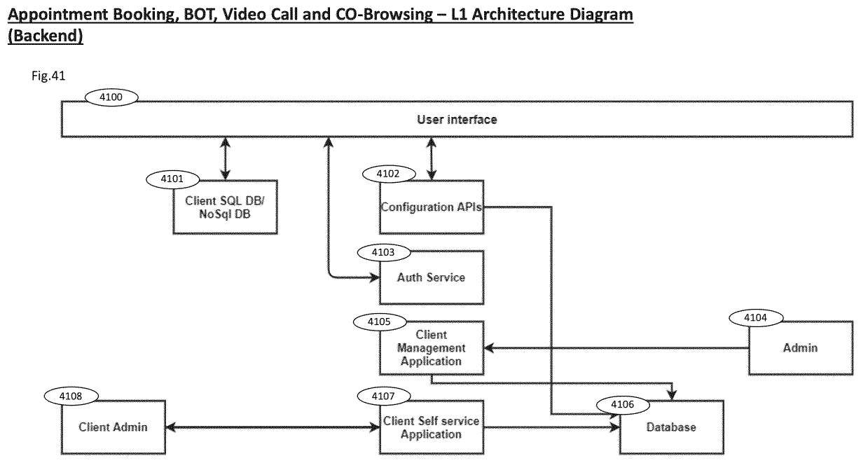

FIG. 38 shows a system for supporting communication with regard to multiple modalities, while FIG. 39 shows a non-limiting, exemplary method for communication through such a system. The operation of FIG. 38 is described with regard to FIGS. 40 and 41, which shows the system of FIG. 38 in more detail.

As shown, the process begins at 3901 when a HCP, described herein as a prescriber as a non-limiting example, messages a bot or other automatic communication software through a computational device and a messaging modality, to schedule a meeting. For example, the recipient may use SMS to message the bot. At 3902, the bot verifies the recipient's details through a database. Optionally only after verification is complete and a return message is received from the bot, or alternatively before such verification is complete, the recipient selects a preferred date at 3903. At 3904, the preferred times for a particular representative, medical professional, external support staff or another entity are shown. At 3905, the recipient selects the time and date for the appointment. At 3906, the system checks whether the selected time matches availability for the entity who will meet. If not, then at 3907, the system sends a message to that entity to see if in fact such a meeting is possible. The entity responds at 3908. If the meeting is not possible, then the process returns to 3905.

If a time and date are considered to be possible, either automatically through the system or alternatively through a manual approval process, then at 3909, the system schedules a meeting and sends the details to all participants, preferably including at least the recipient who initiated the meeting and the entity with whom the meeting was scheduled. For example, if the recipient who initiated the meeting is a medical professional, and the entity with whom the meeting was scheduled is a sales person or other representative of a company providing healthcare services and/or products, then preferably both the medical professional and the sales person or other representative receive the meeting details through the appropriate computational device. Optionally such meeting details include a link for the meeting and/or a calendar invite, and are sent through an appropriate message modality, which in this example may also include email.

At 3910, optionally the recipient is able to use the link to invite one or more additional participants to the meeting. At 3911, optionally a reminder is sent to all participants before the meeting is to start. At 3912, the participants are preferably able to join the meeting through the invitational link that was sent to them. At 3913, optionally multiple meeting modalities may be used, including but not limited to audio, video, whiteboard and/or chat. At 3914, optionally co-browsing is used to show online material to one or more meeting participants. Also optionally at 3915, co-browsing is shown to all participants and changes from one participant are synchronized to other meeting participants.

FIG. 40 shows a non-limiting, exemplary user interface system for communication through multiple modalities. In this system, a sender 4000, described as a sales representative in a non-limiting example, and a recipient 4010, described as a HCP as a non-limiting example, communicate with each other. Although the recipient is shown as the entity who is able to book an appointment, optionally the roles of sender and recipient may change, even for the same individual or entity, depending on the context. For example, in one situation, sender 4000 may be a healthcare professional, while recipient 4010 may be a patient, caregiver or an external support staff member for example. In another situation, sender 4000 may be a patient, caregiver or an external support staff member for example, while recipient 4010 may be a healthcare professional.

Sender 4000 may use a messaging modality 4001, which in this non-limiting example is SMS messages, to contact recipient 4010. Recipient 4010 may respond to such a contact or alternatively may choose to initiate contact. In either case, recipient 4010 may choose to initiate engagement through a chatbot 4007, for example to ask a question, request a chat conversation and/or to initiate an appointment. When the time comes for the appointment, or to book the appointment, recipient 4010 may initiate access through a recipient access point 4011, to reach an appointment booking module 4009. At the time of the appointment, sender 4000 and recipient 4010 may interact through a video module 4006 and/or a co-browsing module 4008. Load balancers 4002-4005 may help to support multi-modal communication between sender 4000 and recipient 4010.

FIG. 41 shows a non-limiting, exemplary backend system for communication through multiple modalities. As shown, a user interface 4100 preferably enables a user to access a client database 4101. In this non-limiting example, the user is an authorized user of an organization operating a system as described herein. For example, such an organization may comprise a HCP office or medical facility, and/or an external support staff organization. External support staff may include staff at diagnostic laboratories, other medical centers, and staff at providers of goods and services to healthcare professionals, including without limitation sales staff, pharmacists, medical device specialists, scientists and the like. Client database 4101 preferably stores information about the individuals and/or organizations served by the organization operating a system as described herein.

A configuration API 4102 may be used to configure a database 4106, which preferably stores information required for or in order to support a system as described herein. An authorization service 4103 may be used to determine authorized access to the backend system shown herein. Management of access to a particular aspect of the system as described herein may be controlled through authorization service 4103 and/or according to one or more policies determined through a client management application 4104 which may for example be determined by an admin application 4106. Required policy information may also be stored in database 4106.

A client admin application 4108 may contact a client self service application 4107, for example to adjust one or more parameters of the backend system, and optionally to store such adjustments in database 4106.

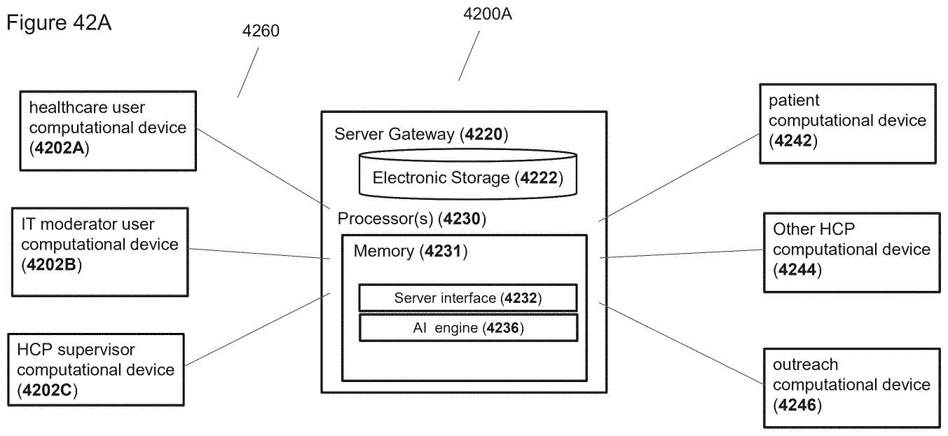

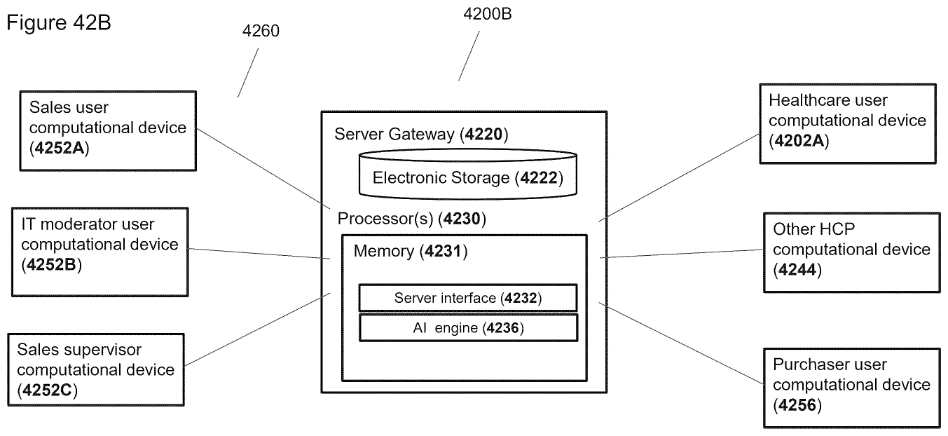

FIGS. 42A and 42B show exemplary systems for content moderation. As shown in FIG. 42A, a system 4200A features a plurality of participant computational devices 4202, shown as devices 4202A-4202C for the purpose of illustration only and without any intention of being limiting. Participant computational devices 4202A-4202C comprise a healthcare user computational device 4202A, an IT moderator user computational device 4202B and an HCP supervisor computational device 4202C. For the purpose of illustration only and without any intention of being limiting, in this example, healthcare user computational device 4202A creates content for transmission to another computational device, such as a patient computational device 4242, as described in greater detail below. Such content may comprise a text message, a video message, an audio message and so forth, sent through any suitable messaging service as described herein (not shown). Such content is preferably moderated for suitability, including but not limited to, compliance with HIPAA and other privacy regulations, avoidance of illegal content, avoidance of content that may not be proper for a particular recipient (such as a minor for example), and the like; as well as suitability in regard to guidelines made by the specific healthcare organization to which the healthcare user belongs and/or by professional and/or government regulators.

Determination of suitability may be made by IT moderator user computational device 4202B, whether manually or through an AI supported moderation service, such as an AI engine 4236, operated by a server gateway 4220. Participant computational devices 4202A-4202C communicate with server gateway 4220 through a computer network 4260. In this example, communication from healthcare user computational device 4202A may be sent through AI engine 4236. If deemed to be suitable, AI engine 4236 may send the communication through a suitable messaging service (not shown) to the recipient, such as patient computational device 4242. In case AI engine 4236 cannot determine whether such content is suitable, AI engine 4236 may send it to IT moderator user computational device 4202B, and/or to HCP supervisor computational device 4202C. IT moderator user computational device 4202B, and/or to HCP supervisor computational device 4202C may then label the content as to whether it is suitable; such labeling may be used to further train AI engine 4236.

Preferably AI engine 4236 also logs such content, for example at an electronic storage 4222 at server gateway 4220, or alternatively to an external database (not shown). The content log preferably includes the content of the message, the sender and accompanying details, and the recipient and accompanying details. Optionally details such as whether a supervisor reviewed the message, such as IT moderator user computational device 4202B, and/or to HCP supervisor computational device 4202C, is further included in the log. Such a message and accompanying details may be stored for a period of time, according to the regulatory requirements and/or the policy of the healthcare organization. Communication to and from server gateway 4220 may be performed through a server interface 4232 as shown.

To execute the functions of server gateway 4220, including without limitation the functions of AI engine 4236, server gateway 4220 preferably comprises a memory 4231 for storing a plurality of instructions and a processor 4230 for executing these instructions.

Recipient computational devices for receiving the previously described messages may include but are not limited to patient computational device 4242, another HCP computational device 4244, and an outreach computational device 4246, each of which may communicate with server gateway 4220 through computer network 4260, or, alternatively or additionally, may communicate with server gateway 4220 through a suitable messaging network as described herein.

For example, outreach computational device 4246 may be operated to reach out to patient communities and populations. Content that may be suitable for outreach computational device 4246 may not always be suitable for patient computational device 4242, and vice versa. Other HCP computational device 4244 may belong to the same healthcare organization as healthcare user computational device 4202A or a different healthcare organization; such a difference may in turn govern whether particular content is suitable for receipt by other HCP computational device 4244.

FIG. 42B also relates to content moderation, but in this non-limiting example, content is being sent from a sales organization, such as a pharmaceutical company, medical device company or other medical supplies company, to a healthcare organization. Components with the same numbering have the same or at least similar function as in FIG. 42A.

In a system 4200B, a plurality of sales organization computational devices 4252 are shown, as devices 4252A-C for the purpose of illustration only and without any intention of being limiting. Devices 4252A-C may comprise a sales user computational device 4252A, an IT moderator user computational device 4252B, and a sales supervisor computational device 4252C. Devices 4252A-C preferably communicate with server gateway 4220 through computer network 4260.

For the purpose of illustration only and without any intention of being limiting, in this example, sales user computational device 4252A creates content for transmission to another computational device, such as the previously described healthcare user computational device 4202A. Such content may comprise a text message, a video message, an audio message and so forth, sent through any suitable messaging service as described herein (not shown). Such content is preferably moderated for suitability, including but not limited to, compliance with HIPAA and other privacy regulations, avoidance of illegal content, avoidance of improper sales related content, and the like; as well as suitability in regard to guidelines made by the specific sales organization to which the sales user belongs and/or by professional and/or government regulators.

Determination of suitability may be made by IT moderator user computational device 4252B, whether manually or through the previously described AI supported moderation service, such as an AI engine 4236, operated by server gateway 4220. AI engine 4236 and server gateway 4220 may be different instances of the previously described components in FIG. 42A, trained to handle content moderation in relation to content being sent from a sales organization to a healthcare organization.

In this example, communication from sales user computational device 4252A may be sent through AI engine 4236. If deemed to be suitable, AI engine 4236 may send the communication through a suitable messaging service (not shown) to the recipient, such as healthcare user computational device 4202A. In case AI engine 4236 cannot determine whether such content is suitable, AI engine 4236 may send it to IT moderator user computational device 4252B, and/or to sales supervisor computational device 4252C. IT moderator user computational device 4252B, and/or sales supervisor computational device 4252C may then label the content as to whether it is suitable; such labeling may be used to further train AI engine 4236.

Preferably AI engine 4236 also logs such content, for example at an electronic storage 4222 at server gateway 4220, or alternatively to an external database (not shown). The content log preferably includes the content of the message, the sender and accompanying details, and the recipient and accompanying details. Optionally details such as whether a supervisor reviewed the message, such as IT moderator user computational device 4252B, and/or to sales supervisor computational device 4252C, is further included in the log. Such a message and accompanying details may be stored for a period of time, according to the regulatory requirements and/or the policy of the healthcare organization. Communication to and from server gateway 4220 may be performed through server interface 4232 as previously described.

Recipient computational devices for receiving the previously described messages may include but are not limited to healthcare user computational device 4202A, another HCP computational device 4244, and purchaser computational device 4256, each of which may communicate with server gateway 4220 through computer network 4260, or, alternatively or additionally, may communicate with server gateway 4220 through a suitable messaging network as described herein.

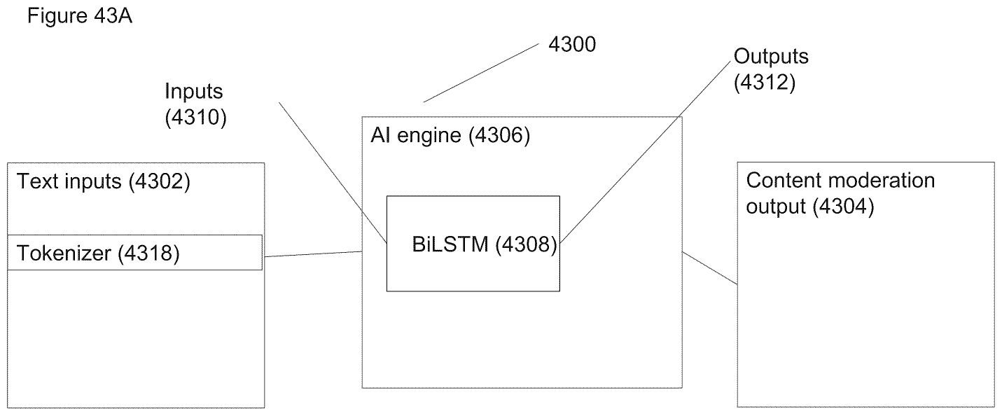

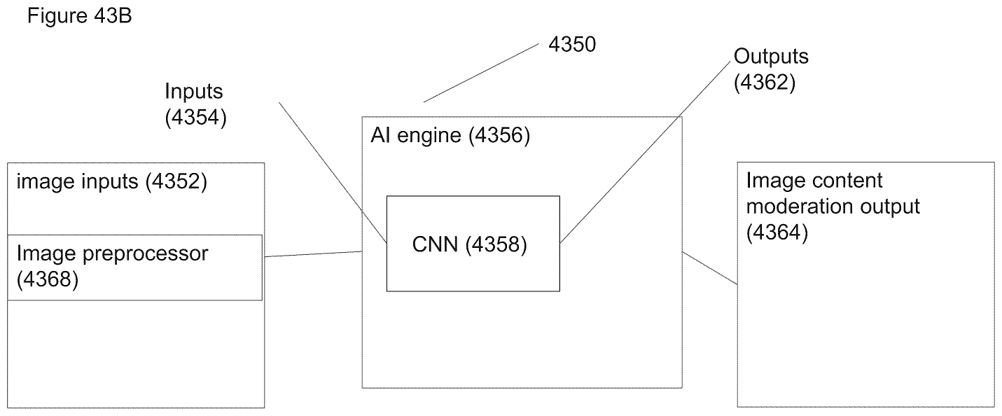

FIGS. 43A and 43B relate to exemplary systems for AI engines for content moderation. Turning now to FIG. 43A as shown in a system 4300, a user data interface 4302 provides text inputs that preferably are also analyzed with the tokenizer in 4318. A tokenizer is able to break down the text inputs into parts of speech. It is preferably also able to stem the words. For example, running and runs could both be stemmed to the word run. This tokenizer information is then fed into an AI engine 4306. Outputs 4312 are provided by AI engine 4306, resulting in a content moderation output 4304.

For example and without limitation, text inputs through user data interface 4302 may comprise the previously described content of a message, optionally and preferably along with accompanying information such as the identity of the sender and of the recipient, plus any characteristics of the recipient which may be relevant. Such content and accompanying information are then analyzed by AI engine 4306, to determine whether the content in the message is suitable as described herein. If the content may not be suitable, AI engine 4306 may reject the message and/or may transmit it to a human supervisor (through a computational device) for further review.

Optionally a plurality of AI models are used, to both capture (and analyze) user text inputs through NLP, and also to create a more accurate content moderation model, according to various factors that are modeled according to previous outcomes.

In this non-limiting example, AI engine 4306 comprises a BILS™ (bilateral long short term memory network) 4308. BILS™ 4308 features inputs 4310, processing through neural network and then outputs 4312. A BILS™ is an RNN layer that learns bidirectional long-term dependencies between time steps of time series or sequence data. These dependencies can be useful when you want the RNN to learn from the complete time series at each time step.

Inputs 4310 preferably receive the input data from a text inputs interface 4302, which may for example comprise tokenized language processed by a tokenizer 4304.

FIG. 43B relates to a non-limiting exemplary system 4350 with similar or the same components as FIG. 43A, except for the neural network model. In this case, the neural network comprises a convolutional neural network (CNN) 4358, with inputs 4354, and outputs 4362. CNN 4358 is a different model than that shown in FIG. 43A. CNN 4358 may be used for analyzing text inputs but in this non-limiting example is used to analyze image inputs 4352, which may be pre-processed by an image preprocessor 4356, before being fed to inputs 4354.

After analysis by CNN 4358 for content moderation, the resultant outputs 4362 are fed to an image content moderation output 4364. As previously described for the text outputs, inputs 4354 may comprise, for example and without limitation, the previously described visual content of a message and/or other visual content, optionally and preferably along with accompanying information such as the identity of the sender and of the recipient, plus any characteristics of the recipient which may be relevant. Such content and accompanying information are then analyzed by AI engine 4506, to determine whether the visual content is suitable as described herein. If the content may not be suitable, AI engine 4356 may reject the visual content and/or may transmit it to a human supervisor (through a computational device) for further review.

A CNN is a type of neural network that features additional separate convolutional layers for feature extraction, in addition to the neural network layers for classification/identification. Overall, the layers are organized in 3 dimensions: width, height and depth. Further, the neurons in one layer do not connect to all the neurons in the next layer but only to a small region of it. Lastly, the final output will be reduced to a single vector of probability scores, organized along the depth dimension. It is often used for audio and image data analysis, but has recently been also used for natural language processing (NLP; see for example Yin et al, Comparative Study of CNN and RNN for Natural Language Processing, arXiv: 14302.01923v1 [cs.CL] 43 February 20143).

Another non-limiting example of a suitable model that could be combined with, or used in place of, the CNN or DBN as described herein, is a sentence-transformer model, a sentence-transformer model. The term Sentence-Transformers stands for a group of models trained by the UKPLab and published under the sentence-transformers Python library. Those models have a number of advantages, including addressing the problem of ambiguity in creating vector representation of a single sentence. The BERT model uses the cross-encoder that requires passing a pair of sentences into the model to predict how similar they are. Some approaches of embeddings extraction for a single sentence include but are not limited to using the [CLS] final hidden state or applying the average pooling of the token-level embeddings received from the last hidden layer.

Sentence-Transformers propose their custom methodology of fine-tuning the BERT model in a Siamese and Triplet Network structure by applying a pooling layer to the output of the BERT last hidden layer and then calculating the custom objective. Datasets used to SentenceBERT model fine-tuning are the public datasets designated for NLI or STS tasks. More about the training strategy can be found in the Sentence-BERT: Sentence Embeddings using Siamese BERT-Networks article (Nils Reimers and Iryna Gurevych, ARXIV identifier 1908.10084, 243 August 2019).

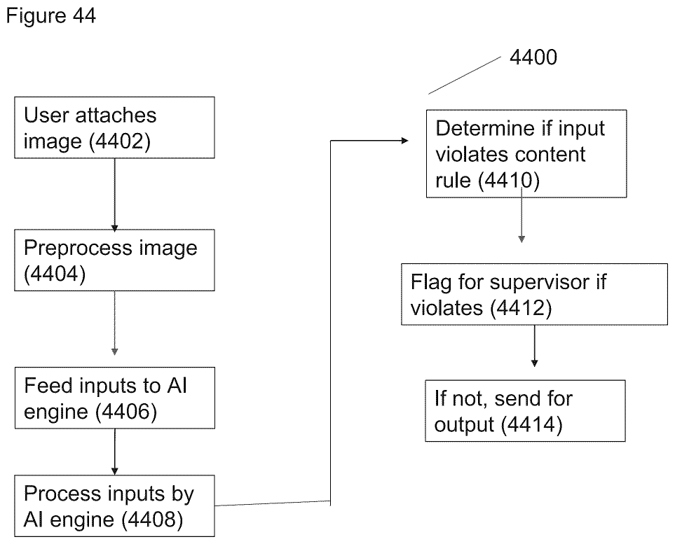

FIG. 44 relates to a non-limiting exemplary method for moderation of visual content. As shown in a method 4400, the method begins at 4402 when the user attaches an image to a message and/or attempts to transmit a message. At 4404, the image is preprocessed as necessary before AI analysis may be performed. At 4406, the inputs are fed to an AI engine. At 4408, the inputs are processed by the AI engine as described herein. At 4410, the AI engine as described herein determines whether the visual content violates one or more rules, also as described herein. It should be noted that these rules may be at least partially determined by the recipient of the visual content and/or message. For example, a transmission from one healthcare professional to another may not be suitable if made from a sales person to a healthcare professional, for example.

If the visual content is deemed to not be suitable, for example because it violates one or more rules, then at 4412, the AI engine may transmit the visual content to a supervisor for review as previously described. Otherwise, at 4414, the AI engine may allow the visual content to be transmitted.

A similar method may be performed for moderation of text content and/or messages, with the method being adjusted as necessary to accommodate text review and moderation.

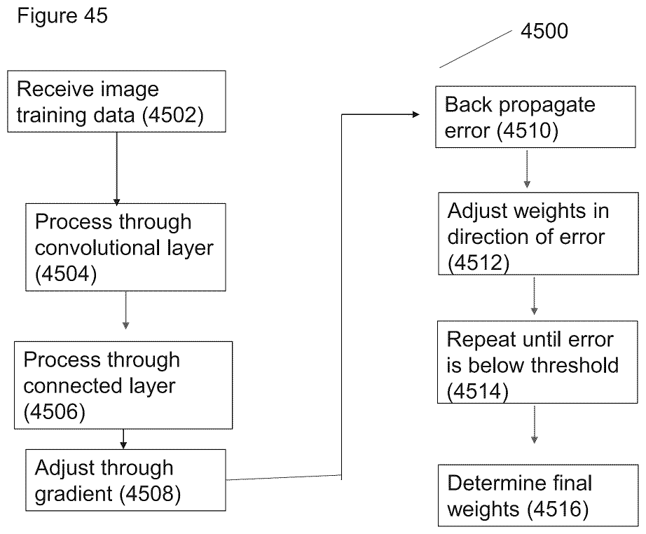

FIG. 45 relates to a non-limiting exemplary method for training an AI model for moderation of visual content. A similar method may be used to train an AI model for moderation of text content as is known in the art. The described method is suitable for training a CNN but may be adjusted for training other types of models as is known in the art.

A method 4500 begins at 4502, when image training data is received. The image training data may be labeled to indicate whether it is suitable or not. Optionally a plurality of different models may be trained on different types of images and/or to recognize images that violate one or more different rules. At 4504, the data is processed through the convolutional layer. At 4506, it is processed through the connected layer. Adjustments are made through the gradient at 4508. The error is calculated, in regard to whether the model is able to correctly process the labeled data to provide the desired outcome. This error may then be backpropagated through the model at 4510. The weights may be adjusted in the direction of the error at 4512. Other types of training methods are known; for example, two forward algorithms may be used in place of, or in addition to, back propagation. One of the forward algorithms may comprise positive labeled data; that is, data that the model should correctly analyze. The other forward algorithm may comprise negative labeled data, which indicates to the model what not to do.

At 4514, the process of at least 4504-4512 is preferably repeated until the error is below a desired threshold. At 4516, the final weights are determined.

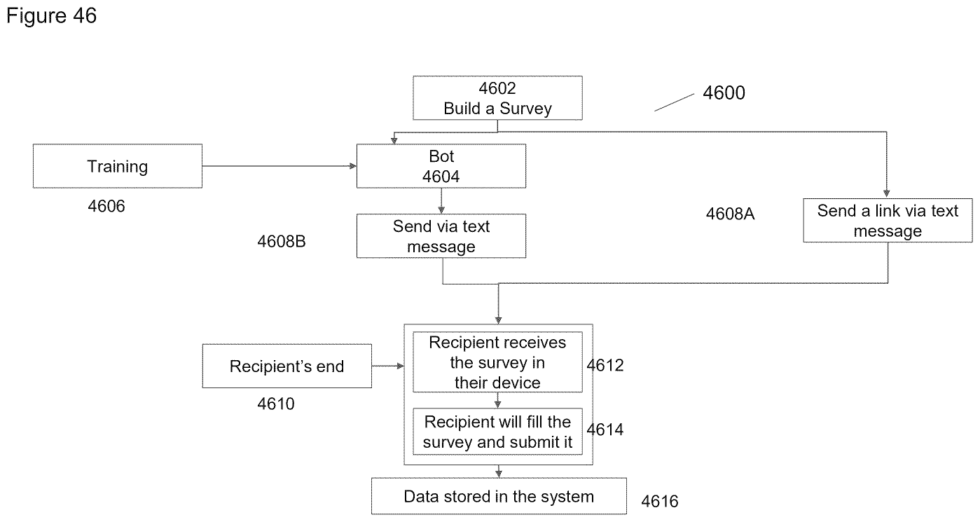

FIG. 46 relates to a non-limiting exemplary method for sending a survey. As shown in a method 4600, the method begins by initiating the process to build a survey at 4602, optionally by a previously described user through a previously described computational device. This process may proceed by sending the request to a bot 4604, which may comprise any suitable rules based and/or AI model. For the latter, preferably a training process 4606 is applied first, for example as previously described. The bot may construct the survey, which is preferably reviewed for suitability first through a previously described content moderation process (not shown). The survey may then be sent via text message at 4608B by the bot. The bot may also send a link through a text message at 4608A. Optionally, a user may manually construct a survey and send it by either mechanism.

At 4610, the recipient receives the survey, preferably through a personal device such as a mobile telephone for example at 4612. At 4614, the recipient fills in the survey and returns it. At 4616, the data is stored in the system.

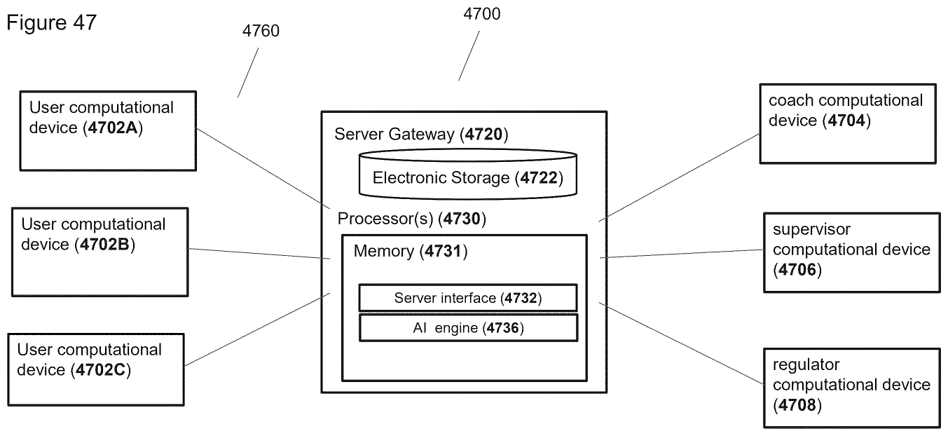

FIG. 47 shows an exemplary system for coaching. As shown in FIG. 47, a system 4700A features a plurality of participant computational devices 4702, shown as user computational devices 4702A-4702C for the purpose of illustration only and without any intention of being limiting. Participant computational devices 4702A-4702C communicate with server gateway 4720 through a computer network 4760.

Each user computational device 4702A-C may potentially receive coaching, for the purpose of illustration only and without any intention of being limiting, a coaching computational device 4704 may be used to provide human coaching. However, additionally or alternatively, such coaching may be provided through an AI engine 4736, operated by server gateway 4720. For example, user computational device 4702A may send one or more text and/or visual content messages. AI engine 4736 may analyze such communications and may determine that coaching is required, for example due to unclear wording, improper tone, non-suitable communication content and for any other reason. AI engine 4736 may then notify coaching computational device 4704 of the need for coaching, and/or may provide such coaching, to the user through user computational device 4702A.

The content of such coaching, whether provided by AI engine 4736, coaching computational device 4704 or both may be monitored by a supervisor computational device 4706. AI engine 4736 may also notify supervisor computational device 4706 of the need for coaching. Optionally supervisor computational device 4706 makes the final determination as to whether coaching is required. To assist in maintaining regulatory standards for communication, optionally a regulator computational device 4708 monitors such coaching.

Preferably AI engine 4736 also logs content which may indicate a need for coaching, and optionally also logs the coaching itself, for example at an electronic storage 4722 at server gateway 4720, or alternatively to an external database (not shown). The content log preferably includes the content of the message, the sender and accompanying details, and the recipient and accompanying details. Optionally details such as supervisor computational device 4706 and/or regulator computational device 4708, is further included in the log. Such a message and accompanying details may be stored for a period of time, according to the regulatory requirements and/or the policy of the healthcare organization. Communication to and from server gateway 4720 may be performed through a server interface 4732 as shown.

To execute the functions of server gateway 4720, including without limitation the functions of AI engine 4736, server gateway 4720 preferably comprises a memory 4731 for storing a plurality of instructions and a processor 4730 for executing these instructions.

Coaching as provided by AI engine 4736 may comprise suggesting alternative word choices for written and/or verbal content, rewriting the content for clarity, suggesting different and/or additional images and/or video content, adjusting the tone of the content, and so forth.

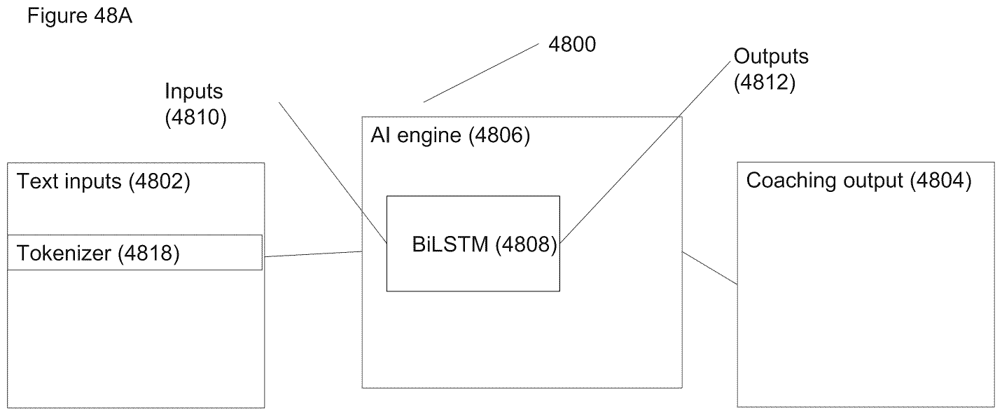

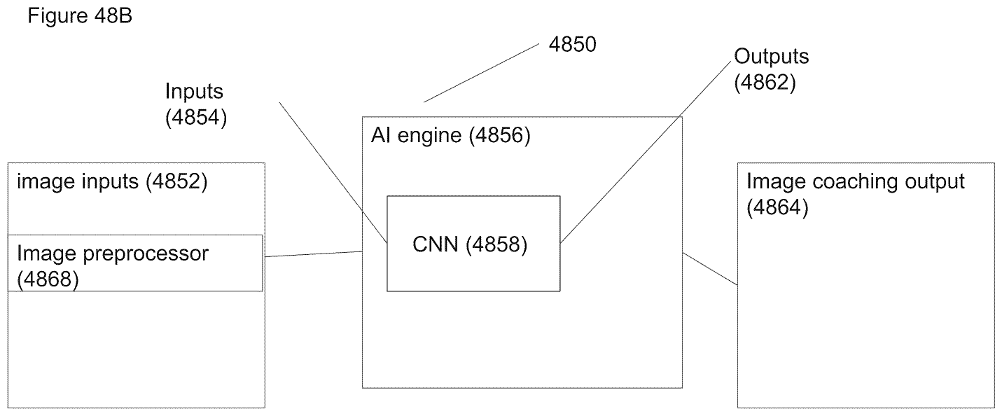

FIGS. 48A and 48B relate to exemplary systems for AI engines for coaching. Turning now to FIG. 48A as shown in a system 4800, a user data interface 4802 provides text inputs that preferably are also analyzed with the tokenizer in 4818. A tokenizer is able to break down the text inputs into parts of speech. It is preferably also able to stem the words. For example, running and runs could both be stemmed to the word run. This tokenizer information is then fed into an AI engine 4806. Outputs 4812 are provided by AI engine 4806, resulting in a coaching output 4804.

For example and without limitation, text inputs through user data interface 4802 may comprise the previously described content of a message, optionally and preferably along with accompanying information such as the identity of the sender and of the recipient, plus any characteristics of the recipient which may be relevant. Such content and accompanying information are then analyzed by AI engine 4806, to determine whether the content in the message is suitable as described herein and/or whether coaching is required. If the content may not be suitable and/or if coaching is required, AI engine 4806 may determine that coaching is required. Such coaching may be provided through AI engine 4806 as previously described and/or by a human through a computational device (not shown). Such coaching may comprise suggesting alternative word choices for written and/or verbal content, rewriting the content for clarity, suggesting different and/or additional images and/or video content, adjusting the tone of the content, and so forth.

Optionally a plurality of AI models are used, to both capture (and analyze) user text inputs through NLP, and also to create a more accurate content model for determining whether coaching is needed, according to various factors that are modeled according to previous outcomes.

In this non-limiting example, AI engine 4806 comprises a BiLS™ (bilateral long short term memory network) 4808. BILS™ 4808 features inputs 4810, processing through neural network and then outputs 4812. A BILS™ is an RNN layer that learns bidirectional long-term dependencies between time steps of time series or sequence data. These dependencies can be useful when you want the RNN to learn from the complete time series at each time step.

Inputs 4810 preferably receive the input data from a text inputs interface 4802, which may for example comprise tokenized language processed by a tokenizer 4804.

FIG. 48B relates to a non-limiting exemplary system 4850 with similar or the same components as FIG. 48A, except for the neural network model. In this case, the neural network comprises a convolutional neural network (CNN) 4858, with inputs 4854, and outputs 4862. CNN 4858 is a different model than that shown in FIG. 48A. CNN 4858 may be used for analyzing text inputs but in this non-limiting example is used to analyze image inputs 4852, which may be pre-processed by an image preprocessor 4856, before being fed to inputs 4854.

After analysis by CNN 4858 for coaching, the resultant outputs 4862 are fed to an image coaching output 4864. As previously described for the text outputs, inputs 4854 may comprise, for example and without limitation, the previously described visual content of a message and/or other visual content, optionally and preferably along with accompanying information such as the identity of the sender and of the recipient, plus any characteristics of the recipient which may be relevant. Such content and accompanying information are then analyzed by AI engine 4856, to determine whether the visual content is suitable as described herein. If the content may not be suitable, AI engine 4856 may reject the visual content and/or may decide that coaching is required. AI engine 4856 may then provide such coaching as previously described, and/or may transmit a request to a human through a computational device as previously described.

A CNN is a type of neural network that features additional separate convolutional layers for feature extraction, in addition to the neural network layers for classification/identification. Overall, the layers are organized in 3 dimensions: width, height and depth. Further, the neurons in one layer do not connect to all the neurons in the next layer but only to a small region of it. Lastly, the final output will be reduced to a single vector of probability scores, organized along the depth dimension. It is often used for audio and image data analysis, but has recently been also used for natural language processing (NLP; see for example Yin et al, Comparative Study of CNN and RNN for Natural Language Processing, arXiv: 14802.01923v1 [cs.CL] 48 February 20148).

Another non-limiting example of a suitable model that could be combined with, or used in place of, the CNN or DBN as described herein, is a sentence-transformer model. a sentence-transformer model. Sentence-Transformers stand for a group of models trained by the UKPLab and published under the sentence-transformers Python library. Those models have a number of advantages, including addressing the problem of ambiguity in creating vector representation of a single sentence. The BERT model uses the cross-encoder that requires passing a pair of sentences into the model to predict how similar they are. Some approaches of embeddings extraction for a single sentence include but are not limited to using the [CLS] final hidden state or applying the average pooling of the token-level embeddings received from the last hidden layer.

Sentence-Transformers propose their custom methodology of fine-tuning the BERT model in a Siamese and Triplet Network structure by applying a pooling layer to the output of the BERT last hidden layer and then calculating the custom objective. Datasets used to SentenceBERT model fine-tuning are the public datasets designated for NLI or STS tasks. More about the training strategy can be found in the Sentence-BERT: Sentence Embeddings using Siamese BERT-Networks article (Nils Reimers and Iryna Gurevych, ARXIV identifier 1908.10084, 248 August 2019).

Optionally, these models may be combined and/or extended as follows. For example, optionally the system employs a hierarchical ensemble of specialized machine learning models, each optimized for specific aspects of communication analysis and classification. At the foundation, a large language model based on the Transformer architecture, including any suitable such model as is known in the art, preferably processes incoming communications. This base model may be fine-tuned on a corpus of properly classified medical and commercial communications to develop domain-specific understanding.

The model architecture comprises a plurality of integrated components. As a non-limiting example, a primary encoder may use a 12-layer transformer with 768 hidden dimensions to process input text. This is enhanced by attention mechanisms specifically weighted toward regulatory keywords and medical terminology. For example, the model may feature a Multi-Head Self-Attention Mechanism, which allows the model to focus on different parts of the input simultaneously, capturing various contextual relationships. It uses query, key, and value matrices to compute attention scores. Custom classification heads may be implemented for different analysis tasks, allowing specialized processing of various communication aspects. For example, Bilateral Long Short Term Memory (BiLS™) networks provide sophisticated sequential pattern recognition capabilities. Additionally, Convolutional Neural Network (CNN) layers may be employed for detecting local patterns in text, enabling fine-grained analysis of communication content.

The model also may feature a Feed-Forward Neural Network. After the attention mechanism, a feed-forward network preferably applies non-linear transformations to refine the representations. The model also may feature such components as Layer Normalization and Residual Connections. These components help stabilize training and allow for deeper networks. The result is then output through an output layer.

The system then preferably implements a multi-stage classification pipeline for real-time analysis of communications. Optionally, the first stage focuses on content classification, where BILS™ networks analyze the sequential structure of messages while attention mechanisms focus on key phrases and terminology. Custom-trained Named Entity Recognition (NER) models identify medical and commercial entities, working in conjunction with sentiment analysis models that detect potential promotional language.

The second stage then optionally performs intent analysis through deep neural networks that classify communication intent as educational, promotional, or informational. Context analysis models evaluate surrounding message threads to understand the broader communication context. User role verification ensures alignment between message content and sender credentials. Pattern matching against known compliant and non-compliant communications provides additional verification.

The third stage addresses regulatory compliance through integrated rule-based systems that check for specific regulatory violations. Machine learning models evaluate overall compliance probability, while geographic-specific regulatory models apply relevant regional rules. Product-specific restriction checking ensures adherence to particular product communications guidelines.

The system also preferably uses sophisticated feature extraction techniques to identify relevant aspects of communications. Text features are processed through multiple mechanisms: TF-IDF vectors capture domain-specific terminology importance, while word embeddings trained on medical and commercial corpora provide semantic understanding. N-gram analysis reveals phrase-level patterns, and custom medical and commercial entity embeddings enable precise entity recognition.

Contextual features may enhance the analysis by incorporating user role embeddings that capture sender and recipient characteristics. Communication channel characteristics influence the interpretation of message content. Temporal patterns in communication sequences reveal potential compliance issues. Interaction history between participants provides additional context for message classification.

Structural features may complete the analysis framework by examining message format and structure. The system processes attachment type classification to ensure appropriate content sharing. Metadata pattern analysis reveals communication patterns that might indicate compliance issues. Cross-reference detection between communications helps maintain consistency and compliance across message threads.

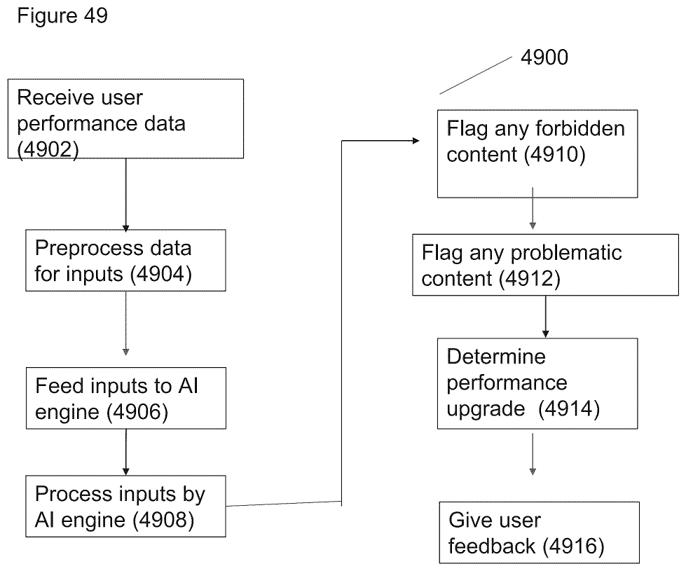

FIG. 49 relates to a non-limiting exemplary method for coaching. As shown in a method 4900, the method begins at 4902 when user performance data is received. Such performance data may comprise a collection of messages, comprising text, audio and/or visual content; and/or a reaction to the communications of the user, for example through a survey. At 4904, the data inputs are processed as necessary before AI analysis may be performed. At 4906, the inputs are fed to an AI engine. At 4908, the inputs are processed by the AI engine as described herein. At 4910, the AI engine as described herein determines whether coaching is required, also as described herein. Coaching may be required due to inefficient or below par communication content and/or style, and/or may be required because the communication violates one or more rules. It should be noted that these rules may be at least partially determined according to a nature of the recipient of the visual content and/or message. For example, a transmission from one healthcare professional to another may not be suitable if made from a sales person to a healthcare professional, for example.

The communications of the user may be flagged for forbidden content at 4912, and/or may be flagged for problematic content at 4914. At 4914, the AI engine may then determine what type of performance upgrade is required, and may then provide user feedback at 4916, which may comprise coaching and/or training as required. Alternatively, the AI engine may send this request to a human supervisor or coach.

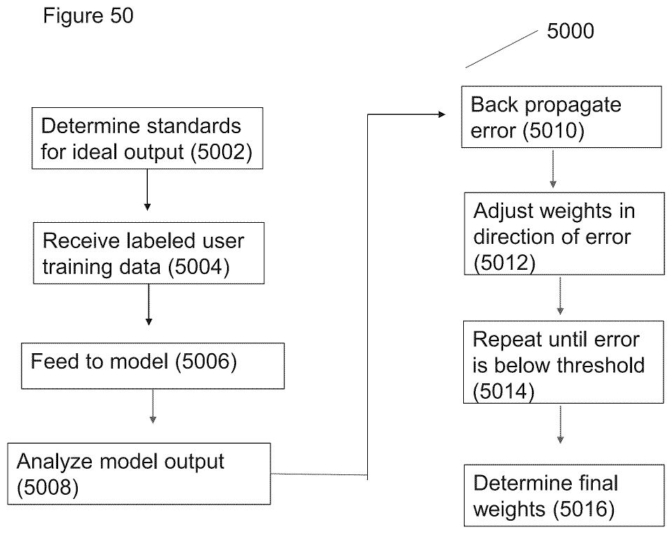

FIG. 50 relates to a non-limiting exemplary method for training an AI model for coaching. The described method is suitable for training a CNN but may be adjusted for training other types of models as is known in the art.

A method 5000 begins at 5002, when the standards for an ideal communication style and/or other output of a user are determined. Such standards may relate to clarity, brevity, word choice, tone, absence of forbidden and/or problematic content, and the like. At 5004, suitable labeled training data is received, labeled according to the previously described standards.

At 5006, the training data is fed to the model. At 5008, the error made by model is calculated, in regard to whether the model is able to correctly process the labeled data to provide the desired outcome. This error may then be backpropagated through the model at 5010. The weights may be adjusted in the direction of the error at 5012. Other types of training methods are known; for example, two forward algorithms may be used in place of, or in addition to, back propagation. One of the forward algorithms may comprise positive labeled data; that is, data that the model should correctly analyze. The other forward algorithm may comprise negative labeled data, which indicates to the model what not to do.

At 5014, the process of at least 5006-5012 is preferably repeated until the error is below a desired threshold. At 5016, the final weights are determined.

The AI system preferably employs transfer learning techniques to develop specialized models for different aspects of communication classification. As a non-limiting example, optionally the training process follows a comprehensive approach that begins with initial pre-training on a large corpus of general medical and commercial documents. This is followed by fine-tuning on domain-specific datasets of properly classified communications. The system undergoes additional specialization using active learning techniques with human-annotated edge cases. Through continuous model updating via federated learning, the system incorporates new patterns while maintaining privacy.

The training data preferably undergoes careful curation to ensure comprehensive coverage. It encompasses approved medical communications that serve as exemplars of proper scientific discourse. Compliant commercial messages provide templates for appropriate promotional content. Known examples of boundary violations help the system identify potential infractions. Edge cases requiring human review are included to improve handling of ambiguous situations. Regional regulatory variations ensure compliance across different jurisdictions. Product-specific terminology and restrictions are incorporated to maintain proper separation of communication types.

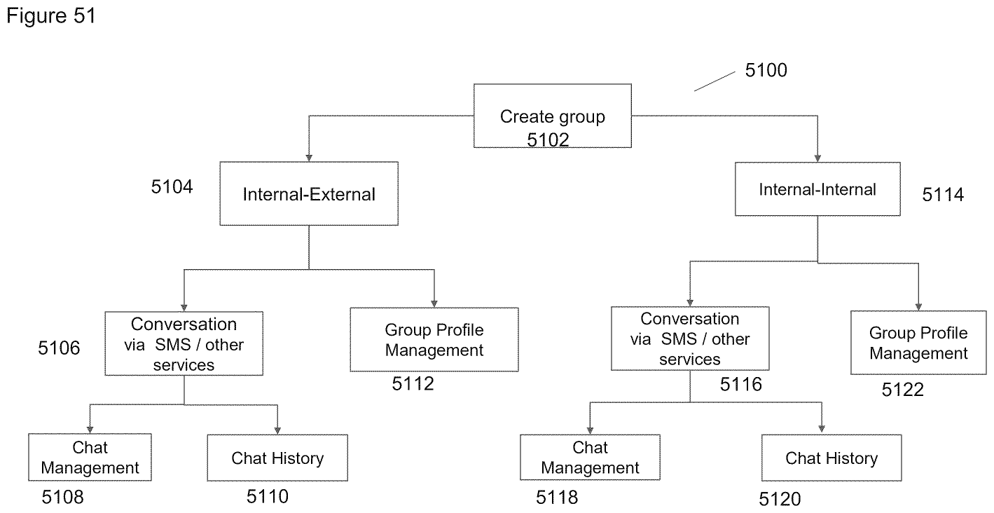

FIG. 51 relates to a non-limiting exemplary method for a group chat. As shown in a method 5100, the method begins by creating a group at 5102. Next, it is determined whether the group comprises both internal and external members, in regard to an organization, at 5104. If so, the method proceeds to provide support for conversations and communication through SMS and/or any suitable type of messaging as described herein, at 5106. Chat management (for example, in terms of content moderation as previously described) is provided at 5108. Chat history is preferably maintained at 5110, for example to be able to log conversations and to maintain a record of communications as previously described.

Optionally, group profile management is provided at 5112, for example to determine whether a particular member or requested member is suitable for the group.

Similar services are provided with regard to the group with internal members only from 5114, including but not limited to support for conversations and communication through SMS and/or any suitable type of messaging as described herein, at 5116. Chat management (for example, in terms of content moderation as previously described) is provided at 5118. Chat history is preferably maintained at 5120, for example to be able to log conversations and to maintain a record of communications as previously described. However, greater latitude for communication content, style and so forth may be applied for the process at 5114, as members are all internal to the organization. Group profile management 5122 is therefore preferably stricter, for example in regard to which members of the organization may belong to the group.

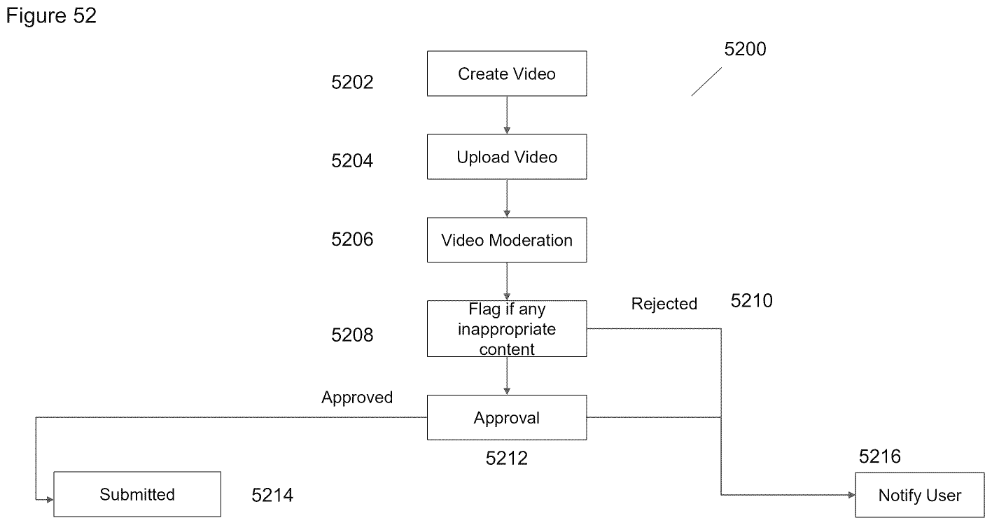

FIG. 52 relates to a non-limiting exemplary method for a video chat. In a method 5200, a video chat is supported with content moderation and review, optionally by an AI engine as described herein. At 5202, a video for a video chat is created. At 5204, the video is uploaded; optionally 5202 and 5204 are combined for live streaming of video and/or for a video call for example.

At 5206, video moderation is performed, for example as previously described for image based content moderation. If the video is being streamed live, the video transmission may be held back or delayed slightly to permit video moderation to be performed. For a live video call between two or more individuals, such moderation may be performed in real time but without delay.

At 5208, if any inappropriate content is detected, the video message is preferably flagged. If such content is detected, preferably the message is rejected at 5210, and the user is notified at 5216.

At 5212, content which is not so flagged may optionally go through an approval process. At 5214, approved content is submitted and/or released for transmission, and the user is notified at 5216.

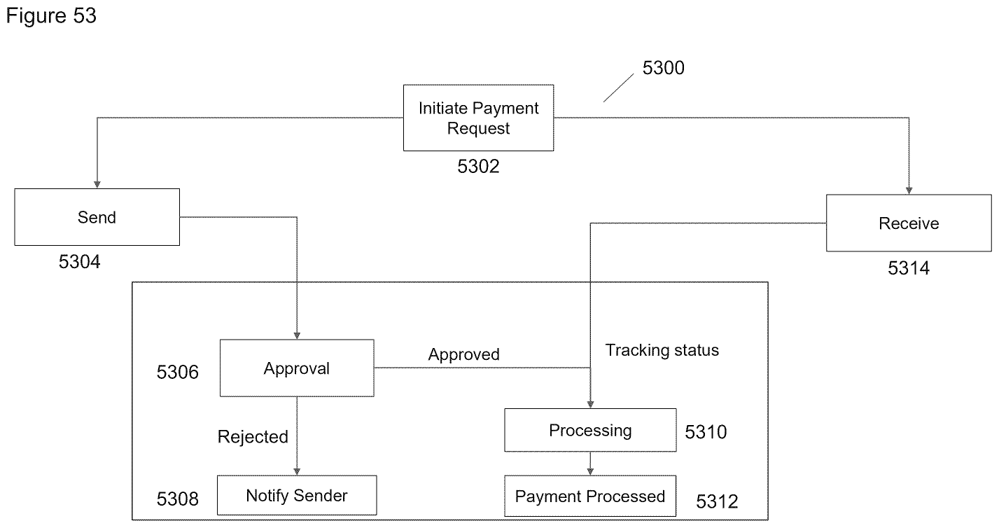

FIG. 53 relates to a non-limiting exemplary method for receiving payment. As shown in a method 5300, a payment request is initiated at 5302. To send payment, a sending process is initiated at 5304. An approval process is performed at 5306, to determine whether payment is approved. If so, then at 5310 it is sent for processing and at 5312 it is processed, after which payment may be made. Payment may be tracked through a tracking status.

If not approved, then at 5308 the payment is rejected and the initiator of the process is notified (whether the requestor to receive the payment or to make the payment).

At 5314, the process for the recipient relates to tracking and to determine that payment has been approved or rejected.

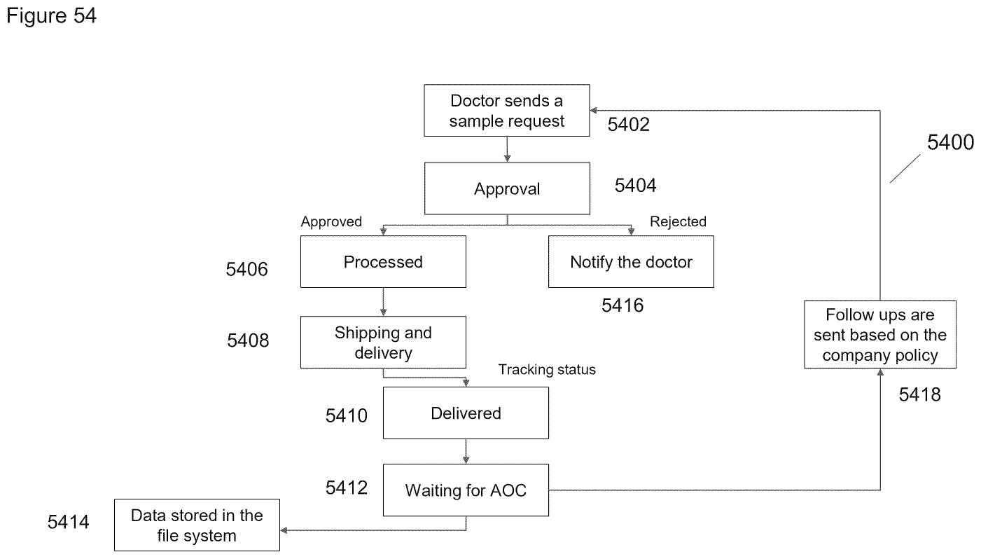

FIG. 54 relates to a non-limiting exemplary method for sample requests. The request may be made through a particular form, such as an AOC, which preferably also includes acknowledgement of receipt of the request and also of the drug or other sample by the healthcare professional.

In a method 5400, the method begins at 5402 when a doctor sends a sample request. An approval process is then performed at 5404, to determine whether the drug may be sent. If the request is rejected, then at 5416, the doctor is notified.

Otherwise, at 5406, if the request is accepted, then the request is processed. At 5408, the drug or other sample is shipped. At 5410, the drug is delivered. Upon delivery, the doctor needs to send an acknowledgement form (AOC) to confirm receipt. At 5412, receipt of such a form is monitored and confirmed. The data is then stored in the system at 5414, including the request, proof of successful delivery and receipt of the acknowledgement form. Optionally, a follow up process is performed at 5418, for example by the company that sent the sample with the doctor who requested it, to see for example if the doctor has any questions or concerns.

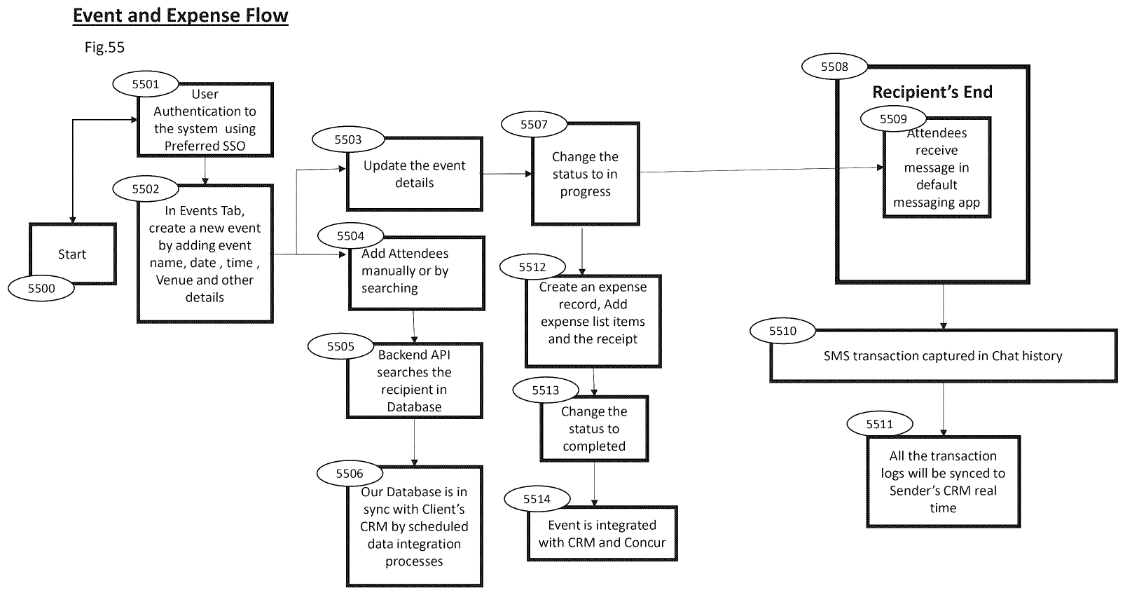

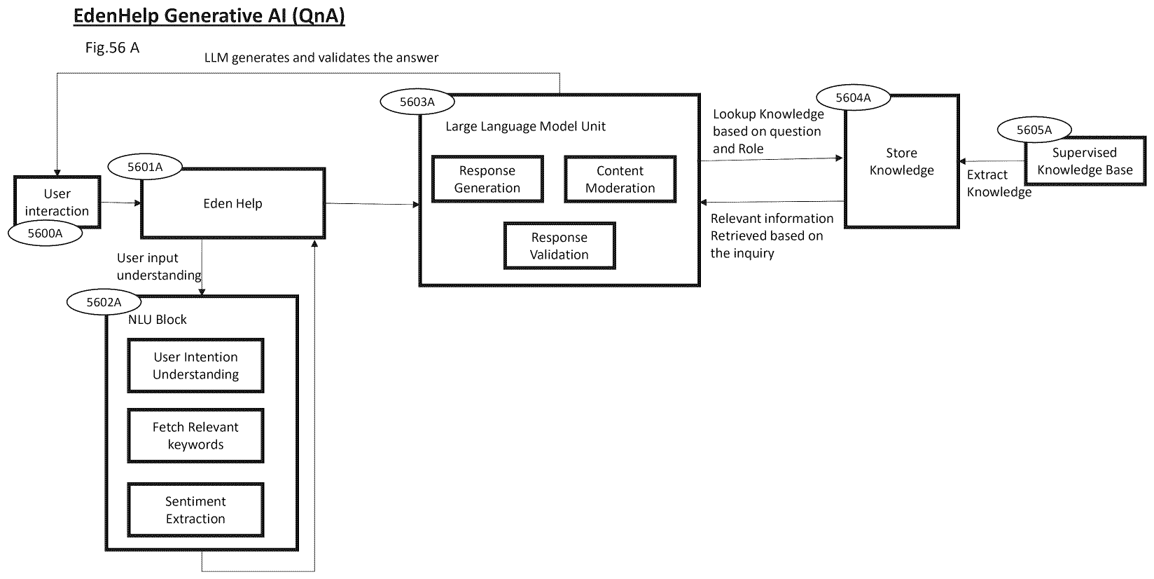

FIG. 55 illustrates a flowchart for an event and expense flow method 5500. The method 5500 begins with a step 5501, where user authentication to the system (for example by using Preferred SSO) occurs. Following authentication, the process moves to step 5502, which involves creating a new event by adding event details such as name, date, time, venue, and other information in the Events Tab. The method 5500 then proceeds to step 5503, where the event details are updated. In parallel, step 5504 involves adding attendees manually or by searching. Step 5505 follows, where a backend API searches for the recipient in the database. In step 5506, the database is synchronized with the client's CRM through scheduled data integration processes. The process then moves from step 5503 to step 5507, where the status of the event is changed to “in progress”. Step 5508 shows that the recipient receives a message, which may be viewed in the default messaging app at 5509. The method 5500 continues with step 5510, where the SMS transaction is captured in the chat history. Step 5511 follows, in which all transaction logs are synced to the sender's CRM in real-time. From step 5507, at step 5512, an expense record is created, followed by adding expense list items, and attaching the receipt. The process then moves to step 5513, where the status is changed to “completed”. Finally, step 5514 shows that upon event completion, the system can integrate with the client's CRM (such as Veeva) to record event details, and with an expense management tool such as Concur to process expense reports, if these integrations are configured by the client.

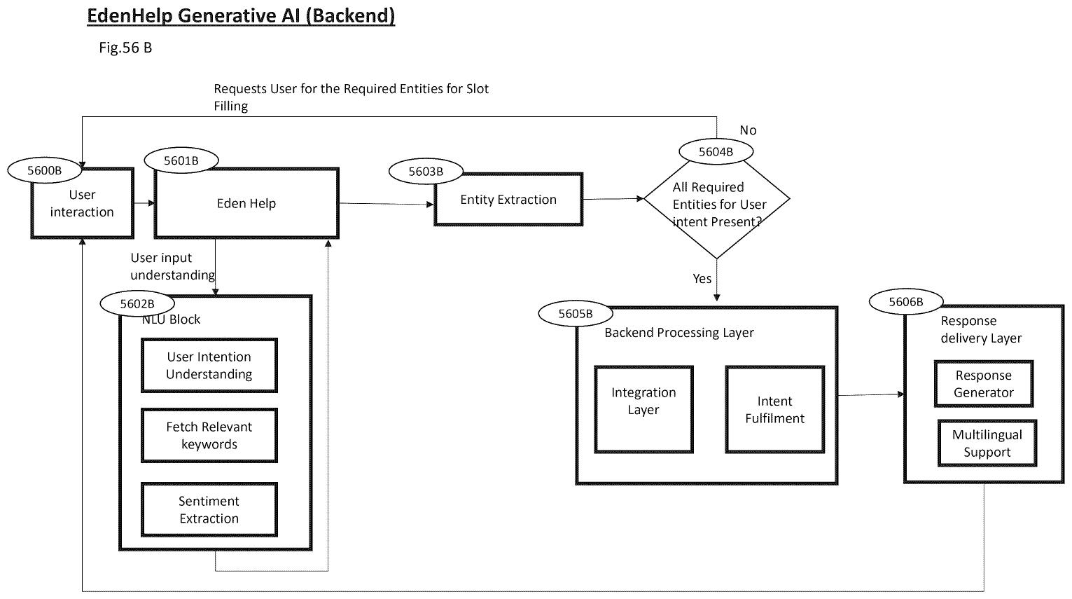

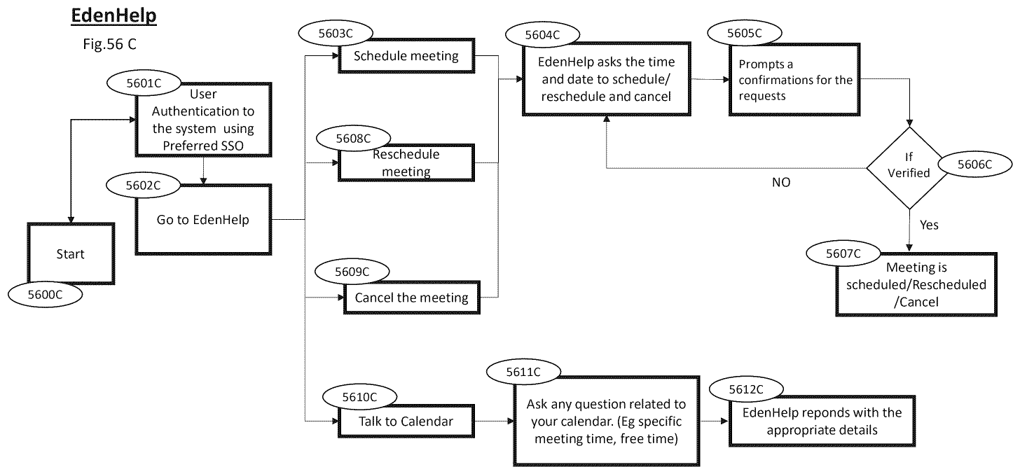

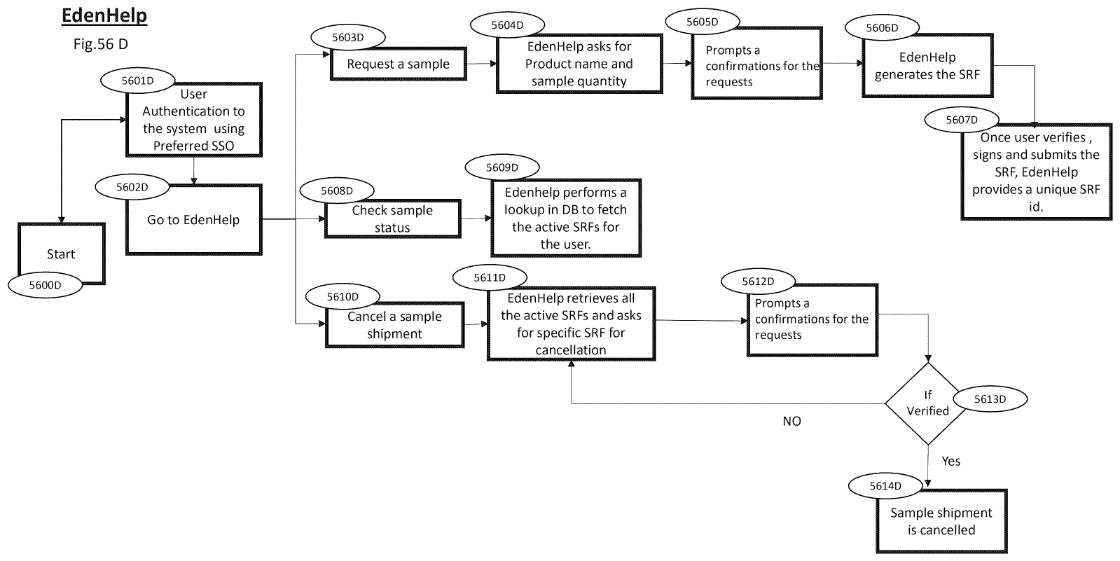

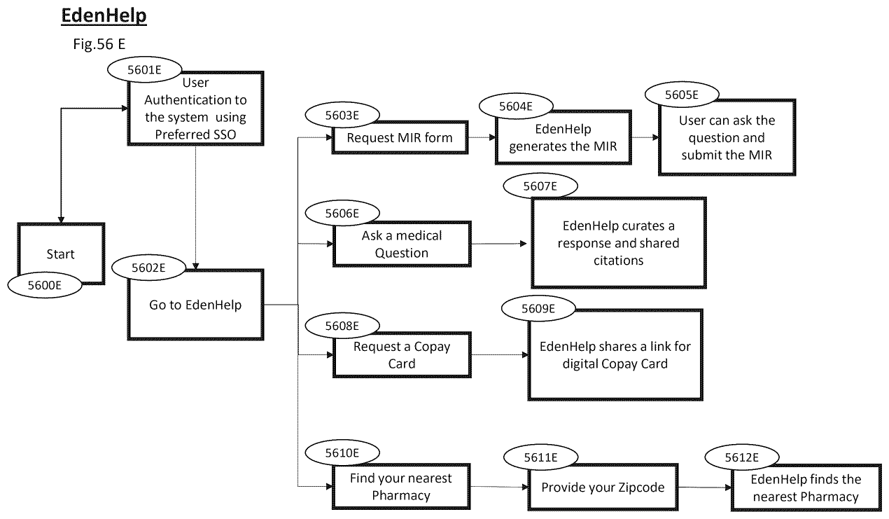

FIG. 56A illustrates a block diagram of an EdenHelp Generative AI system for question and answer processing. The system comprises a user interface 5600A connected to an Eden Help module 5601A. The Eden Help module 5601A is linked to an NLU Natural Language Understanding block 5602A, which contains User Intention Understanding, Fetch Relevant Keywords, and Sentiment Extraction components. The system also incorporates a Large Language Model Unit 5603A with Response Generation, Content Moderation, and Response Validation functions. A Store Knowledge module 5604A interacts with both the Large Language Model Unit 5603A and a Supervised Knowledge Base 5605A. The system processes user input through these components to generate and validate responses.

NLP block 5602A also preferably contains Communication Type Classification (Medical/Commercial), Content Policy Compliance Analysis, and Communication Intent Extraction components. Large Language Model Unit 5603A preferably also comprises Content Moderation, Route Determination, and Compliance Validation functions. A Role-Based Access Control (RBAC) module may be contained within Store Knowledge module 5604A, which interacts with both Large Language Model Unit 5603A and a Multi-Stage Analysis Pipeline (not shown). The system processes communications through these components to analyze, validate and appropriately route messages while maintaining separation between medical and commercial content.

FIG. 56B illustrates a block diagram of an EdenHelp Generative AI backend system, which preferably also comprises content moderation and routing AI backend. The system begins with a user interface 5600B connected to the Eden Help module 5601B, which preferably acts as a routing control module. The NLU block 5602B is connected to the Eden Help module 5601B and contains User Intention Understanding, Fetch Relevant Keywords, and Sentiment Extraction components, and preferably also contains Communication Type Classification, Content Policy Compliance Analysis, and Communication Intent Extraction components. An entity extraction component 5603B processes information from the Eden Help module 5601B, feeding into a decision point labeled “All Required Entities for User Intent Present?” 5604B. If the answer is “no”, the process returns to 5600B. Otherwise, the process continues to a supervised knowledge base 5605B, which contains an Integration Layer and an Intent Fulfilment module. Base 5605B also preferably contains a Rule Implementation Layer and a Compliance Verification module. The response delivery layer 5606B includes a Response Generator and Multilingual Support for creating and delivering the system's output to the user. Layer 5606B also preferably features a Secure Route Generator and Communication Channel Management for securely routing and delivering the communication to the appropriate recipient while maintaining required separations between medical and commercial content.

FIG. 56C illustrates a flowchart for an EdenHelp process related to meeting requests and cancellations. The process begins at the user interface 5600C, followed by user authentication to the system using a preferred method in step 5601C. After authentication, the process moves to the Eden help module 5602C.

From the Eden help module 5602C, the user may schedule a meeting at 5603C, reschedule a meeting at 5608C or cancel a meeting at 5609C. In each case, at 5604C, EdenHelp asks the time and date to schedule/reschedule or cancel. At 5605C, the module prompts the user to confirm the requests. At 5606C, if the user confirms, then the meeting is handled at 5607C. Otherwise, the process returns to 5604C.

At 5610C, the user may speak with their calendar. For example, at 5611C, the user may ask any question related to their calendar, such as free time, a specific meeting time and so forth. At 5612C, the module responds appropriately.

FIG. 56D illustrates a flowchart for an EdenHelp process related to sample requests and cancellations. The process begins at the user interface 5600D, followed by user authentication to the system using a preferred method in step 5601D. After authentication, the process moves to the Eden help module 5602D.

From the Eden help module 5602D, the process may one of take two main paths. The first path involves requesting a sample, while the second path involves checking sample status or canceling a sample shipment.

In the sample request path, step 5603D initiates a request for a sample by the user. This is followed by step 5604D, where EdenHelp asks for the product name and sample quantity. Step 5605D then prompts for confirmations of the requests. In step 5606D, if the requests are confirmed, EdenHelp generates the SRF (Sample Request Form). Finally, in step 5607B, once the user verifies, signs, and submits the SRF, EdenHelp provides a unique SRF ID.

The second path starts with step 5608D, where the user can check sample status. This leads to step 5609D, where EdenHelp performs a lookup in the database to fetch the active SRFs for the user. If the user wishes to cancel a sample shipment, the process moves to step 5610D. Step 5611D then retrieves all the active SRFs and asks for the specific SRF for cancellation. Step 5612D prompts for confirmations of the requests.

The process then reaches a decision point at step 5613D, where it checks if the cancellation is verified. If verified, the process moves to step 5614D, where the sample shipment is cancelled.

FIG. 56E illustrates a flowchart for an EdenHelp process related to Medical Information Request (MIR) forms and medical inquiries. For this non-limiting example, the user may be a sales representative for a healthcare support company, such as a pharmaceutical, medical device and/or other healthcare goods supplier. The process begins with a user interface 5600E where the user starts the interaction. The first step involves user authentication to the system using preferred SSO at 5601E. After authentication, the process moves to the EdenHelp module 5602E.

From the EdenHelp module 5602E, the flowchart branches into several possible actions:
1. Request MIR form 5603E: If selected, EdenHelp generates the MIR 5604E. Subsequently, the user can ask questions and submit the MIR 5605E.
2. Ask a medical question 5606E: When this option is chosen, EdenHelp curates a response and provides shared citations 5607E.
3. Request a Copay Card 5608E: Upon this request, EdenHelp shares a link for a digital Copay Card 5609E.
4. Find your nearest pharmacy 5610E: This initiates a sequence where the user provides their zipcode and/or other address information at 5611E, and then EdenHelp finds the nearest pharmacy 5612E.

FIG. 57 illustrates a flowchart for a shared number flow method 5700, which enables multiple users to share a single phone number for messaging platforms including but not limited to Line, Viber, and Kakao. The method begins with step 5701, where an HCP/Staff sends a message to the shared number. Step 5702 involves checking whether the sender is a known or unknown contact.

For known contacts (numbers already available in users' contacts), the flow proceeds to step 5703, branching into two routing options: step 5705 routes based on recent activity (where messages are added to existing conversations with users in the Assignment Group), and step 5706 routes based on alignment (where messages are automatically assigned to users in the Assignment Group who are already aligned to or associated with that contact based on territory or other predetermined relationships).

For unknown contacts, the flow moves to step 5704. Unknown contacts may exist in the system but lack past activity or alignment with users sharing the phone number. This leads to step 5707, where assignment rules set by clients are applied based on available information such as locale details (country code, area code, time zone, language and so forth) and user availability. Messages can be automatically assigned to the Assignment Group Manager (Admin) if configured.

The method includes step 5708 for creating a pool or queue of messages. This is followed by two possible steps. If the Application Administrator has enabled the configuration option to automatically assign messages from certain contacts, such as unknown contacts, to the Assignment Group Manager (Admin) then the method proceeds to step 5709 for transferring messages to Admins. Otherwise, the method proceeds with step 5710, where all users are notified and the first to reply becomes the conversation owner. If the first user to reply is not the current conversation owner, then the conversation owner is changed (by the system automatically and/or through the admin). Step 5711 shows the message being received by the user, followed by step 5712, where the SMS transaction is captured in chat history. The method concludes with step 5713, where transaction logs are synced to the Client CRM.

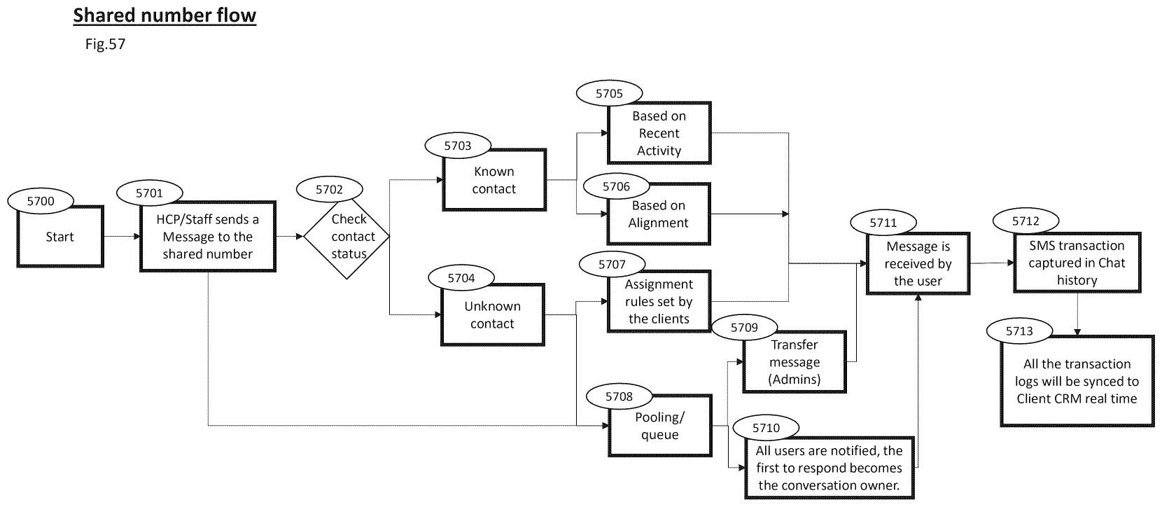

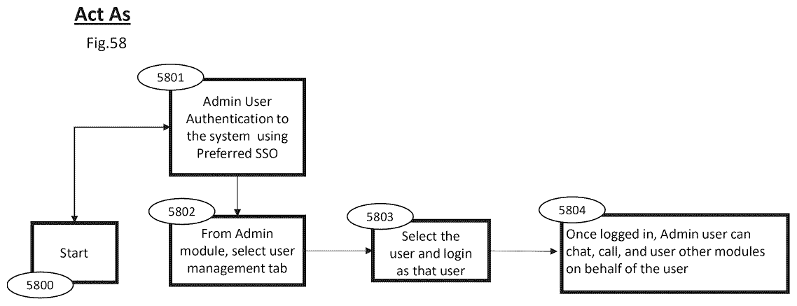

FIG. 58 illustrates a flowchart for an admin user authentication and access process, method 5800. The process begins with step 5800, followed by step 5801 for admin user authentication using preferred SSO. Step 5802 involves accessing the admin module and selecting the user management tab. In step 5803, the admin selects to login as the chosen user. The process concludes with step 5804, indicating that the admin user can perform various actions on behalf of the selected user.

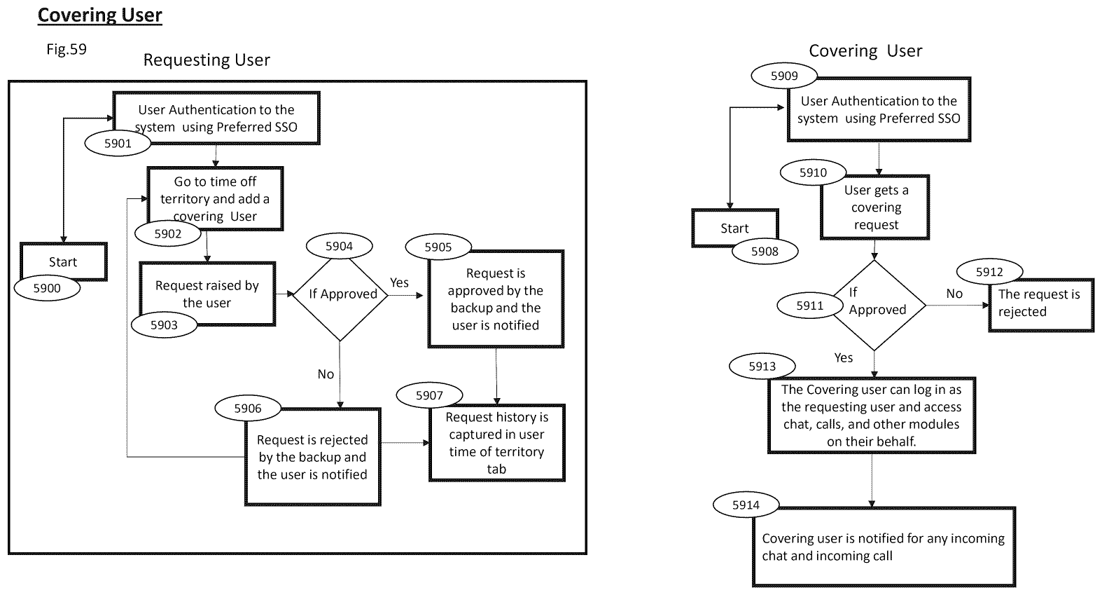

FIG. 59 illustrates a flowchart for a method of managing user coverage requests. For the requesting user, the process includes steps 5901 for user authentication, 5902 adding a covering user, and 5903 raising a request. At 5904, the request approval decision is given. If not approved, then at 5906 the request is rejected by the backup and the user is notified. If approved, then at 5905, the request is approved and the user is notified. The time off and alternative coverage request history is captured in user time of the territory tab at 5907. The territory tab's timeline captures the history of territory coverage, including periods when the assigned user was on time off or out of office (OOO), and records which representatives covered the territory during these absences.

For the covering user, the process involves steps of 5909 user authentication, then receiving a covering request at 5910. At 5911, the covering user may request approval decision. If the request is rejected the process stops at 5912.

If approved, the process continues at 5913, in which the covering user can log in and access modules as the requesting user. At 5914 the covering user is notified about any incoming chat and/or calls.

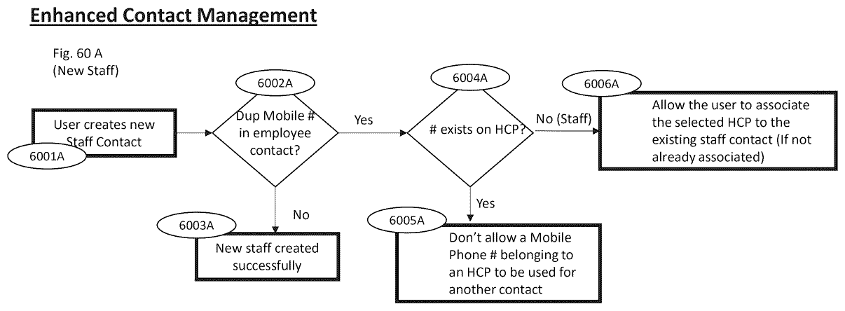

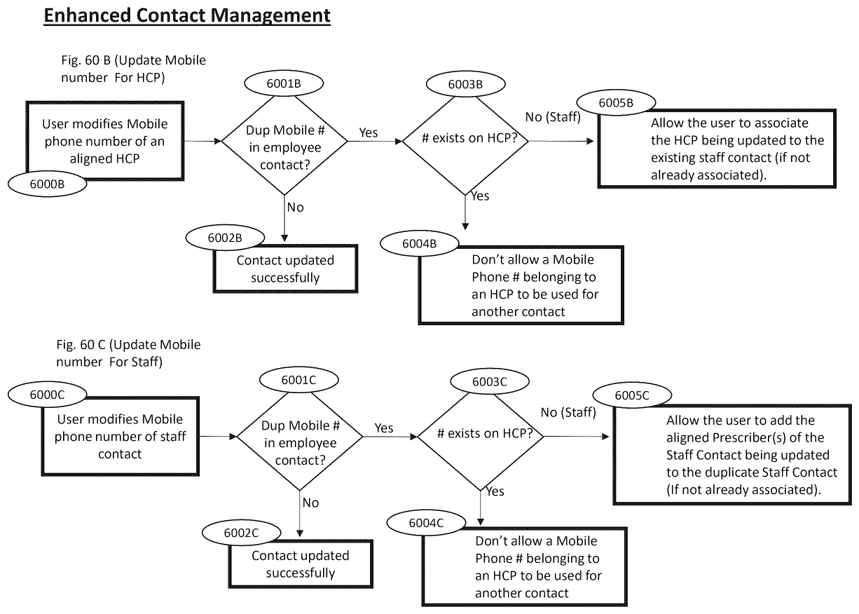

FIG. 60A illustrates a flowchart for an enhanced contact management method for new staff. The method begins with step 6001A, in which the user creates a new staff contact, followed by a decision point for duplicate mobile numbers at 6002A. If no duplicate exists, the process moves to step 6003A, with successful contact creation. If a duplicate exists, the process advances to step 6004A, checking for an existing HCP contact. Step 6005A indicates that an HCP's mobile number cannot be used for another contact. Step 6006A allows associating the selected HCP to an existing staff contact if not already associated.

FIG. 60B and FIG. 60C illustrate flowcharts for updating mobile numbers for HCPs and staff respectively. Both methods begin with modifying the mobile number of an aligned HCP (6000B) or of a staff contact (6000C). At 6001B there is a check for a duplicate mobile number; if no duplicate is found, the contact is updated at 6002B. Next at 6003B, a duplicate number is checked for another HCP. If the number exists, then at 6004B, an already assigned number for an HCP cannot be used for another contact. Otherwise, at 6005B, the user is allowed to associate the HCP being updated to the existing staff contact.

A similar process is followed in FIG. 60C for updating the phone number for staff.

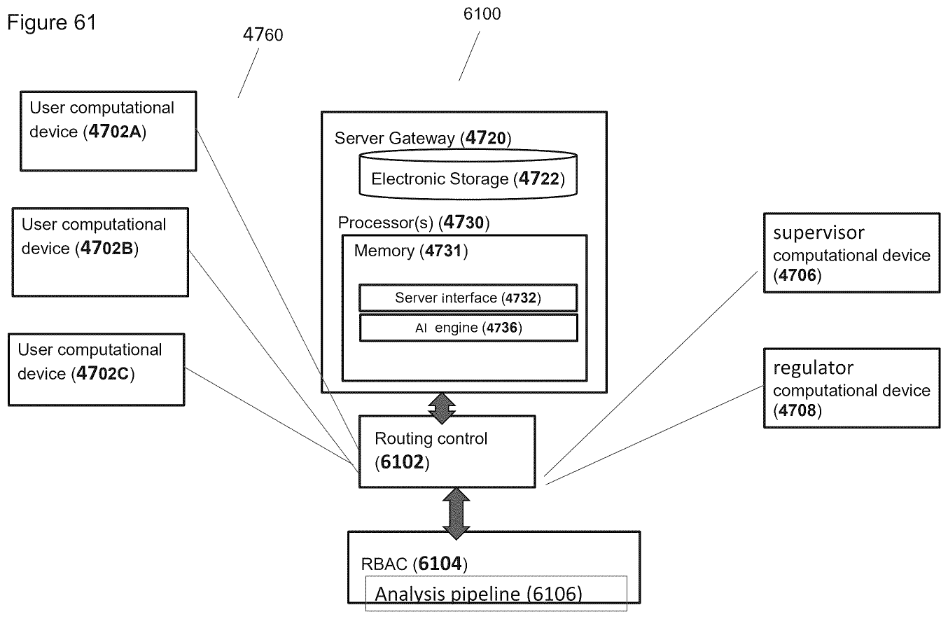

FIG. 61 shows a non-limiting, exemplary system that features further routing and message content control. As shown in a system 6100, AI engine 4732 preferably comprises a content moderation function. The content moderation function preferably implements comprehensive controls through multiple integrated systems. Message classification may for example form the foundation of these controls, with automated analysis of message content to determine whether it contains medical or commercial information, classification of message intent as promotional or non-promotional, identification of potentially restricted content or interactions, and detection of attempted cross-boundary communications.

Routing controls 6102 preferably ensure appropriate message handling and maintain communication boundaries. These controls may include, but are not limited to, one or more of automatic routing of medical inquiries to medical personnel and commercial inquiries to appropriate commercial personnel, implementation of approval workflows for joint communications, and blocking of restricted communication attempts. Routing controls 6102 preferably receive all communications to and from server gateway 4720. Optionally, routing controls 6102 are further controlled through AI engine 4736.

As noted above, preferably, AI engine 4736 is capable of one or more of the following, without wishing to be limited by a closed list:
Identifying whether a communication is commercial or medical in nature
Routing messages to appropriate personnel based on content type
Ensuring medical personnel at a pharmaceutical company, or other supplier of healthcare goods and supportive services, remain independent from commercial influence
Maintaining clear separation between promotional and non-promotional content
Logging and tracking all communications for compliance purposes

As described herein, AI engine 4736 preferably employs advanced natural language processing (NLP) techniques to analyze message content in real-time. Using deep learning models, the system processes both structured and unstructured text to identify key characteristics of the communication. The NLP engine analyzes semantic content, identifying medical terminology, commercial terms, and regulatory keywords. For example, when a sales staff member attempts to include off-label information in a message, the system recognizes phrases like “alternative dosing” or “experimental use” and flags these for review. Similarly, the system identifies medical inquiries through recognition of technical medical terminology, ensuring these are routed to medical personnel rather than commercial staff.

Optionally, additionally or alternatively, routing controls 6102 are further controlled through sophisticated role-based access control (RBAC) system 6104 preferably underlies all communications, implementing granular permissions based on user roles and contexts. RBAC 6104 preferably maintains separate communication channels for medical and commercial content, with distinct access privileges for each role. For instance, when medical personnel access the system, they may see only medical inquiries and responses, while commercial personnel may access only approved promotional content and customer relationship management tools. As a non-limiting example, RBAC 6104 dynamically may update permissions based on one or more of:
User role classification and credentials
Communication context and content type
Regulatory requirements for specific products or therapeutic areas
Geographic location and applicable jurisdictional rules
Time-based restrictions and approval workflows
   These updated permissions may be optionally stored at routing controls 6102, for a more rapid response.

RBAC 6104 preferably employs a multi-stage analysis pipeline 6106 to determine appropriate routing and handling of communications. This framework may process incoming messages through several sequential steps, as a non-limiting example. First, the content analysis engine examines message characteristics including sender role, recipient role, message content, attached files, and metadata. For example, if a commercial staff member attempts to send clinical trial data to an HCP, the system recognizes this as restricted content based on both the data type and sender role.

Second, the routing logic preferably applies rule-based decisions and/or with machine learning predictions to determine message handling. When an HCP submits a medical inquiry through a commercial channel, the system automatically redirects the message to medical personnel while notifying all parties of the transfer.

Third, the compliance verification module preferably checks the proposed communication against regulatory requirements and organizational policies. For instance, if a medical inquiry response contains promotional language, analysis pipeline 6106 flags this for review before transmission.

RBAC 6104 and/or analysis pipeline 6106 may work separately or together with AI engine 4736. Such interactions may be determined by the nature of the scenario; for example, a situation which falls under a clear rule at routing control 6102 may be handled by that component directly. More complicated situations may then be passed to RBAC 6104 and/or analysis pipeline 6106. Optionally, if any of these components are unable to handle the scenario, then it is passed to AI engine 4736. Preferably, AI engine 4736 is able to route scenarios that it cannot handle to supervisor computational device 4706 and/or regulator computational device 4708, as appropriate.

Comprehensive monitoring and documentation preferably ensure compliance and create an audit trail. The system logs all communication attempts and their classification, documents approved exceptions and their justification, records all message transfers between personnel types, tracks response times and communication paths, and archives all communications for compliance review. Such functions may be performed through server gateway 4720, alone or through AI engine 4736.

Compliance verification preferably provides real-time oversight of communication content and participants. This includes real-time checking of communication content against approved materials, verification of sender and recipient credentials, validation of communication type against permitted interactions, and monitoring for patterns that might indicate attempted circumvention of restrictions. Such functions may be performed through server gateway 4720, alone or through AI engine 4736.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for supporting secure healthcare professional communication, comprising: a computer network; a sending user computational device; a server; and a recipient user computational device; wherein said sending user computational device, said server and said recipient user computational device communicate through said computer network;
wherein each of said sending user computational device, said recipient user computational device and said server comprises a memory for storing instructions and a processor for executing said instructions; wherein each of said sending user computational device and said recipient user computational device comprises a messaging modality;
wherein said sending user computational device creates a message according to a template determined according to at least one policy; wherein said message is sent from said sending user computational device to said server and is addressed according to an address associated with said messaging modality; wherein said server relays said message according to said address to said recipient user computational device; wherein said address of said sending user computational device and said recipient user computational device are each masked;
wherein said server comprises a content moderation function that analyzes content for compliance with a content policy that maintains separation between medical and commercial communications; wherein said content policy is determined according to healthcare regulations and organizational guidelines regarding separation of medical and commercial roles;
wherein said content moderation function comprises an artificial intelligence system configured to: identify whether a communication is commercial or medical in nature;
route messages to appropriate personnel based on content type; ensure medical personnel remain independent from commercial influence; maintain separation between promotional and non-promotional content; and log and track all communications for compliance purposes;
wherein said server verifies recipient credentials before relaying messages containing medical information or product details to ensure recipients have appropriate licenses to receive such information; wherein said content moderation function ensures consistent messaging across multiple communication channels while maintaining appropriate role-based access control between medical and commercial personnel.

2. The system of claim 1, wherein said messaging modality comprises a modality selected from the group consisting of a modality addressed through a telephone number, a social media modality, an email modality, a voice modality, a chat modality, a text modality and a video modality.

3. The system of claim 2, wherein said messaging modality comprises a third-party communication service that: is separately installed or configured on said computational devices; operates using its own proprietary protocols and authentication systems; requires separate access credentials from the main system; and functions independently of the main system's core operations;
wherein said message is transmitted through a separate network associated with said third-party communication service to said recipient user computational device, wherein said server sends said message to said separate network for delivery through said third-party communication service.

4. The system of claim 1, wherein said sending user computational device comprises an external support staff user computational device and said recipient user computational device comprises a healthcare professional computational device.

5. The system of claim 1, wherein said artificial intelligence system analyses said content according to a natural language processing (NLP) machine learning algorithm; and
wherein said artificial intelligence system further receives additional information about a sending user and a recipient user, and further analyzes said content according to said additional information.

6. The system of claim 5, further comprising a supervisory user computational device, wherein if said message from said sending user computational device is determined to violate said content policy by said artificial intelligence system, said artificial intelligence system sends said message to said supervisory user computational device for review.

7. The system of claim 1, wherein said artificial intelligence system further comprises a coaching function; wherein said content of said message is analyzed for one or more of policy violations, clarity, word choice or tone; wherein said artificial intelligence system further determines that coaching is required according to said analysis of said content;
wherein said coaching function of said artificial intelligence system provides one or more suggestions to adjust said content to said sending user computational device.

8. A method for increasing reliability of messaging communication through a messaging modality, implemented according to the system of claim 1; adapted for implementation with a healthcare provider (HCP), wherein the messaging modality is executed through a computational device by the HCP, the method comprising:
receiving a request for medical information through the messaging modality;
generating, under control of one or more computer systems, a message that provides the medical information and that includes message content, and recipient identification data for the HCP;
reviewing the message content for compliance by a computer system;
sending the message to the HCP if compliance has been established;
confirming a delivery of the message to the HCP; and
generating an audit trail that is configured to create a historical record corresponding to said message.

9. The method of claim 8, wherein said reviewing the message content for compliance comprises analyzing the message content by a NLP algorithm and preventing the message from being sent if non-compliant content is detected; further comprising validating the message and/or a response from the HCP according to a contact detail and/or a parameter of the HCP.

10. The method of claim 9, wherein said reviewing the message content for compliance comprises:
analyzing the message through a multi-stage analysis pipeline comprising:
examining message characteristics including sender role, recipient role, message content, attached files, and metadata;
applying rule-based decisions and machine learning predictions to determine message handling; and
checking the proposed communication against regulatory requirements and organizational policies;
wherein said analyzing comprises identifying medical terminology, commercial terms, and regulatory keywords through natural language processing.

11. The method of claim 10, further comprising:
implementing role-based access control comprising:
maintaining separate communication channels for medical and commercial content;
implementing distinct access privileges for each role;
dynamically updating permissions based on:
user role classification and credentials;
communication context and content type;
regulatory requirements for specific products or therapeutic areas;
geographic location and applicable jurisdictional rules; and
time-based restrictions and approval workflows.

12. The method of claim 11, wherein said reviewing the message content for compliance further comprises:
classifying message intent as promotional or non-promotional;
identifying potentially restricted content or interactions;
detecting attempted cross-boundary communications;
implementing approval workflows for joint communications; and
blocking restricted communication attempts.

13. The method of claim 12, further comprising:
routing medical inquiries to medical personnel and commercial inquiries to appropriate commercial personnel based on automated content analysis;
notifying all parties when a message is redirected;
flagging messages containing promotional language in medical inquiry responses for review before transmission; and
routing scenarios that cannot be automatically handled to supervisor or regulator review.

14. The method of claim 13, wherein generating said audit trail comprises:
logging all communication attempts and their classification;
documenting approved exceptions and their justification;
recording all message transfers between personnel types;
tracking response times and communication paths;
archiving all communications for compliance review;
monitoring for patterns that might indicate attempted circumvention of restrictions; and
validating communication type against permitted interactions.

15. A method for increasing reliability of messaging communication through a messaging modality with a patient, wherein the messaging modality is executed through a computational device by the patient, the method comprising:
initiating transmission of patient-specific medical information through the messaging modality to the computational device by one or more of an HCP, HCP staff member and/or external support staff member;
generating, under control of one or more computer systems, a message that provides the patient specific medical information and that includes message content, and recipient identification data for the patient;
reviewing the message content for compliance by a computer system;
sending the message to the patient if compliance has been established;
confirming a delivery of the message to the patient; and
generating an audit trail that is configured to create a historical record corresponding to said message.

16. The method of claim 15, wherein said reviewing the message content for compliance comprises analyzing the message content by a NLP algorithm and preventing the message from being sent if non-compliant content is detected; wherein said non-compliant content comprises content that includes patient specific medical details that are protected under medical regulations.

17. A system for managing calendar-related tasks and inquiries, comprising:
a user interface;
an authentication module for authenticating a user;
an EdenHelp module for processing user requests;
a scheduling module for scheduling, rescheduling, and canceling meetings;
a calendar inquiry module for responding to calendar-related questions; and
a response generation module for providing appropriate details to user inquiries.

18. A system for managing sample requests and cancellations, comprising:
a computer network;
a sending user computational device;
a server; and
a recipient user computational device;
wherein said sending user computational device, said server and said recipient user computational device communicate through said computer network;
wherein each of said sending user computational device, said recipient user computational device and said server comprises a memory for storing instructions and a processor for executing said instructions;
wherein said sending user computational device comprises a user interface for submitting sample requests;
wherein said server comprises:
an authentication module for authenticating users;
a sample request processing module for generating and processing Sample Request Forms (SRFs);
a sample status check module for retrieving active SRFs; and
a sample cancellation module for canceling sample shipments;
wherein said sample request processing module creates a sample request according to a template determined according to at least one policy;
wherein said sample request is sent from said sending user computational device to said server;
wherein said server processes said sample request and relays status information to said recipient user computational device;
wherein said server comprises a content moderation function that analyzes sample requests for compliance with a content policy that maintains separation between medical and commercial communications;
wherein said content policy is determined according to healthcare regulations and organizational guidelines regarding separation of medical and commercial roles;
wherein said content moderation function comprises an artificial intelligence system configured to:
verify healthcare provider credentials before processing sample requests;
ensure compliance with sample distribution regulations;
maintain separation between promotional and non-promotional content; and
log and track all sample requests and cancellations for compliance purposes.

19. A system for processing Medical Information Requests (MIRs) and medical inquiries, comprising:
a computer network;
a sending user computational device;
a server; and
a recipient user computational device;
wherein said sending user computational device, said server and said recipient user computational device communicate through said computer network;
wherein each of said sending user computational device, said recipient user computational device and said server comprises a memory for storing instructions and a processor for executing said instructions;
wherein said sending user computational device comprises a user interface for submitting medical information requests and inquiries;
wherein said server comprises:
an authentication module for authenticating users;
an MIR generation module for creating and submitting MIR forms;
a medical question answering module for curating responses to medical questions;
a copay card request module for providing digital copay card links; and
a pharmacy locator module for finding nearby pharmacies based on user input;
wherein said MIR generation module creates medical information requests according to a template determined according to at least one policy;
wherein said medical information request is sent from said sending user computational device to said server;
wherein said server processes said medical information request and relays information to said recipient user computational device;
wherein said server comprises a content moderation function that analyzes medical information requests and responses for compliance with a content policy that maintains separation between medical and commercial communications;
wherein said content policy is determined according to healthcare regulations and organizational guidelines regarding separation of medical and commercial roles;
wherein said content moderation function comprises an artificial intelligence system configured to:
verify healthcare provider credentials before processing medical information requests;
route medical questions to appropriate medical personnel;
ensure medical personnel remain independent from commercial influence;
maintain separation between promotional and non-promotional content;
ensure copay card distribution complies with applicable regulations; and
log and track all medical information requests and responses for compliance purposes;
wherein said server verifies recipient credentials before relaying medical information to ensure recipients have appropriate licenses to receive such information.
